(19) United States
(12) Patent Application Publication
(10) Pub. No.: US 2007/0128694 A1
(43) Pub. Date: Jun. 7, 2007

(54) Compositions and methods relating to the daptomycin biosynthetic gene cluster

(75) Inventors: Vivian Pak Woon Miao, Surrey (CA); Paul Brian, Waltham, MA (US); Richard H. Baltz, Lincoln, MA (US); Marie Francoise Coeffet-LeGal, Arlington, MA (US)

Correspondence Address: CUBIST PHARMACEUTICALS, INC., 65 HAYDEN AVENUE, LEXINGTON, MA, 02421, US

(73) Assignee: Cubist Pharmaceuticals, Inc.

(21) Appl. No.: 11/510,972

(22) Filed: Aug. 28, 2006

Related U.S. Application Data
(62) Division of application No. 10/211,028, filed on Jul. 31, 2002, now abandoned.
(63) Continuation-in-part of application No. PCT/US01/32354, filed on Oct. 17, 2001.
(60) Provisional application No. 60/379,866, filed on May 10, 2002.
(60) Provisional application No. 60/310,385, filed on Aug. 6, 2001.

(51) Int. Cl. C12P 21/06; C12N 9/10; C07H 21/04; C12N 15/74; C12N 1/21

(52) U.S. Cl. 435/69.1; 536/23.2; 435/193; 435/252.3; 435/471

(57) ABSTRACT

The invention provides nucleic acid molecules comprising all or a part of a daptomycin biosynthetic gene cluster. The daptomycin biosynthetic gene cluster may be derived from Streptomyces, preferably from S. roseosporus. The invention also provides other nucleic acid molecules from S. roseosporus. The invention further provides polypeptides encoded by the nucleic acid molecules, antibodies that specifically bind to the polypeptides, and methods of using the nucleic acid molecules, polypeptides and antibodies to produce daptomycin and other compounds.

### CROSS REFERENCE TO RELATED APPLICATIONS

This application is a divisional of U.S. patent application Ser. No. 10/211,028 filed Jul. 31, 2002, which claims priority to U.S. Provisional Application 60/310,385 filed Aug. 6, 2001, which is also a continuation-in-part of PCT/US01/32354, which claims priority to U.S. Provisional Application No. 60/379,866 filed May 10, 2002.

### BACKGROUND OF THE INVENTION

Bacteria, including actinomycetes, and fungi synthesize a diverse array of low molecular weight peptide and polyketide compounds (approx. 2-48 residues in length). The biosynthesis of these compounds is catalyzed by non-ribosomal peptide synthetases (NRPSs) and by polyketide syntheses (PKSs). The NRPS process, which does not involve ribosome-mediated RNA translation according to the genetic code, is capable of producing peptides that exhibit enormous structural diversity, compared to peptides translated from RNA templates by ribosomes. These include the incorporation of D- and L-amino acids and hydroxy acids; variations within the peptide backbone which form linear, cyclic or branched cyclic structures; and additional structural modifications, including oxidation, acylation, glycosylation, N-methylation and heterocyclic ring formation. Many non-ribosomally synthesized peptides have been found which have useful pharmacological (e.g., antibiotic, antiviral, antifungal, antiparasitic, siderophore, cytostatic, immunosuppressive, anti-cholesterolemic and anticancer), agrochemical or physicochemical (e.g., biosurfactant) properties.

Non-ribosomally synthesized peptides are assembled by large (e.g., about 200-2000 kDa), multifunctional NRPS enzyme complexes comprising one or more subunits. Examples include daptomycin, vancomycin, echinocandin and cyclosporin. Likewise, polyketides are assembled by large multifunctional PKS enzyme complexes comprising one or more subunits. Examples include erythromycin, tylosin, monensin and avermectin. In some cases, complex molecules can be synthesized by mixed PKS/NRPS systems. Examples include rapamycin, bleomycin and epothilone.

An NRPS usually consists of one or more open reading frames that make up an NRPS complex. The NRPS complex acts as a protein template, comprising a series of protein biosynthetic units configured to bind and activate specific building block substrates and to catalyze peptide chain formation and elongation. (See, e.g., Konz and Marahiel, Chem. Biol., 6, pp. 39-48 (1999) and references cited therein; von Döhren et al., Chem. Biol., 6, pp. 273-279, (1999) and references cited therein; and Cane and Walsh, Chem. Biol., 6, pp. 319-325, (1999), and references cited therein—each hereby incorporated by reference in its entirety). Each NRPS or NRPS subunit comprises one or modules. A “module” is defined as the catalytic unit that incorporates a single building block (e.g., an amino acid) into the growing peptide chain. The order and specificity of the biosynthetic modules that form the NRPS protein template dictates the sequence and structure of the ultimate peptide products.

Each module of an NRPS acts as a semi-autonomous active site containing discrete, folded protein domains responsible for catalyzing specific reactions required for peptide chain elongation. A minimal module (in a single module complex) consists of at least two core domains: 1) an adenylation domain responsible for activating an amino acid (or, occasionally, a hydroxy acid); and 2) a thiolation or acyl carrier domain responsible for transferring activated intermediates to an enzyme-bound pantetheine cofactor. Most modules also contain 3) a condensation domain responsible for catalyzing peptide bond formation between activated intermediates. See FIG. 9. Supplementing these three core domains are a variable number of additional domains which can mediate, e.g., N-methylation (M or methylation domain) and L- to D-conversion (E or epimerization domain) of a bound amino acid intermediate, and heterocyclic ring formation (Cy or cyclization domain). The domains are usually characterized by specific amino acid motifs or features. It is the combination of such auxiliary domains acting locally on tethered intermediates within nearby modules that contributes to the enormous structural and functional diversity of the mature peptide products assembled by NRPS and mixed NRPS/PKS enzyme complexes.

The adenylation domain of each minimal module catalyzes the specific recognition and activation of a cognate amino acid. In this early step of non-ribosomal peptide biosynthesis, the cognate amino acid of each NRPS module is bound to the adenylation domain and activated as an unstable acyl adenylate (with concomitant ATP-hydrolysis). See, e.g., Stachelhaus et al., Chem. Biol. 6: 493-505 (1999) and Challis et al., Chem. Biol. 7: 211-224 (2000), each incorporated herein by reference in its entirety. In most NRPS modules, the acyl adenylate intermediate is next transferred to the T (thiolation) domain (also referred to as a peptidyl carrier protein or PCP domain) of the module where it is converted to a thioester intermediate and tethered via a transthiolation reaction to a covalently bound enzyme cofactor (4′-phosphopantetheinyl (4′-PP) intermediate). Modules responsible for incorporating D-configured or N-methylated amino acids may have extra modifying domains which, in several NRPSs studied, are located between the A and T domains.

The enzyme-bound intermediates in each module are then assembled into the peptide product by stepwise condensation reactions involving transfer of the thioester-activated carboxyl group of one residue in one module to, e.g., the adjacent amino group of the next amino acid in the next module while the intermediates remain linked covalently to the NRPS. Each condensation reaction is catalyzed by a condensation (C) domain which is usually positioned between two minimal modules. The number of condensation domains in a NRPS generally corresponds to the number of peptide bonds present in the final (linear) peptide. An extra C domain has been found in several NRPSs (e.g., at the amino terminus of cyclosporin synthetase and the carboxyl terminus of rapamycin; see, e.g., Konz and Marahiel, supra) that has been proposed to be involved in peptide chain termination and cyclization reactions. Many other NRPS complexes, however, release the full length chain in a reaction catalyzed by a C-terminal thioesterase (Te) domain (of approximately 28K-35K relative molecular weight).

Thioesterase domains of most NRPS complexes use a catalytic triad (similar to that of the well-known chymotrypsin mechanism) which includes a conserved serine (less often a cysteine or aspartate) residue in a conserved three-dimensional configuration relative to a histidine and an acidic residue. See, e.g. V. De Crecy-Lagard in Comprehensive Natural Products Chemistry, Volume 4, ed. J. W. Kelly (New York: Elsevier), 1999, pp. 221-238, each incorporated herein by reference in its entirety. Thioester cleavage is a two step process. In the first (acylation) step, the full length peptide chain is transferred from the thiol tethered enzyme intermediate in the thiolation domain (see above) to the conserved serine residue in the Te domain, forming an acyl-O-Te ester intermediate. In the second (deacylation) step, the Te domain serine ester intermediate is either hydrolyzed (thereby releasing a linear, full length product) or undergoes cyclization, depending on whether the ester intermediate is attacked by water (hydrolysis) or by an activated intramolecular nucleophile (cyclization).

Sequence comparisons of C-terminal thioesterase domains from diverse members of the NRPS superfamily have revealed a conserved motif comprising the serine catalytic residue (GXSXG motif), often followed by an aspartic acid residue about 25 amino acids downstream from the conserved serine residue. A second type of thioesterase, a free thioesterase enzyme, is known to participate in the biosynthesis of some peptide and polyketide secondary metabolites. See e.g., Schneider and Marahiel, Arch. Microbiol., 169, pp. 404-410 (1998), and Butler et al., Chem. Biol., 6, pp. 87-292 (1999), each incorporated herein by reference in its entirety. These thioesterases are often required for efficient natural product synthesis. Butler et al. have postulated that the free thioesterase found in the polyketide tylosin gene cluster—which is required for efficient tylosin production—may be involved in editing and proofreading functions.

The modular organization of the NRPS multienzyme complex is mirrored at the level of the genomic DNA encoding the modules. The organization and DNA sequences of the genes encoding several different NRPSs have been studied. (See, e.g., Marahiel, Chem. Biol., 4, pp. 561-567 (1997), incorporated herein by reference in its entirety). Conserved sequences characterizing particular NRPS functional domains have been identified by comparing NRPS sequences derived from many diverse organisms and those conserved sequence motifs have been used to design probes useful for identifying and isolating new NRPS genes and modules.

The modular structures of PKS and NRPS enzyme complexes can be exploited to engineer novel enzymes having new specificities by changing the numbers and positions of the modules at the DNA level by genetic engineering and recombination in vivo. Functional hybrid NRPSs have been constructed, for example, based on whole-module fusions. See, e.g., Gokhale et al., Science, 284, pp. 482-485 (1999); Mootz et al., Proc. Natl. Acad. Sci. U.S.A., 97, pp. 5848-5853 (2000), incorporated herein by reference in their entirety. Recombinant techniques may be used to successfully swap domains originating from a heterologous PKS or NRPS complex. See, e.g., Schneider et al., Mol. Gen. Genet., 257, pp. 308-318 (1998); McDaniel et al., Proc. Natl. Acad. Sci. U.S.A., 96, pp. 1846-1851 (1999); U.S. Pat. Nos. 5,652,116 and 5,795,738; and International Publication WO 00/56896; incorporated herein by reference in their entirety.

Engineering a new substrate specificity within a module by altering residues which form the substrate binding pocket of the adenylation domain has also been described. See, e.g., Cane and Walsh, Chem. Biol., 6, 319-325 (1999); Stachelhaus et al., Chem. Biol., 6, 493-505 (1999); and WO 00/52152; each incorporated herein by reference in its entirety. By comparing the sequence of the B. subtilis peptide synthetase GrsA adenylation domain (PheA) (whose structure is known) with sequences of 160 other adenylation domains from pro- and eukaryotic NPRSs, for example, Stachelhaus et al. (supra) and Challis et al., Chem. Biol., 7, pp. 211-224 (2000) defined adenylation (A) domain signature sequences (analogous to codons of the genetic code) for a variety of amino acid substrates. From the collection of those signature sequences, a putative NRPS selectivity-conferring code (with degeneracies like the genetic code) was formulated.

The ability to engineer NRPSs having new modular template structures and new substrate specificities by adding, deleting or exchanging modules (or by adding, deleting or exchanging domains within one or more modules) will enable the production of novel peptides having altered and potentially advantageous properties. A combinatorial library comprising over 50 novel polyketides, for example, was prepared by systematically modifying the PKS that synthesizes an erythromycin precursor (DEBS) by substituting counterpart sequences from the rapamycin PKS (which encodes alternative substrate specificities). See, e.g., WO 00/63361 and McDaniel et al., (1999), supra, each incorporated herein by reference in its entirety.

A number of bacteria that produce antibiotics and other potentially toxic compounds synthesize ATP-binding cassette (ABC) transporters. ABC transporters use proton-dependent transmembrane electrochemical potential to export toxic cellular metabolites such as antibiotics, and to import materials from the environment, e.g. iron or other metals. There are three types of ABC transporters and genes encoding pumps responsible for antibiotic resistance, and they are often linked to the biosynthetic cluster in antibiotic producer organisms (e.g. actinorhodin resistance in Streptomyces coelicolor). See, e.g., Mendez et al., FEMS Microbiol. Lett. 158: 1-8 (1998), herein incorporated by reference. All have ATP-binding regions that include Walker A and B motifs. Id. Type I systems involve separate genes for a hydrophilic ATP-binding domain and a hydrophobic integral membrane domain. Type III systems involve a single gene encoding a protein with a hydrophobic N-terminus and a hydrophilic, ATP-binding C-terminus. Type II transporters have no hydrophobic domain, and two sets of Walker motifs, in the order A:B:A:B.

The Streptomyces glaucescens genes, StrV (PIR Accession No. S57561) and StrW (PIR Accession No. S57562) encode type III transporters associated with resistance to streptomycin-related compounds. Both genes are within a 5′-hydroxystreptomycin antibiotic biosynthetic gene cluster. See, e.g., Beyer et al., Mol. Gen. Genet. 250: 775-84 (1996), herein incorporated by reference. Resistance to doxorubicin and related antibiotics is conferred by two type I transporters in Streptomyces peucetius, which are encoded by drrA and drrB. See, e.g., Guifoile et al., Proc. Natl. Acad. Sci. USA 88:8553-57 (1991), herein incorporated by reference. Further, homologs of drrAB isolated from Streptomyces rochei confer multidrug resistance when expressed under control of the actinorhodin PKS promoter in S. lividans. See, e.g., Fernandez-Moreno et al., J. Bacteriol. 179: 6929-36 (1998), herein incorporated by reference.

Daptomycin (described by R. H. Baltz in Biotechnology of Antibiotics, 2nd Ed., ed. W. R. Strohl (New York: Marcel Dekker, Inc.), 1997, pp. 415-435) is an example of a non-ribosomally synthesized peptide made by a NRPS. Daptomycin, also known as LY146032, is a cyclic lipopeptide antibiotic that is produced by the fermentation of Streptomyces roseosporus. Daptomycin is a member of the factor A-21978C type antibiotics of S. roseosporus and comprises an n-decanoyl side chain linked via a three-amino acid chain to the N-terminal tryptophan of a cyclic 10-amino acid peptide. The compound is being developed in a variety of formulations to treat serious infections for which therapeutic options are limited, such as infections caused by bacteria including, but not limited to, methicillin resistant Staphylococcus aureus, vancomycin resistant enterococci, glycopeptide intermediary susceptible Staphylococcus aureus, coagulase-negative staphylococci, and penicillin-resistant Streptococcus pneumoniae. See, e.g., Tally et al., Exp. Opin. Invest. Drugs 8:1223-1238, 1999. The antibiotic action of daptomycin against Gram-positive bacteria has been attributed to its ability to interfere with membrane potential and to inhibit lipoteichoic acid synthesis.

Identification of the genes encoding the proteins involved in the daptomycin biosynthetic pathway, including the daptomycin NRPS, will provide a first step in producing modified Streptomyces roseosporus as well as other host strains which can produce an improved antibiotic (for example, having greater potency); which can produce natural or new antibiotics in increased quantities; or which can produce other peptide products having useful biological properties. Compositions and methods relating to the Streptomyces roseosporus daptomycin biosynthetic gene cluster, including isolated nucleic acids and isolated proteins, are described in U.S. Provisional Application 60/240,879, filed Oct. 17, 2000; 60/272,207, filed Feb. 28, 2001; and 60/310,385, filed Aug. 8, 2001; all of which are hereby incorporated by reference in its entirety.

It would be advantageous, moreover, to identify the genetic and modular organization of the Streptomyces roseosporus daptomycin biosynthetic gene cluster in order to construct full length daptomycin NRPS templates for expression in Streptomyces roseosporus and in heterologous hosts. In particular, it would be advantageous to know whether the daptomycin gene cluster comprises a thioesterase (Te) domain. If so, that Te domain could be isolated and used to catalyze peptide chain termination in new NRPS modules and templates by expression as a fusion or as a free peptide. See, e.g., de Ferra et al., J. Biol. Chem., 272, pp. 25304-25309 (1997); Guenzi et al., J. Biol. Chem., 273, pp. 14403-14410 (1998); and Trauger et al., Nature, 407, pp. 215-218 (2000); each incorporated herein by reference in its entirety. It would also be advantageous to identify other nucleic acid molecules that encode polypeptides involved in daptomycin biosynthesis. These include, without limitation, enzymes involved in attaching a lipid tail to the peptide domain of daptomycin, polypeptides that regulate antibiotic resistance and ABC transporters. Polypeptides that regulate antibiotic resistance and ABC transporters could be used to confer resistance or increase, modify or decrease resistance of a bacteria to daptomycin and related antibiotics. Polypeptides involved in antibiotic resistance would also be useful to determine bacterial mechanisms of resistance, so that daptomycin and related antibiotics can be modified to make them more potent against resistant bacteria.

### SUMMARY OF THE INVENTION

The instant invention addresses these problems by providing a nucleic acid molecule that comprises all or a part of a daptomycin biosynthetic gene cluster, preferably one from S. roseosporus. The nucleic acid molecule may encode DptA, DptBC or DptD or may comprise one or more of the dptA, dptBC or dptD genes from the daptomycin biosynthetic gene cluster of S. roseosporus.

The instant invention also provides nucleic acid molecules encoding a free thioesterase and an integral thioesterase from a daptomycin biosynthetic gene cluster. The nucleic acid molecule may encode DptH or the thioesterase domain from DptD, or may comprise the dptH or dptD gene from the daptomycin biosynthetic gene cluster.

Another object of the invention is to provide a nucleic acid molecule comprising a DNA sequence from a bacterial artificial chromosome comprising a nucleic acid sequence from S. roseosporus. The nucleic acid molecule preferably comprises a S. roseosporus nucleic acid sequence from any one of bacterial artificial chromosome (BAC) clones B12:01G05, B12:06A12, B12:12F06, B12:18H04, B12:20C09 or B12:03A05. (Of these, only B12:03A05 has been deposited; ATCC Deposit No. PTA-3141, deposited Mar. 1, 2001). In a preferred embodiment, the nucleic acid molecule encodes a polypeptide. In another preferred embodiment, the nucleic acid molecule encodes a polypeptide that is involved in daptomycin biosynthesis, such as a dptA, dptBC, dptD, dptE, dptF, dptH, an ABC transporter, or a polypeptide that regulates antibiotic resistance, as described herein.

The invention also provides selectively hybridizing or homologous nucleic acid molecules of the above-described nucleic acid molecules. The invention further provides allelic variants and parts thereof. The invention further provides nucleic acid molecules that comprise one or more expression control sequences controlling the transcription of the above-described nucleic acid molecules. The expression control sequence may be derived from the expression control sequences of the daptomycin biosynthetic gene cluster or may be derived from a heterologous nucleic acid sequence.

In another embodiment, the invention provides a nucleic acid molecule comprising one or more expression control sequences from a gene comprising a nucleic acid sequence that encodes a thioesterase and/or a daptomycin NRPS from the daptomycin biosynthetic gene cluster. Preferably, the nucleic acid molecule comprises a part or all of the expression control sequences of the daptomycin NRPS or dptH.

Another object of the invention is to provide a vector and/or host cell comprising one or more of the above-described nucleic acid molecules. In a preferred embodiment, the vector and/or host cell comprises a nucleic acid molecule encoding all or part of DptA, DptBC, DptD, DptE, DptF and/or DptH, or all or part of a BAC clone described above. A host cell may comprise all or a part of an NRPS or PKS, such as a daptomycin NRPS. The host cell may further comprise one or more thioesterases.

Another object of the invention is to provide a polypeptide derived from the daptomycin biosynthetic gene cluster, preferably a polypeptide from the daptomycin biosynthetic gene cluster of S. roseosporus. The polypeptide may be DptA, DptBC or DptD.

The invention also provides a polypeptide derived from an integral or free thioesterase, preferably one derived from a daptomycin biosynthetic gene cluster of S. roseosporus. In a preferred embodiment, the polypeptide is derived from thioesterase. The polypeptide may be derived from DptH or the thioesterase domain of DptD.

The invention also provides a polypeptide encoded by a nucleic acid molecule of any one of BAC clones B12:01G05, B12:06A12, B12:12F06, B12:18H04, B12:20C09 or B12:03A05. These polypeptides include, among others, enzymes involved in attaching a lipid tail to the peptide domain of daptomycin, polypeptides that regulate antibiotic resistance and ABC transporters.

Another object of the invention is to provide fragments of the polypeptides described above. In one embodiment, the fragment comprises at least one domain or module, as defined herein. In another embodiment, the fragment comprises at least one epitope of the polypeptide.

Another object of the invention is to provide polypeptides that are mutant proteins, fusion proteins, homologous proteins or allelic variants of the daptomycin NRPS polypeptides, thioesterases and polypeptides encoded by the nucleic acid molecules of the BAC clones provided herein.

The invention also provides an antibody that specifically binds to a polypeptide of a daptomycin NRPS, a thioesterase polypeptide of a daptomycin biosynthetic gene cluster or a polypeptide encoded by a nucleic acid molecule from any one of BAC clones B12:01G05, B12:06A12, B12:12F06, B12:18H04, B12:20C09 or B12:03A05. The invention also provides an antibody that can bind to a fragment, polypeptide mutant, a fusion protein, a polypeptide encoded by an allelic variant or a homologous protein of any one of the above-described polypeptides or proteins. The antibodies may be used to detect the presence or amount of a polypeptide of the instant invention or to inhibit or activate an activity of a polypeptide.

Another objective of the instant invention is to provide a method for recombinantly producing a polypeptide using a nucleic acid molecule described herein by introducing a nucleic acid molecule into a host cell and expressing the polypeptide.

The instant invention also provides a method for using the nucleic acid molecules of the instant invention to detect or amplify nucleic acid molecules that have similar or identical nucleic acid sequences compared to the nucleic acid molecules described herein.

The nucleic acid molecules and polypeptides are useful for, for example, the biosynthesis and production of natural products and the engineered biosynthesis of new compounds. The daptomycin NRPS and/or thioesterases may be used to produce daptomycin and other lipopeptides, including both naturally-occurring and novel compounds. The polypeptides may be used in vitro for the production of cyclic or non-cyclic lipopeptides, as well as other compounds produced by non-ribosomal peptide synthesis. Alternatively, a nucleic acid molecule of the invention may be introduced and expressed in a host cell, and the host cell may then be used to produce lipopeptides and other compounds produced by non-ribosomal peptide synthesis.

Another objective of the invention is to provide a novel gene cluster that can produce novel compounds by non-ribosomal peptide synthesis. A novel gene cluster may be obtained by altering nucleotides of the daptomycin biosynthetic gene cluster, particularly by altering nucleotides, domains or modules of the daptomycin NRPS, to make new polypeptides that are involved in non-ribosomal peptide synthesis. In this manner, different amino acids may be incorporated into a peptide produced by non-ribosomal peptide synthesis than the peptide produced by a naturally-occurring polypeptide. The invention also encompasses the compounds produced by the methods described herein.

Another objective of the invention is to provide a computer readable means of storing the nucleic acid and amino acid sequences of the instant invention. The records of the computer readable means can be accessed for reading and display of sequences and for comparison, alignment and ordering of the sequences of the invention to other sequences.

### BRIEF DESCRIPTION OF THE DRAWINGS

FIG. 1 is a schematic diagram of methods in which daptomycin NRPS genes can be manipulated to alter gene expression or expression of the encoded proteins.

FIG. 2A is a schematic diagram of BAC clone B12:03A05. The diagram shows a 90 kb region, referred to as the 90 kb fragment, an approximately 13 kb region, referred to herein as the SP6 fragment, and an approximately 25-28 kb region, referred to herein as the GTC2 fragment. SEQ ID NO: 1 shows the nucleic acid sequence of the 90 kb fragment. SEQ ID NO: 103 shows the nucleic acid sequence of the SP6 fragment. The SP6 fragment flanks the 90 kb fragment at the left. The GTC2 fragment flanks the 90 kb fragment on the right. SEQ ID NO: 104 shows the nucleic acid sequence of the GTC2 fragment.

FIG. 2B shows a schematic diagram of the 90 kb fragment. There are 38 open reading frames (ORFs), which are nucleic acid sequences that encode polypeptides, in the region of the daptomycin biosynthetic gene cluster.

FIG. 2C shows a schematic diagram of the SP6 fragment. There are 9 ORFs in the SP6 fragment. See Table 5 for the amino acid and nucleic acid sequence identifiers for the ORFs of the 90 kb and the SP6 fragment.

FIG. 2D shows a schematic diagram of the GTC2 fragment.

FIG. 3 shows a comparison of the amino acid sequences of DptD (SEQ ID NO: 7) and the calcium dependent antibiotic (CDA) III protein of Streptomyces coelicolor (SEQ ID NO: 164) using the Clustal W program. See Example 3.

FIG. 4 shows a comparison of the amino acid sequences of DptH (SEQ ID NO: 8) and the probable hydrolase (presumed thioesterase) associated with the CDA NRPS of Streptomyces coelicolor (SEQ ID NO: 165) using the Clustal W program. See Example 3.

FIGS. 5A-5C shows an analysis of daptomycin or A21978C lipopeptides produced from the Streptomyces lividans TK64 clone containing the daptomycin biosynthetic gene cluster CBUK138742 (ATCC Deposit PTA-3140, deposited Mar. 1, 2001). FIG. 5A shows an HPLC analysis of the broth of CBUK138742. The lower panel shows a trace plotting the maximum absorbance observed over the range of 200-600 nm for the HPLC eluate against time. The presence of three native lipopeptides, lipopeptides A21978C1 (the C1 lipopeptide), A21978C2 (the C2 lipopeptide) and A21978C3 (the C3 lipopeptide), is indicated by peaks with retention times of 5.61, 5.77 and 5.89 minutes, respectively. The upper panel shows the UV-visible spectra observed for these peaks. FIG. 5B shows an ESI mass spectrum of daptomycin purified from decanoic acid-fed fermentation of Streptomyces lividans TK64 clone containing the daptomycin gene cluster. FIG. 5C shows a 1H NMR spectrum (400 MHz, in d6-DMSO) of daptomycin purified from decanoic acid-fed fermentation of CBUK138742.

FIG. 6 is a diagram of the cloning vector pStreptoBAC V.

FIG. 7 shows a HinDIII digest of BAC clones from the daptomycin biosynthetic gene cluster. Lane 1 shows B12:01G05 (82 kb insert); Lane 2 shows B12:03A05 (120 kb insert); Lane 3 shows B12:06A12 (85 kb insert); Lane 3 shows B12:12FG06 (65 kb insert); Lane 5 shows B12:18H04 (46 kb insert) and Lane 6 shows B12:20C09 (65 kb insert).

FIG. 8 shows a map of some BAC clones that cover approximately 180 to 200 kb of the daptomycin NPRS region in Streptomyces roseosporus.

FIG. 9 is a schematic diagram of the gene structure of an NRPS.

FIG. 10 is a dendrogram showing the adenylation (A) domain similarities for domains that specify Asn and Asp in the daptomycin NRPS and in the CDA NRPS from Streptomyces coelicolor. See Example 5.

FIG. 11 shows the results of an HPLC analysis determining the stereochemistry of Asn. See Example 6.

FIG. 12 is a schematic diagram showing the organization of the daptomycin NRPS.

FIG. 13 shows a 1H NMR spectrum the novel lipopeptide produced as described in Example 12C.

### DETAILED DESCRIPTION OF THE INVENTION

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Sambrook et al. Molecular Cloning: A Laboratory Manual, 3d ed., Cold Spring Harbor Laboratory Press, Cold Spring Harbor, N.Y. (2000); Ausubel et al., Current Protocols in Molecular Biology, Greene Publishing Associates (1992, and Supplements to 2000); Ausubel et al., Short Protocols in Molecular Biology: A Compendium of Methods from Current Protocols in Molecular Biology, 4th ed., Wiley & Sons (1999); Harlow and Lane Antibodies: A Laboratory Manual, Cold Spring Harbor Laboratory Press, Cold Spring Harbor, N.Y. (1990); Harlow and Lane Using Antibodies: A Laboratory Manual, Cold Spring Harbor Laboratory Press, Cold Spring Harbor, N.Y. (1998); and T. Kieser et al., Practical Streptomyces Genetics, John Innes Foundation, Norwich (2000); each of which is incorporated herein by reference in its entirety.

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The term “thioesterase” refers to an enzyme that is capable of catalyzing the cleavage of a thioester bond, which may result in the production of a cyclic or linear molecule.

The term “thioesterase activity” refers to an enzymatic activity of a thioesterase, or a mutein, homologous protein, analog, derivative, fusion protein or fragment thereof, that catalyzes cleavage of a thioester bond. A thioesterase activity includes, e.g., an association and/or dissociation constant, a catalytic rate and a substrate turnover rate. A thioesterase activity of a polypeptide may be the same as one of the thioesterase activities of DptH, the thioesterase domain of DptD, a polypeptide encoded by dptH, a polypeptide encoded by the thioesterase domain of dptD, a polypeptide having an amino acid sequence of the thioesterase domain of SEQ ID NO: 7 or a polypeptide having the amino acid sequence of SEQ ID NO: 8. The thioesterase activity may also different from that of one of the above-described thioesterases; e.g., it may have an increased or decreased catalytic activity, a different association and/or dissociation constant or a different substrate for catalysis. A “decreased” or “increased” thioesterase activity refers to a decreased or increased catalytic activity of the thioesterase, respectively.

A “thioesterase derived from a daptomycin biosynthetic gene cluster” is a thioesterase or thioesterase domain that is encoded by one of the genes of a gene cluster that encodes polypeptides involved in the synthesis of daptomycin. Preferably, the thioesterase is derived from a daptomycin biosynthetic gene cluster from Streptomyces, preferably from a daptomycin biosynthetic gene cluster from S. roseosporus.

A “daptomycin biosynthetic gene cluster” is defined herein as a nucleic acid molecule that encodes a number of polypeptides that are necessary for synthesis of daptomycin in an organism, preferably in a bacterial cell. A daptomycin biosynthetic gene cluster comprises a nucleic acid molecule that encodes at least DptA, DptBC, DptD and DptH, or that encode muteins, homologous proteins, allelic variants or fragments thereof, as well as other nucleic acid sequences that encode other polypeptides required for daptomycin synthesis. Preferably, a daptomycin biosynthetic gene cluster comprises that part of BAC B12:03A05 that permits the synthesis of daptomycin when the part is introduced and expressed in a bacterial cell.

A “daptomycin NRPS” is defined herein as an NRPS that is capable of synthesizing daptomycin in an appropriate bacterial cell. A daptomycin NRPS comprises polypeptide subunits DptA, DptBC and DptD, or muteins, homologous proteins, allelic variants or fragments thereof, that are capable, when expressed in an appropriate cell, of directing the synthesis of daptomycin. A daptomycin NRPS may further comprise DptH and/or other polypeptide, such as DptE or DptF. Preferably, the daptomycin NRPS is derived from the daptomycin biosynthetic gene cluster from Streptomyces, more preferably, the daptomycin NRPS is derived from S. roseosporus. The term “daptomycin NRPS” does not imply that the daptomycin NRPS can be used to synthesize only daptomycin. Rather, as used herein, the term is used solely for the purpose of describing that the NRPS was originally derived from a daptomycin biosynthetic gene cluster. The daptomycin NRPS may be used to synthesize molecules other than daptomycin, as described herein.

A “gene” is defined as a nucleic acid molecule that comprises a nucleic acid sequence that encodes a polypeptide and the expression control sequences that are operably linked to the nucleic acid sequence that encodes the polypeptide. For instance, a gene may comprise a promoter, one or more enhancers, a nucleic acid sequence that encodes a polypeptide, downstream regulatory sequences and, possibly, other nucleic acid sequences involved in regulation of the expression of an RNA.

A nucleic acid molecule or polypeptide is “derived” from a particular species if the nucleic acid molecule or polypeptide has been isolated from the particular species, or if the nucleic acid molecule or polypeptide is homologous to a nucleic acid molecule or polypeptide isolated from a particular species.

The terms “dptA”, “dptBC” and “dptD” refer to nucleic acid molecules that encode subunits of the daptomycin NRPS. In a preferred embodiment, the nucleic acid molecule is derived from Streptomyces, more preferably the nucleic acid molecule is derived from S. roseosporus. In a preferred embodiment, the dptA, dptBC and dptD encode the polypeptides having the amino acid sequences of SEQ ID NOS: 9, 11 and 7, respectively. The terms “dptA”, “dptBC” and “dptD” also refer to allelic variants of these genes, which may be obtained from other species of Streptomyces or from other S. roseosporus strains.

The term “dptH” refers to a gene whose coding domain encodes a thioesterase from a daptomycin biosynthetic gene cluster of S. roseosporus, wherein the naturally-occurring thioesterase is a “free” thioesterase. A free thioesterase is one that is not a functional domain of a larger polypeptide when it is naturally occurring. The dptH gene also encompasses the expression control sequences that are upstream of the coding region of the gene, as discussed below. In one embodiment, the expression control sequences of dptH have the nucleic acid sequence of SEQ ID NO: 5. The term “dptH” also refers to the nucleic acid encoding the polypeptide defined by SEQ ID NO: 8. The term “dptH” also refers to allelic variants of this gene, which may be obtained from other species of Streptomyces or from other S. roseosporus strains.

The term “allelic variant” refers to one of two or more alternative naturally-occurring forms of a gene, wherein each allele possesses a different nucleotide sequence. An allelic variant may encode the same polypeptide or a different one. As used herein, an allele is one that has at least 90% sequence identity, more preferably at least 95%, 96%, 97%, 98% or 99% sequence identity to the reference nucleic acid sequence, and encodes a polypeptide having similar or identical biological properties as the polypeptide encoded by the reference nucleic acid molecule.

The term “polynucleotide” or “nucleic acid molecule” refers to a polymeric form of nucleotides of at least 10 bases in length, either ribonucleotides or deoxynucleotides or a modified form of either type of nucleotide. The term includes single and double stranded forms of DNA. In addition, a polynucleotide may include either or both naturally-occurring and modified nucleotides linked together by naturally-occurring and/or non-naturally occurring nucleotide linkages.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases, or genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature as part of a larger sequence. The term “isolated” or “substantially pure” also can be used in reference to recombinant or cloned DNA isolates, chemically synthesized polynucleotide analogs, or polynucleotide analogs that are biologically synthesized by heterologous systems.

A “part” of a nucleic acid molecule or polynucleotide refers to a nucleic acid molecule that comprises a partial contiguous sequence of at least 14 nucleotides of the reference nucleic acid molecule. Preferably, a part comprises at least 17 or 20 nucleotides of a reference nucleic acid molecule. More preferably, a part comprises at least 25, 30, 35, 40, 50, 60, 70, 80, 90, 100, 200, 300 400, 500 or 1000 nucleotides up to one nucleotide short of a reference nucleic acid molecule. A part of a nucleic acid molecule may comprise no other nucleic acid sequences. Alternatively, a part of a nucleic acid may comprise other nucleic acid sequences from other nucleic acid molecules.

The term “oligonucleotide” refers to a polynucleotide generally comprising a length of 200 nucleotides or fewer. Preferably, oligonucleotides are 10 to 60 nucleotides in length and most preferably 12, 13, 14, 15, 16, 17, 18, 19, 20, 30, 40, 50 or 60 nucleotides in length. Oligonucleotides may be single-stranded, e.g. for use as probes or primers, or may be double-stranded, e.g. for use in the construction of a mutant gene. Oligonucleotides of the invention can be either sense or antisense oligonucleotides. An oligonucleotide can include a label for detection, if desired.

The term “naturally-occurring nucleotide” referred to herein includes naturally-occurring deoxyribonucleotides and ribonucleotides. The term “modified nucleotides” referred to herein includes nucleotides with modified or substituted sugar groups and the like. The term “nucleotide linkages” referred to herein includes nucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference.

Unless specified otherwise, the left hand end of a polynucleotide sequence in sense orientation is the 5′ end and the right hand end of the sequence is the 3′ end. In addition, the left hand direction of a polynucleotide sequence in sense orientation is referred to as the 5′ direction, while the right hand direction of the polynucleotide sequence is referred to as the 3′ direction.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. There are a number of different algorithms known in the art which can be used to measure nucleotide sequence identity. In one embodiment, polynucleotide sequences may be compared using Blast (Altschul et al., J. Mol. Biol. 215: 403-410, 1990). For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences (Pearson, 1990, (herein incorporated by reference). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 50%, more preferably 60% of the nucleotide bases, usually at least about 70%, more usually at least about 80%, preferably at least about 90%, and more preferably at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under selective hybridization conditions. Typically, selective hybridization will occur when there is at least about 55% sequence identity—preferably at least about 65%, more preferably at least about 75%, and most preferably at least about 90%—over a stretch of at least about 14 nucleotides. See, e.g., Kanehisa, 1984, herein incorporated by reference.

Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. “Stringent hybridization conditions” and “stringent wash conditions” in the context of nucleic acid hybridization experiments depend upon a number of different physical parameters. The most important parameters include temperature of hybridization, base composition of the nucleic acids, salt concentration and length of the nucleic acid. One having ordinary skill in the art knows how to vary these parameters to achieve a particular stringency of hybridization.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., supra, page 9.51, hereby incorporated by reference.

The Tₘ for a particular DNA-DNA hybrid can be estimated by the formula:
Tₘ=81.5° C.+16.6(log₁₀[Na⁺])+0.41(fraction G+C)−0.63(% formamide)−(600/l) (i)
where l is the length of the hybrid in base pairs.

The Tₘ for a particular RNA-RNA hybrid can be estimated by the formula:
Tₘ=79.8° C.+18.5(log₁₀[Na⁺])+0.58(fraction G+C)+11.8(fraction G+C)²−0.35(% formamide)−(820/l). (ii)

The Tₘ for a particular RNA-DNA hybrid can be estimated by the formula:
Tₘ=79.8° C.+18.5(log₁₀[Na⁺])+0.58(fraction G+C)+11.8(fraction G+C)²−0.50(% formamide)−(820/l). (iii)

In general, the Tₘ decreases by 1-1.5° C. for each 1% of mismatch between two nucleic acid sequences. Thus, one having ordinary skill in the art can alter hybridization and/or washing conditions to obtain sequences that have higher or lower degrees of sequence identity to the target nucleic acid. For instance, to obtain hybridizing nucleic acids that contain up to 10% mismatch from the target nucleic acid sequence, 10-15° C. would be subtracted from the calculated Tₘ of a perfectly matched hybrid, and then the hybridization and washing temperatures adjusted accordingly. Probe sequences may also hybridize specifically to duplex DNA under certain conditions to form triplex or other higher order DNA complexes. The preparation of such probes and suitable hybridization conditions are well known in the art.

An example of stringent hybridization conditions for hybridization of complementary nucleic acid sequences having more than 100 complementary residues on a filter in a Southern or Northern blot or for screening a library is 50% formamide/6×SSC at 42° C. for at least ten hours, preferably 12-16 hours. Another example of stringent hybridization conditions is 6×SSC at 68° C. without formamide for at least ten hours, preferably 12-16 hours. An example of low stringency hybridization conditions for hybridization of complementary nucleic acid sequences having more than 100 complementary residues on a filter in a Southern or northern blot or for screening a library is 6×SSC at 42° C. for at least ten hours, preferably 12-16 hours. Hybridization conditions to identify nucleic acid sequences that are similar but not identical can be identified by experimentally changing the hybridization temperature from 68° C. to 42° C. while keeping the salt concentration constant (6×SSC), or keeping the hybridization temperature and salt concentration constant (e.g. 42° C. and 6×SSC) and varying the formamide concentration from 50% to 0%. Hybridization buffers may also include blocking agents to lower background. These agents are well-known in the art. See Sambrook et al., supra, pages 8.46 and 9.46-9.58, herein incorporated by reference.

Wash conditions also can be altered to change stringency conditions. An example of stringent wash conditions is a 0.2×SSC wash at 65° C. for 15 minutes (see Sambrook et al., supra, for SSC buffer). Often the high stringency wash is preceded by a low stringency wash to remove excess probe. An exemplary medium stringency wash for duplex DNA of more than 100 base pairs is 1×SSC at 45° C. for 15 minutes. An exemplary low stringency wash for such a duplex is 4×SSC at 40° C. for 15 minutes. In general, signal-to-noise ratio of 2× or higher than that observed for an unrelated probe in the particular hybridization assay indicates detection of a specific hybridization.

As defined herein, nucleic acids that do not hybridize to each other under stringent conditions are still substantially homologous to one another if they encode polypeptides that are substantially identical to each other. This occurs, for example, when a nucleic acid is created synthetically or recombinantly using a high codon degeneracy as permitted by the redundancy of the genetic code.

The polynucleotides of this invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The term “mutated” when applied to nucleic acid sequences means that nucleotides in a nucleic acid sequence may be inserted, deleted or changed compared to a reference nucleic acid sequence. A single alteration may be made at a locus (a point mutation) or multiple nucleotides may be inserted, deleted or changed at a single locus. In addition, one or more alterations may be made at any number of loci within a nucleic acid sequence. In a preferred embodiment, the nucleic acid sequence is the wild type nucleic acid sequence for a thioesterase. The nucleic acid sequence may be mutated by any method known in the art including those mutagenesis techniques described infra.

The term “error-prone PCR” refers to a process for performing PCR under conditions where the copying fidelity of the DNA polymerase is low, such that a high rate of point mutations is obtained along the entire length of the PCR product. See, e.g., Leung et al., Technique, 1, pp. 11-15 (1989) and Caldwell and Joyce PCR Methods Applic., 2, pp. 28-33 (1992).

The term “oligonucleotide-directed mutagenesis” refers to a process which enables the generation of site-specific mutations in any cloned DNA segment of interest. See, e.g., Reidhaar-Olson et al., Science, 241, pp. 53-57 (1988).

The term “assembly PCR” refers to a process which involves the assembly of a PCR product from a mixture of small DNA fragments. A large number of different PCR reactions occur in parallel in the same vial, with the products of one reaction priming the products of another reaction.

The term “sexual PCR mutagenesis” or “DNA shuffling” refers to a method of error-prone PCR coupled with forced homologous recombination between DNA molecules of different but highly related DNA sequence in vitro, caused by random fragmentation of the DNA molecule based on sequence homology, followed by fixation of the crossover by primer extension in an error-prone PCR reaction. See, e.g., Stemmer, Proc. Natl. Acad. Sci. U.S.A., 91, pp. 10747-10751 (1994). DNA shuffling can be carried out between several related genes (“Family shuffling”).

The term “in vivo mutagenesis” refers to a process of generating random mutations in any cloned DNA of interest which involves the propagation of the DNA in a strain of bacteria such as E. coli that carries mutations in one or more of the DNA repair pathways. These “mutator” strains have a higher random mutation rate than that of a wild-type parent. Propagating the DNA in a mutator strain will eventually generate random mutations within the DNA.

The term “cassette mutagenesis” refers to any process for replacing a small region of a double-stranded DNA molecule with a synthetic oligonucleotide “cassette” that differs from the native sequence. The oligonucleotide often contains completely and/or partially randomized native sequence.

The term “recursive ensemble mutagenesis” refers to an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. See, e.g., Arkin and Youvan, Proc. Natl. Acad. Sci. U.S.A., 89, pp. 7811-7815 (1992).

The term “exponential ensemble mutagenesis” refers to a process for generating combinatorial libraries with a high percentage of unique and functional mutants, wherein small groups of residues are randomized in parallel to identify, at each altered position, amino acids which lead to functional proteins. See, e.g., Delegrave and Youvan, Biotechnol. Res., 11, pp. 1548-1552 (1993); and random and site-directed mutagenesis, Arnold, Curr. Opin. Biotechnol., 4, pp. 450-455 (1993). Each of the references mentioned above are hereby incorporated by reference in its entirety.

“Operatively linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome. Viral vectors that infect bacterial cells are referred to as bacteriophages. Certain vectors are capable of autonomous replication in a host cell into which they are introduced (e.g., bacterial vectors having a bacterial origin of replication). Other vectors can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include other forms of expression vectors that serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “polypeptide” encompasses both naturally-occurring and non-naturally-occurring proteins and polypeptides, polypeptide fragments and polypeptide mutants, derivatives and analogs. As used herein, a polypeptide comprises at least six amino acids, preferably at least 8, 10, 12, 15, 20, 25 or 30 amino acids, and more preferably the polypeptide is the full length of the naturally-occurring polypeptide. A polypeptide may be monomeric or polymeric. Further, a polypeptide may comprise a number of different modules within a single polypeptide each of which has one or more distinct activities. A preferred polypeptide in accordance with the invention comprises a thioesterase derived from the daptomycin biosynthetic gene cluster, as well as a fragment, mutant, analog and derivative thereof.

The term “isolated protein” or “isolated polypeptide” is a protein or polypeptide that by virtue of its origin or source of derivation (1) is not associated with naturally associated components that accompany it in its native state, (2) is free of other proteins from the same species (3) is expressed by a cell from a different species, or (4) does not occur in nature. Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art.

A protein or polypeptide is “substantially pure,” “substantially homogeneous” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A “derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See Ausubel et al., 1992, hereby incorporated by reference.

The term “fusion protein” refers to polypeptides comprising polypeptides or fragments coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

The term “non-peptide analog” refers to a compound with properties that are analogous to those of a reference polypeptide. A non-peptide compound may also be termed a “peptide mimetic” or a “peptidomimetic.” See, e.g., Fauchere, J. Adv. Drug Res. 15:29 (1986); Veber and Freidinger Trends Neurosci. p. 392 (1985); and Evans et al. J. Med. Chem. 30:1229 (1987), which are incorporated herein by reference. Such compounds are often developed with the aid of computerized molecular modeling. Peptide mimetics that are structurally similar to useful peptides may be used to produce an equivalent effect. Generally, peptidomimetics are structurally similar to a paradigm polypeptide (i.e., a polypeptide that has a desired biochemical property or pharmacological activity), such as a thioesterase, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch, Annu. Rev. Biochem. 61:387 (1992), incorporated herein by reference); for example, by adding internal cysteine residues capable of forming intramolecular disulfide bridges which cyclize the peptide.

A “polypeptide mutant” or “mutein” refers to a polypeptide whose sequence contains substitutions, insertions or deletions of one or more amino acids compared to the amino acid sequence of a native or wild type protein. A mutein may have one or more amino acid point substitutions, in which a single amino acid at a position has been changed to another amino acid, one or more insertions and/or deletions, in which one or more amino acids are inserted or deleted, respectively, in the sequence of the naturally-occurring protein, and/or truncations of the amino acid sequence at either or both the amino or carboxy termini. Further, a mutein may have the same or different biological activity as the naturally-occurring protein. For instance, a mutein may have an increased or decreased biological activity. In a preferred embodiment of the present invention, a mutein has the same or increased thioesterase activity as a naturally-occurring thioesterase. A mutein has at least 50%, 60% or 70% sequence homology to the wild type protein, more preferred are muteins having at least 80%, 85% or 90% sequence homology to the wild type protein, even more preferred are muteins exhibiting at least 95%, 96%, 97%, 98% or 99% sequence identity. Sequence homology may be measured by any common sequence analysis algorithm, such as Gap or Bestfit, using default parameters.

Preferred amino acid substitutions are those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such derivatives, analogs, fusion proteins and muteins. Single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the polypeptide notation used herein, the lefthand direction is the amino terminal direction and the right hand direction is the carboxy-terminal direction, in accordance with standard usage and convention.

A protein has “homology” or is “homologous” to a protein from another organism if the encoded amino acid sequence of the protein has a similar sequence to the encoded amino acid sequence of a protein of a different organism. Alternatively, a protein may have homology or be homologous to another protein if the two proteins have similar amino acid sequences. Although two proteins are said to be “homologous,” this does not imply that there is necessarily an evolutionary relationship between the proteins. Instead, the term “homologous” is defined to mean that the two proteins have similar amino acid sequences. In a preferred embodiment, a homologous protein is one that exhibits at least 50%, 60% or 70% sequence identity to the wild type protein, preferred are homologous proteins that exhibit at least 80%, 85%, 90%, 95%, 96%, 97%, 98% or 99% sequence identity. In addition, although in many cases proteins with similar amino acid sequences will have similar functions, the term “homologous” does not imply that the proteins must be functionally similar to each other.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, herein incorporated by reference).

The following six groups each contain amino acids that are conservative substitutions for one another:
1. Serine (S), Threonine (T);
2. Aspartic Acid (D), Glutamic Acid (E);
3. Asparagine (N), Glutamine (Q);
4. Arginine (R), Lysine (K);
5. Isoleucine (I), Leucine (L), Methionine (M), Alanine (A), Valine (V), and
6. Phenylalanine (F), Tyrosine (Y), Tryptophan (W).

Sequence homology for polypeptides, which is also referred to as sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A preferred algorithm when comparing a polypeptide sequence to a database containing a large number of sequences from different organisms is the computer program BLAST, especially blastp, tblastn or BlastX. See Altschul et al. Nucleic Acids Res. 25:3389-3402 (1997), herein incorporated by reference. BlastX, which compares a translated nucleotide sequence to a protein database, may be performed online through the servers located at the National Center for Biotechnology Information. Preferred parameters for blastp, which compares a protein sequence to a protein database are:

Expectation value: 10 (default)

Filter: seg (default)

Cost to open a gap: 11 (default)

Cost to extend a gap: 1 (default

Max. alignments: 100 (default)

Word size: 11 (default)

No. of descriptions: 100 (default)

Penalty Matrix: BLOSUM62

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, herein incorporated by reference). For example, percent sequence identity between amino acid sequences can be determined using FASTA with its default parameters (a word size of 2 and the PAM250 scoring matrix), as provided in GCG Version 6.1, herein incorporated by reference.

An “antibody” refers to an intact immunoglobulin, or to an antigen-binding portion thereof that competes with the intact antibody for antigen-specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. An Fab fragment is a monovalent fragment consisting of the VL, VH, CL and CH1 domains; a F(ab′)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain.

A single-chain antibody (scFv) is an antibody in which a VL and VH regions are paired to form a monovalent molecules via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al., Proc. Natl. Acad. Sci. USA 90:6444-6448, 1993, and Poljak et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature.

A “neutralizing antibody” or “an inhibitory antibody” is an antibody that inhibits the activity of a polypeptide or blocks the binding of a polypeptide to a ligand that normally binds to it. For example, a neutralizing anti-thioesterase antibody may be one that blocks the activity of the thioesterase. An “activating antibody” is an antibody that increases the activity of a polypeptide. For example, an activating anti-thioesterase antibody is one that increases the activity of a thioesterase.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≦1 μM, preferably ≦100 nM and most preferably ≦10 nM.

The term patient includes human and veterinary subjects.

Throughout this specification and claims, the word “comprise,” or variations such as “comprises” or “comprising,” will be understood to imply the inclusion of a stated integer or group of integers but not the exclusion of any other integer or group of integers.

Nucleic Acid Molecules, Regulatory Sequences, Vectors, Host Cells and Recombinant Methods of Making Polypeptides

Nucleic Acid Molecules

In one aspect, the present invention provides a nucleic acid molecule encoding a thioesterase or a daptomycin NRPS or a subunit thereof. In one embodiment, the nucleic acid molecule encodes one or more of DptA, DptBC or DptD. In a preferred embodiment, the nucleic acid molecules encodes a polypeptide comprising any one of the amino acid sequences of SEQ ID NOS: 9, 11 or 7. In another preferred embodiment, the nucleic acid molecule comprises dptA, dptBC and/or dptD. In a further preferred embodiment, the nucleic acid molecule comprises a nucleic acid sequence comprising any one of SEQ ID NOS: 10, 12 or 3.

In another embodiment, the nucleic acid molecule encodes a thioesterase that is derived from a daptomycin biosynthetic gene cluster. In a preferred embodiment, the nucleic acid molecule encodes a thioesterase derived from a daptomycin biosynthetic gene cluster that is a free thioesterase or is an integral thioesterase. In another preferred embodiment, the nucleic acid molecule encodes DptH or the thioesterase domain of DptD. In a more preferred embodiment, the nucleic acid molecule encodes a polypeptide comprising an amino acid sequence of the thioesterase domain of SEQ ID NO: 7 or has the amino acid sequence of SEQ ID NO: 8. In another embodiment, the nucleic acid molecule comprises the thioesterase-encoding domain of dptD or dptH from the daptomycin biosynthetic gene cluster. In another preferred embodiment, the nucleic acid molecule comprises a nucleic acid sequence of SEQ ID NO: 6 or of SEQ ID NO: 3, or the region comprising the thioesterase-encoding portion thereof. In another embodiment, the nucleic acid molecule also encodes a daptomycin NRPS or a subunit thereof. See Examples 1-6 regarding the isolation and identification of dptA, dptBC, dptD and dptH and other genes of the daptomycin biosynthetic gene cluster.

In another embodiment, the nucleic acid molecule encodes an acyl CoA ligase. In a preferred embodiment, the nucleic acid molecule encodes DptE, preferably a nucleic acid molecule encoding SEQ ID NO: 15. In a more preferred embodiment, the nucleic acid molecule comprises dptE. In an even more preferred embodiment, the nucleic acid molecule comprises SEQ ID NO: 16. In another embodiment, the nucleic acid molecule encodes an acyl transferase. In a preferred embodiment, the nucleic acid molecule encodes DptF, preferably a nucleic acid molecule encoding SEQ ID NO: 17. In a more preferred embodiment, the nucleic acid molecule comprises dptF. In an even more preferred embodiment, the nucleic acid molecule comprises SEQ ID NO: 18.

Another embodiment of the invention provides a nucleic acid molecule comprising a DNA sequence from a bacterial artificial chromosome (BAC) comprising nucleic acid sequences from S. roseosporus. In a preferred embodiment, the nucleic acid molecule comprises a S. roseosporus nucleic acid sequence from any one of BAC clones B12:01G05, B12:06A12, B12:12F06, B12:18H04, B12:20C09 or B12:03A05. In a preferred embodiment, the nucleic acid molecule comprises a S. roseosporus nucleic acid sequence from B12:03A05 (ATCC Deposit PTA-3140, deposited Mar. 1, 2001). The nucleic acid molecule may comprise the entire S. roseosporus nucleic acid sequence in the BAC clone or may comprise a part thereof. In a preferred embodiment, the part is a nucleic acid molecule that comprises at least one nucleic acid sequence that can encode a polypeptide, preferably a full-length polypeptide, i.e., a nucleic acid molecule that encodes a polypeptide from its start codon to its stop codon. In one preferred embodiment, the part comprises a nucleic acid molecule encoding a polypeptide involved in daptomycin biosynthesis, such as, without limitation, dptA, dptBC, dptD, dptE, dptF or dptH.

In another embodiment, a part from the BAC clone is a nucleic acid molecule comprising a nucleic acid sequence encoding a polypeptide selected from SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136. In another embodiment, the part from the BAC clone is a nucleic acid molecule comprising a nucleic acid sequence selected from SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135.

The polypeptides having the amino acid sequences of SEQ ID NOS: 110 and 112 are regulators of daptomycin biosynthesis. Multiple regulators are needed for the function of biosynthetic pathways in Streptomyces (Bate et al. Chem. Biol., 6, 617-624, 1999; Baltz, Bioprocess Technol. 22, 308-381, 1995). For example, the biosynthetic pathway for bialaphos in S. hygroscopicus contains a gene, bprA, which has a positive regulatory role in antibiotic production (Raibaud et al., J. Bacteriol. 173, 4454-4463, 1991). It has also been shown that increases of antibiotic production can be achieved by increasing copy number of positive regulator genes in a variety of producing strains (e.g. S. lividans, Vogtli et al., Mol. Microbiol. 14, 643-653, 1994; in S. argillaceus, Lombo et al., J. Bacteriol. 181, 642-647, 1999 and in S. peucetius, Otten et al., Microbiology 146, 1457-1468, 2000). The regulatory activator polypeptide of SEQ ID NO: 110, which is encoded by the nucleic acid molecule having the nucleotide sequence of SEQ ID NO: 109, shares identities and similarities not only with brpA and with other regulatory proteins found in streptomyces but also with the luxR-family proteins involved in quorum sensing. All of these are DNA-binding proteins in the family of two-component transcriptional activators (Kenney, Curr. Opin. Microbiol. 5, 135-141, 2002). Thus, the regulatory activator polypeptide of SEQ ID NO: 110 can be used to augment the yield of daptomycin in S. roseosporus. The regulatory activator gene or a biologically active portion thereof can be cloned into an integrative or autonomously replicating expression vector and reintroduced into one or more neutral sites in one or more copies in S. roseosporus. The transgenic strain may be fermented and analyzed for daptomycin production as described in Example 9 and could be used to produce a larger amount of daptomycin that the wildtype strain.

The polypeptide having the amino acid sequence of SEQ ID NO: 112, which is encoded by the nucleotide sequence of SEQ ID NO: 111, shares significant amounts of identities and similarities with a putative DeoR-family transcriptional regulator from Streptomyces coelicolor as well as a variety of catabolite repressors (LacI, rbsR, malR, REG1). These proteins bind to the promoter regions to prevent the transcription (Zeng and Saxild, J. Bacteriol. 181, 1719-1729, 1999; Oskouian and Stewart, J. Bacteriol. 172, 3804-3812, 1990). Thus, this gene is a negative regulator of daptomycin biosynthesis. Therefore, disruption or deletion of this negative regulator gene or inhibition of its protein product should lead to constitutive expression of daptomycin and/or enhanced yield. In another embodiment, one may delete the negative regulatory gene and insert multiple copies of the positive regulatory gene to increase daptomycin production even more.

The polypeptides having amino acids sequences of SEQ ID NOS: 19, 21, 29, 45, 47, 49, 63, 67, 75 and 77 (nucleic acid sequences of SEQ ID NOS: 20, 22, 30, 46, 48, 50, 64, 68, 76 or 78) are ABC transporters. Some of the polypeptides are pump-like polypeptides with Walker motifs while others are polypeptides that have a role in metal scavenging, e.g., iron or manganese transport (see Tables 6 and 7). The nucleic acid molecule comprising SEQ ID NO: 76 encodes an ATP-binding component of an ABC transporter system, as determined by its sequence similarity to ORF1 of (AAD44229.1) of S. rochei and the S. peucetius DrrA (P32010) genes. The encoded polypeptide has both a Walker A and a Walker B motif. Further, its synthesis appears to be translationally coupled to that of a nucleic acid molecule comprising SEQ ID NO: 78, which encodes a DrrB-like polypeptide, as determined by its sequence similar to the S. peuticeus DrrB product (AAA74718.1), encoding the integral membrane component. The polypeptide having an amino acid sequence of SEQ ID NO: 21 is a StrV homolog, while the polypeptide having an amino acid sequence of SEQ ID NO: 19 is a StrW homolog. See, e.g., Beyer et al., 1996, supra. The StrV homolog has both Walker motifs, while the StrW homolog has only a Walker B motif. Both nucleic acid sequences encoding the polypeptide are on the complementary strand and appear to be translationally regulated. They have S. coelicolor homologs, G8A.01 and G8A.02 (emb| CAB88931, CAB88932). See Tables 6 and 7.

In another aspect, a part of the BAC clone is a nucleic acid molecule comprising a nucleic acid sequence encoding an oxidoreductase; a dehydrogenase; a transcriptional regulator involved in antibiotic resistance; NovABC-related polypeptides, which are involved in the biosynthesis of novobiocin, an antimicrobial agent; a monooxygenase; an acyl CoA thioesterase; a DNA helicase; a DNA ligase; a hydrolase; a thermostable neutral protease; ABC transporters that may be useful in the transport of daptomycin; a spo VK-like protein involved in endospore formation; a serine protease; and an FtsK/SpoIIIE-like protein involved in DNA segregation during septation and spore formation. These nucleic acid molecules and encoded polypeptides may be useful in daptomycin biosynthesis; e.g., the acyl CoA thioesterase may be useful for the reasons provided above for thioesterases and may also be important in the addition of the lipid tail to the peptide domain of daptomycin. These nucleic acid molecules encoding enzymes are also useful because they may be used in the same way as other oxidoreductases, dehydrogenases, monooxygenases, hydrolases, serine or neutral proteases, DNA helicases or DNA ligases are used in the art. Notably, the transcriptional regulator can be mutated using well-known methods to increase or decrease daptomycin or other antibiotic resistance. The nucleic acid molecules encoding NovABC-related polypeptides may be used in the same way as NovABC is used in the art, e.g., to produce novobiocin or related antimicrobial agents. The polypeptides having the above-described activity comprise the amino acid sequences of SEQ ID NOS: 23, 25, 27, 29, 33, 35, 37, 91, 93, 97, 99, 104, 108, 114, 116, 118, 120, 130, 132, 134 and 136 and are encoded by nucleic acid sequences of SEQ ID NOS: 24, 26, 28, 30, 34, 36, 38, 92, 94, 98, 100, 105, 107, 113, 115, 117, 119, 129, 131, 133 and 135.

In another aspect, a part of the BAC clone is a nucleic acid molecule that encodes a polypeptide that does not have a defined function but which is highly homologous to nucleic acid molecules and polypeptides from other Streptomyces. These nucleic acid molecules (SEQ ID NOS: 62, 66, 70, 80, 82, 84, 86, 88, 96, 102, 121, 123, 125 and 127), the polypeptides they encode (SEQ ID NOS: 61, 65, 69, 79, 81, 83, 85, 87, 95, 101, 122, 124, 126 and 128) and antibodies to the polypeptides may be used to identify other Streptomyces species using standard molecular biological and protein chemistry techniques (e.g., PCR, RT-PCR, Southern blotting, northern blotting, ELISAs, radioimmunoassays or western blotting), which is useful, e.g., in microbiological testing or forensics. In another embodiment, a part of the BAC clone is a nucleic acid molecule that encodes a polypeptide that does not have a defined function and is not highly homologous to a nucleic acid molecule or polypeptide from another species. These nucleic acid molecules (SEQ ID NOS: 32, 40, 42, 44, 52, 54, 56, 58, 60, 72 and 74) are nevertheless useful because they are close to the daptomycin biosynthetic gene cluster, and as such, they can be used to identify nucleic acid molecules that encode all or a part of the daptomycin biosynthetic gene cluster. Parts of the BAC clone that do not encode a polypeptide are useful for the same reasons. Further, the polypeptides having the amino acid sequence of SEQ ID NOS: 31, 39, 41, 43, 51, 53, 55, 57, 59, 71 and 73 can be used to make antibodies that can be used to identify S. roseosporus. Because the polypeptides are not highly homologous to any other species, the antibodies would likely be highly specific for S. roseosporus.

In another aspect, the invention provides a nucleic acid molecule that selectively hybridizes to a nucleic acid molecule as described above. In a preferred embodiment, the invention provides a nucleic acid molecule that selectively hybridizes to a nucleic acid molecule that encodes DptA, DptBC, DptD or DptH. In another preferred embodiment, the invention provides a nucleic acid molecules that selectively hybridizes to a nucleic acid molecule that encodes SEQ ID NOS: 9, 11, 7 or 8. In an even more preferred embodiment, the invention provides a nucleic acid molecule that selectively hybridizes to a nucleic acid molecule comprising the nucleic acid sequence of dptA, dptBC, dptD or dptH. In another preferred embodiment, the invention provides a nucleic acid molecule that selectively hybridizes to a nucleic acid molecule comprising the nucleic acid sequence SEQ ID NOS: 10, 12, 3 or 6. The invention also provides a nucleic acid molecule that selectively hybridizes to a nucleic acid molecule comprising an S. roseosporus nucleic acid sequence from any one of BAC clones B12:01G05, B12:06A12, B12:12F06, B12:18H04, B12:20C09 or B12:03A05, preferably that from B12:03A05. In a preferred embodiment, the invention provides a nucleic acid molecule that selectively hybridizes to a nucleic acid molecule encoding SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or to a nucleic acid molecule comprising the nucleic acid sequence SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135. The selective hybridization of any of the above-described nucleic acid sequences may be performed under low stringency hybridization conditions. In a preferred embodiment, the selective hybridization is performed under high stringency hybridization conditions. In a preferred embodiment of the invention, the hybridizing nucleic acid molecule may be used to recombinantly express a polypeptide of the invention.

In another aspect, the invention provides a nucleic acid molecule that is homologous to a nucleic acid encoding a daptomycin NRPS or subunit thereof, a thioesterase from a daptomycin biosynthetic gene cluster, or a nucleic acid molecule comprising an S. roseosporus nucleic acid sequence from any one of BAC clones B12:01G05, B12:06A12, B12:12F06, B12:18H04, B12:20C09 or, preferably, B12:03A05. The invention provides a nucleic acid molecule homologous to a nucleic acid molecule encoding DptA, DptBC, DptD or DptH. In one embodiment, the nucleic acid molecule is homologous to a nucleic acid molecule encoding a polypeptide having an amino acid sequence of SEQ ID NOS: 9, 11, 7 or 8. In a preferred embodiment, the nucleic acid molecule is homologous to any one or more of dptA, dptBC or dptD. In another embodiment, the nucleic acid molecule is homologous to a thioesterase encoded by the thioesterase domain of dptD or by dptH. In a more preferred embodiment, the nucleic acid molecule is homologous to a nucleic acid molecule having a nucleic acid sequence of SEQ ID NOS: 10, 12, 3 or 6. In another preferred embodiment, the invention provides a nucleic acid molecule that is homologous to a nucleic acid molecule encoding SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or to a nucleic acid molecule comprising the nucleic acid sequence SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135. In a preferred embodiment, a homologous nucleic acid molecule is one that has at least 60%, 70%, 80% or 85% sequence identity with a nucleic acid molecule described herein. In a more preferred embodiment, the homologous nucleic acid molecule is one that has at least 90%, 95%, 97%, 98% or 99% sequence identity with a nucleic acid molecule described herein. Further, in one embodiment, a homologous nucleic acid molecule is homologous over its entire length to a nucleic acid molecule encoding a daptomycin NRPS or subunit thereof, a thioesterase, or nucleic acid molecule that encodes a polypeptide as described herein. In another embodiment, a homologous nucleic acid molecule is homologous over only a part of its length to a nucleic acid molecule described herein, wherein the part is at least 50 nucleotides of the nucleic acid molecule, preferably at least 100 nucleotides, more preferably at least 200 nucleotides, even more preferably at least 300 nucleotides.

In another embodiment, the invention provides a nucleic acid that is an allelic variant of a gene encoding a daptomycin NRPS or subunit thereof, a thioesterase from a daptomycin biosynthetic gene cluster, or a nucleic acid molecule comprising an S. roseosporus nucleic acid sequence from any one of BAC clones B12:01G05, B12:06A12, B12:12F06, B12:18H04, B12:20C09 or B12:03A05. In a preferred embodiment, the invention provides a nucleic acid that is an allelic variant of dptA, dptBC, dptD or dptH. In an even more preferred embodiment, the allelic variant is a variant of a gene, wherein the gene encodes DptA, DptBC, DptD or DptH. In another preferred embodiment, the allelic variant is a variant of a gene that encodes a polypeptide comprising an amino acid sequence of SEQ ID NOS: 9, 11, 7 or 8. In a yet more preferred embodiment, the allelic variant is a variant of a gene, wherein the gene has the nucleic acid sequence of SEQ ID NOS: 10, 12, 3 or 6. An allelic variant of dptH or the thioesterase of dptD preferably encodes a thioesterase with the same or similar enzymatic activity compared to that of the polypeptide having the amino acid sequence of the thioesterase domain of SEQ ID NO: 7 or has the amino acid sequence of SEQ ID NO: 8. An allelic variant of dptA, dptBC or dptD preferably encodes a polypeptide having the same activity as the daptomycin NRPS having the amino acid sequences of SEQ ID NOS: 9, 11 or 7, respectively. In another embodiment, the invention provides an allelic variant of a nucleic acid molecule that encodes SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or to a nucleic acid molecule comprising the nucleic acid sequence SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135. In a preferred embodiment, the allelic variant encodes a polypeptide having the same biological activity of the polypeptide; e.g., it encodes a polypeptide having ABC-transporter activity.

A further object of the invention is to provide a nucleic acid molecule that comprises a part of a nucleic acid sequence of the instant invention. The invention provides a part of a nucleic acid molecule encoding a daptomycin NRPS, a subunit thereof, a thioesterase from a daptomycin biosynthetic gene cluster, or a part of a nucleic acid molecule that comprises an S. roseosporus nucleic acid sequence from any one of BAC clones B12:01G05, B12:06A12, B12:12F06, B12:18H04, B12:20C09 or, preferably, B12:03A05. The invention also provides a part of a selectively-hybridizing or homologous nucleic acid molecule, as described above. The invention provides a part of an allelic variant of a nucleic acid molecule, as described above. A part comprises at least 10 nucleotides, more preferably at least 15, 20, 25, 30, 35, 40, 50, 100, 150, 200, 250 or 300 nucleotides. The maximum size of a nucleic acid part is one nucleotide shorter than the entire nucleic acid molecule, if the nucleic acid molecule encodes more than one gene, or is one nucleotide shorter than the nucleic acid molecule encoding the full-length protein, if the nucleic acid molecule encodes a single polypeptide.

In another aspect, the hybridizing or homologous nucleic acid molecule, the allelic variant, or the part of the nucleic acid molecule encodes a polypeptide that has the same biological activity as the native (wild-type) polypeptide.

In another aspect, the invention provides a nucleic acid molecule that encodes a fusion protein, a homologous protein, a polypeptide fragment, a mutein or a polypeptide analog, as described below.

A nucleic acid molecule of this invention may encode a single polypeptide or multiple polypeptides. In one embodiment, the invention provides a nucleic acid molecule that encodes multiple, translationally coupled polypeptides, e.g., a nucleic acid molecule that encodes DptA, DptBC and DptD. The invention also provides a nucleic acid molecule that encodes a single polypeptide derived from S. roseosporus, e.g., DptA, DptBC or DptD, or a polypeptide fragment, mutein, fusion protein, polypeptide analog or homologous protein thereof. The invention also provides nucleic acid sequences, such as expression control sequences, that are not associated with other S. roseosporus sequences.

In certain embodiments, the nucleic acid molecules of this invention may not include any one or more of the plasmids, cosmids designated, pRHB153, pRHB157, pRHB159, pRHB160, pRHB161, pRHB162, pRHB166, pRHB168, pRHB169, pRHB170, pRHB172, pRHB173, pRHB174, pRHB599, pRHB602, pRHB603, pRHB613, pRHB614, pRHB680, pRHB678 or pRHB588 by McHenney et al., J. Bacteriol. 180: 143-151 (1998), herein incorporated by reference in its entirety to the extent any of those plasmids or cosmids are part of the prior art and fall within the scope of any specific claim made in this application. Further analysis performed has indicated that the location and orientation of some of the daptomycin inserts in plasmids or cosmids recited in McHenney et al., supra, are incorrect.

Expression Control Sequences

In another embodiment, the invention provides a nucleic acid molecule comprising one or more expression control sequences from a gene comprising a nucleic acid sequence that encodes a thioesterase or daptomycin NRPS from the daptomycin biosynthetic gene cluster. In a preferred embodiment, the nucleic acid molecule comprises a part or all of the expression control sequences of the daptomycin NRPS or dptH. In a yet more preferred embodiment, the nucleic acid molecule comprises all or a part of SEQ ID NO: 2 or SEQ ID NO: 5. In another preferred embodiment, the nucleic acid molecule comprises an expression control sequence from an S. roseosporus nucleic acid sequence from any one of BAC clones B12:01G05, B12:06A12, B12:12F06, B12:18H04, B12:20C09 or, preferably, B12:03A05. Without wishing to be bound by any theory, it is thought that the nucleic acid sequence upstream of dptA in the daptomycin biosynthetic gene cluster (SEQ ID NO: 2) comprises the native expression control sequences for dptA, dptBC and dptD. Further, it is thought that a single transcript for dptA, dptBC and dptD is generated and that expression of DptA, DptBC and DptD are translationally coupled.

In a preferred embodiment, the entire expression control sequence of a gene comprising a nucleic acid sequence that encodes a daptomycin NRPS and/or a thioesterase from the daptomycin biosynthetic gene cluster is used to control transcription. In another embodiment, only a part of the expression control sequence of a gene comprising a nucleic acid sequence that encodes a daptomycin NRPS and/or a thioesterase from the daptomycin biosynthetic gene cluster is used to control transcription. One having ordinary skill in the art may determine which part(s) of the gene to use to control transcription using methods known in the art. For instance, one may ligate a nucleic acid sequence comprising all or a part of an expression control sequence of a daptomycin NRPS and/or a thioesterase gene into a vector comprising a reporter gene. Examples of such reporter genes include, without limitation, chloramphenicol acetyltransferase (CAT), luciferase, green fluorescent protein, β-galactosidase and the like. The nucleic acid molecule comprising the expression control sequence is ligated into the vector such that it can act as a promoter or enhancer of the reporter gene. The vector is introduced into a host cell and expression is induced. Then, one may assay for the production of the reporter gene product to determine if the part(s) of the expression control sequence is sufficient to activate or regulate transcription. Methods of determining whether a nucleic acid sequence is sufficient to regulate transcription are routine and well-known in the art. See, e.g., Ausubel et al., supra.

A nucleic acid molecule comprising all or a part of an expression control sequence described herein, or multiple copies of these expression control sequences or parts thereof, may be operatively linked to a second nucleic acid molecule to regulate the transcription of the second nucleic acid molecule. In one embodiment, the invention provides a nucleic acid molecule comprising the expression control sequences operatively linked to a heterologous nucleic acid molecule, such as a nucleic acid molecule that encodes a polypeptide not usually expressed by S. roseosporus. In another preferred embodiment, the nucleic acid molecule comprising the expression control sequences is inserted into a vector, preferably a bacterial vector. In a more preferred embodiment, the vector is introduced into a bacterial host cell, more preferably into a Streptomyces or E. coli, and even more preferably into a S. roseosporus, S. lividans or S. fradiae host cell.

The invention also provides a nucleic acid sequence comprising the expression control sequence from S. roseosporus as described herein operatively linked to a nucleic acid sequence encoding a polypeptide involved in a daptomycin NRPS, a thioesterase derived from the daptomycin biosynthetic gene cluster, or a nucleic acid molecule from a BAC clone or part there as described herein. The expression control sequence may be operatively linked to a nucleic acid molecule encoding DptA, DptBC, DptD or DptH, to a nucleic acid molecule encoding a polypeptide derived from the S. roseosporus sequences from a BAC clone of the invention, preferably B12:03A05, or to a nucleic acid molecule encoding a fragment, homologous protein, mutein, analog, derivative or fusion protein thereof. The expression control sequence may be operatively linked to a nucleic acid sequence encoding a polypeptide comprising an amino acid sequence of SEQ ID NOS: 9, 11, 7 or 8, or to a fragment thereof. Preferably, the expression control sequence is operatively linked to the coding region of one or more of dptA, dptBC, dptD or dptH. In a more preferred embodiment, the expression control sequence is operatively linked to a nucleic acid sequence selected from SEQ ID NOS: 10, 12, 3 or 6, or to a part thereof. The invention also provides an expression control sequence operatively linked to the coding region of a polypeptide comprising an amino acid sequence SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or to a nucleic acid molecule comprising the nucleic acid sequence SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135.

In another embodiment, the invention provides a nucleic acid molecule comprising one or more expression control sequences that directs the transcription of a nucleic acid molecule encoding a daptomycin NRPS, a subunit, module or domain thereof, a thioesterase, or a nucleic acid molecule encoding a polypeptide derived from the S. roseosporus sequences from a BAC clone of the invention, wherein the expression control sequence(s) are not derived from a daptomycin biosynthetic gene cluster. Examples of suitable expression control sequences are provided infra.

Expression Vectors, Host Cells and Recombinant Methods of Producing Polypeptides

Nucleic acid sequences may be expressed by operatively linking them to an expression control sequence in an appropriate expression vector and employing that expression vector to transform an appropriate unicellular host. Expression control sequences are sequences which control the transcription, post-transcriptional events and translation of nucleic acid sequences. Such operative linking of a nucleic sequence of this invention to an expression control sequence, of course, includes, if not already part of the nucleic acid sequence, the provision of a translation initiation codon, ATG or GTG, in the correct reading frame upstream of the nucleic acid sequence.

A wide variety of host/expression vector combinations may be employed in expressing the nucleic acid sequences of this invention. Useful expression vectors, for example, may consist of segments of chromosomal, non-chromosomal and synthetic nucleic acid sequences.

In a preferred embodiment, bacterial host cells are used to express the nucleic acid molecules of the instant invention. Useful expression vectors for bacterial hosts include bacterial plasmids, such as those from E. coli or Streptomyces, including pBluescript, pGEX-2T, pUC vectors, col E1, pCR1, pBR322, pMB9 and their derivatives, wider host range plasmids, such as RP4, phage DNAs, e.g., the numerous derivatives of phage lambda, e.g., NM989, λGT10 and λGT11, and other phages, e.g., M13 and filamentous single stranded phage DNA. A preferred vector is a bacterial artificial chromosome (BAC). A more preferred vector is pStreptoBAC, as described in Example 2.

In other embodiments, eukaryotic host cells, such as yeast, insect or mammalian cells, may be used. Yeast vectors include Yeast Integrating plasmids (e.g., YIp5) and Yeast Replicating plasmids (the YRp and YEp series plasmids), Yeast centromere plasmids (the YCp series plasmids), pGPD-2, 2μ plasmids and derivatives thereof, and improved shuttle vectors such as those described in Gietz and Sugino, Gene, 74, pp. 527-34 (1988) (YIplac, YEplac and YCplac). Expression in mammalian cells can be achieved using a variety of plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses). Useful vectors for insect cells include baculoviral vectors and pVL 941.

In addition, any of a wide variety of expression control sequences may be used in these vectors to express the DNA sequences of this invention. Such useful expression control sequences include the expression control sequences associated with structural genes of the foregoing expression vectors. Expression control sequences that control transcription include, e.g., promoters, enhancers and transcription termination sites. Expression control sequences in eukaryotic cells that control post-transcriptional events include splice donor and acceptor sites and sequences that modify the half-life of the transcribed RNA, e.g., sequences that direct poly(A) addition or binding sites for RNA-binding proteins. Expression control sequences that control translation include ribosome binding sites, sequences which direct targeted expression of the polypeptide to or within particular cellular compartments, and sequences in the 5′ and 3′ untranslated regions that modify the rate or efficiency of translation.

Examples of useful expression control sequences include, for example, the early and late promoters of SV40 or adenovirus, the lac system, the trp system, the TAC or TRC system, the T3 and T7 promoters, the major operator and promoter regions of phage lambda, the control regions of fd coat protein, the promoter for 3-phosphoglycerate kinase or other glycolytic enzymes, the promoters of acid phosphatase, e.g., Pho5, the promoters of the yeast α-mating system, the GALL or GAL10 promoters, and other constitutive and inducible promoter sequences known to control the expression of genes of prokaryotic or eukaryotic cells or their viruses, and various combinations thereof. Other expression control sequences include those from the daptomycin biosynthetic gene cluster, such as those described supra.

Preferred nucleic acid vectors also include a selectable or amplifiable marker gene and means for amplifying the copy number of the gene of interest. Such marker genes are well-known in the art. Nucleic acid vectors may also comprise stabilizing sequences (e.g., ori- or ARS-like sequences and telomere-like sequences), or may alternatively be designed to favor directed or non-directed integration into the host cell genome. Preferred marker genes and stabilizing sequences are disclosed in pStreptoBAC, which is described in Example 2. In a preferred embodiment, nucleic acid sequences of this invention are inserted in frame into an expression vector that allows high level expression of an RNA which encodes a protein comprising the encoded nucleic acid sequence of interest. Nucleic acid cloning and sequencing methods are well known to those of skill in the art and are described in an assortment of laboratory manuals, including Sambrook et al., supra, 1989; and Ausubel et al. Product information from manufacturers of biological, chemical and immunological reagents also provide useful information. Example 2 provides preferred nucleic acid cloning and sequencing methods.

Of course, not all vectors and expression control sequences will function equally well to express the nucleic acid sequences of this invention. Neither will all hosts function equally well with the same expression system. However, one of skill in the art may make a selection among these vectors, expression control sequences and hosts without undue experimentation and without departing from the scope of this invention. For example, in selecting a vector, the host must be considered because the vector must be replicated in it. The vector's copy number, the ability to control that copy number, the ability to control integration, if any, and the expression of any other proteins encoded by the vector, such as antibiotic or other selection markers, should also be considered.

In selecting an expression control sequence, a variety of factors should also be considered. These include, for example, the relative strength of the sequence, its controllability, and its compatibility with the nucleic acid sequence of this invention, particularly with regard to potential secondary structures. Unicellular hosts should be selected by consideration of their compatibility with the chosen vector, the toxicity of the product coded for by the nucleic acid sequences of this invention, their secretion characteristics, their ability to fold the polypeptide correctly, their fermentation or culture requirements, and the ease of purification from them of the products coded for by the nucleic acid sequences of this invention.

The recombinant nucleic acid molecules and more particularly, the expression vectors of this invention may be used to express the polypeptides of this invention as recombinant polypeptides in a heterologous host cell. The polypeptides of this invention may be full-length or less than full-length polypeptide fragments recombinantly expressed from the nucleic acid sequences according to this invention. Such polypeptides include analogs, derivatives and muteins that may or may not have biological activity. In a preferred embodiment, the polypeptides are expressed in a heterologous bacterial host cell. In a more preferred embodiment, the polypeptides are expressed in a heterologous Streptomyces host cell, still more preferably a S. lividans or S. fradiae host cell. See, e.g., Example 7, infra.

Transformation and other methods of introducing nucleic acids into a host cell (e.g., conjugation, protoplast transformation or fusion, transfection, electroporation, liposome delivery, membrane fusion techniques, high velocity DNA-coated pellets, viral infection and protoplast fusion) can be accomplished by a variety of methods which are well known in the art (see, for instance, Ausubel, supra, and Sambrook et al., supra). Bacterial, yeast, plant or mammalian cells are transformed or transfected with an expression vector, such as a plasmid, a cosmid, or the like, wherein the expression vector comprises the nucleic acid of interest. Alternatively, the cells may be infected by a viral expression vector comprising the nucleic acid of interest. Depending upon the host cell, vector, and method of transformation used, transient or stable expression of the polypeptide will be constitutive or inducible. One having ordinary skill in the art will be able to decide whether to express a polypeptide transiently or in a stable manner, and whether to express the protein constitutively or inducibly.

A wide variety of unicellular host cells are useful in expressing the DNA sequences of this invention. These hosts may include well known eukaryotic and prokaryotic hosts, such as strains of E. coli, Pseudomonas, Bacillus, Streptomyces, fungi, yeast, insect cells such as Spodoptera frugiperda (SF9), animal cells such as CHO, BHK, MDCK and various murine cells, e.g., 3T3 and WEHI cells, African green monkey cells such as COS 1, COS 7, BSC 1, BSC 40, and BMT 10, and human cells such as VERO, WI38, and HeLa cells, as well as plant cells in tissue culture. In a preferred embodiment, the host cell is Streptomyces. In a more preferred embodiment, the host cell is S. roseosporus, S. lividans or S. fradiae.

Particular details of the transfection, expression and purification of recombinant proteins are well documented and are understood by those of skill in the art. Further details on the various technical aspects of each of the steps used in recombinant production of foreign genes in bacterial cell expression systems can be found in a number of texts and laboratory manuals in the art. See, e.g., Ausubel et al., supra, and Sambrook et al., supra, and Kieser et al., supra, herein incorporated by reference.

Polypeptides

Thioesterases and Fragments Thereof

Another object of the invention is to provide a polypeptide derived from a thioesterase involved in daptomycin synthesis. In one embodiment, the polypeptide is derived from a daptomycin biosynthetic gene cluster. In a preferred embodiment, the polypeptide is derived from an integral or free thioesterase. In a more preferred embodiment, the polypeptide comprises the thioesterase domain of DptD or the amino acid sequence of DptH. In an even more preferred embodiment, the polypeptide comprises the amino acid sequence of the thioesterase domain of SEQ ID NO: 7 or the amino acid sequence of SEQ ID NO: 8. The polypeptide derived from a thioesterase may also be encoded by an S. roseosporus nucleic acid sequence from any one of BAC clones B12:01G05, B12:06A12, B12:12F06, B12:18H04, B12:20C09 or B12:03A05, preferably from B12:03A05. A polypeptide as defined herein may be produced recombinantly, as discussed supra, may be isolated from a cell that naturally expresses the protein, or may be chemically synthesized following the teachings of the specification and using methods well known to those having ordinary skill in the art. See, e.g., Examples 3-6.

The polypeptide may comprise a fragment of a thioesterase as defined herein. A polypeptide that comprises only a part or fragment of the entire thioesterase may or may not encode a polypeptide that has thioesterase activity. A polypeptide that does not have thioesterase activity, whether it is a fragment, analog, mutein, homologous protein or derivative, is nevertheless useful, especially for immunizing animals to prepare anti-thioesterase antibodies. However, in a preferred embodiment, the part or fragment encodes a polypeptide having thioesterase activity. Methods of determining whether a polypeptide has thioesterase activity are described infra. Further, in a preferred embodiment, the fragment comprises an amino acid sequence comprising the GXSXG thioesterase motif (see Example 3). In a more preferred embodiment, the fragment comprises an amino acid sequence comprising the thioesterase motif GWSFG or GTSLG, which are derived from the thioesterase domain of SEQ ID NO: 7 or the amino acid sequence of SEQ ID NO: 8, respectively.

One can produce fragments of a polypeptide encoding a thioesterase by truncating the DNA encoding the thioesterase and then expressing it recombinantly. Alternatively, one can produce a fragment by chemically synthesizing a portion of the full-length polypeptide. One may also produce a fragment by enzymatically cleaving either a recombinant polypeptide or an isolated naturally-occurring polypeptide. Methods of producing polypeptide fragments are well-known in the art (see, e.g., Sambrook et al. and Ausubel et al., supra). In one embodiment, a polypeptide comprising only a part or fragment of a thioesterase may be produced by chemical or enzymatic cleavage of a thioesterase. In a preferred embodiment, a polypeptide fragment is produced by expressing a nucleic acid molecule encoding a fragment of the thioesterase in a host cell.

Daptomycin NRPS Polypeptides, and Subunits and Fragments Thereof

Another object of the invention is to provide a polypeptide derived from a daptomycin NRPS or subunit thereof. The daptomycin NRPS comprises the subunits DptA, DptBC and DptD. As discussed in greater detail in Examples 3-6 below, each subunit comprises a number of modules that bind and activate specific building block substrates and to catalyze peptide chain formation and elongation. Further, each module comprises a number of domains that participate in condensation, adenylation and thiolation. In addition, some modules comprise a epimerization domain, discussed in greater detail in Example 6. DptD also comprises a thioesterase domain, as discussed supra and in Example 5.

In one embodiment, the polypeptide comprises an amino acid sequence from DptA, DptBC and/or DptD. In an even more preferred embodiment, the polypeptide comprises an amino acid sequence SEQ ID NOS: 9, 11 or 7. A daptomycin NRPS polypeptide may also be encoded by an S. roseosporus nucleic acid sequence from any one of BAC clones B12:01G05, B12:06A12, B12:12F06, B12:18H04, B12:20C09 or B12:03A05, preferably from B12:03A05. A polypeptide as defined herein may be produced recombinantly, as discussed supra, may be isolated from a cell that naturally expresses the protein, or may be chemically synthesized following the teachings of the specification and using methods well known to those having ordinary skill in the art. See, e.g., Examples 3-6 regarding amino acid sequences as well as modules and domains of DptA, DptBC and DptD.

The polypeptide may comprise a fragment of a daptomycin NRPS as defined herein. In one embodiment, a fragment comprises one or more complete modules of a daptomycin NRPS subunit. In another embodiment, a fragment comprises one or more domains of a daptomycin NRPS subunit. In yet another embodiment, a fragment may not comprise a complete domain or module but may comprise only a part of one or more domains or modules. A polypeptide that does not comprise a full domain or module of a daptomycin NRPS, whether it is a fragment, analog, mutein, homologous protein or derivative, is nevertheless useful, especially for immunizing animals to prepare anti-thioesterase antibodies. In a more preferred embodiment, the fragment comprises an amino acid sequence comprising at least that part of an adenylation domain that is required for binding to an amino acid. This part of the domain is delimited by the amino acid pocket code of a particular adenylation domain, as discussed below in Example 5.

As discussed above, one can produce fragments of a polypeptide of the invention recombinantly, by chemical synthesis or by enzymatic cleavage.

Polypeptides from S. roseosporus BAC Clones

Another object of the invention is to provide a polypeptide encoded by a nucleic acid molecule or part thereof from a S. roseosporus BAC clone of the invention. In one embodiment, the invention provides a polypeptide encoded by a nucleic acid molecule or part thereof from 1G05, B12:06A12, B12:12F06, B12:18H04, B12:20C09 or, preferably, B12:03A05. In a preferred embodiment, the invention provides a polypeptide comprising an amino acid sequence SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or encoded by a nucleic acid molecule comprising the nucleic acid sequence SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135. In another preferred embodiment, the invention provides a polypeptide that is DptE or DptF, a polypeptide having an amino acid sequence of SEQ ID NO: 15 or SEQ ID NO: 17, or encoded by dptE or dptF, or encoded by a nucleic acid sequence of SEQ ID NO: 16 or SEQ ID NO: 18. In another preferred embodiment, the invention provides an ABC transporter comprising an amino acid sequence SEQ ID NOS: 19, 21, 29, 45, 47, 49, 63, 67, 75 and 77, or encoded by a nucleic acid sequence of SEQ ID NOS: 20, 22, 30, 46, 48, 50, 64, 68, 76 or 78. In another preferred embodiment, the invention provides a polypeptide that is an oxidoreductase, such as a dehydrogenase; a transcriptional regulator involved in antibiotic resistance; NovABC-related polypeptides, which are involved in the biosynthesis of novobiocin, an antimicrobial agent; a monooxygenase; an acyl CoA thioesterase; a DNA helicase; or a DNA ligase, such as provided by a polypeptide having an amino acid sequence selected from SEQ ID NOS: 23, 25, 27, 29, 33, 35, 37, 91, 93, 97 and 99. In another preferred embodiment, the invention provides a polypeptide that is highly homologous to a Streptomyces polypeptide, such as provided by a polypeptide having an amino acid sequence selected from SEQ ID NOS: 61, 65, 69, 79, 81, 83, 85, 87, 95 and 101. A polypeptide as defined herein may be produced recombinantly, as discussed supra, may be isolated from a cell that naturally expresses the protein, or may be chemically synthesized following the teachings of the specification and using methods well known to those having ordinary skill in the art. The invention also provides a polypeptide that comprises a fragment of a nucleic acid molecule that encodes a polypeptide from a BAC clone, as defined herein. As discussed above, one can produce fragments of a polypeptide of the invention recombinantly, by chemical synthesis or by enzymatic cleavage.

Muteins, Homologous Proteins, Allelic Variants, Analogs and Derivatives

Another object of the invention is to provide polypeptides that are mutant proteins (muteins), fusion proteins, homologous proteins or allelic variants of the daptomycin NRPS, subunits thereof, thioesterases or the polypeptides encoded by the S. roseosporus BAC nucleic acid molecules or parts thereof provided herein. A mutant thioesterase may have the same or different enzymatic activity compared to a naturally-occurring thioesterase and comprises at least one amino acid insertion, duplication, deletion, rearrangement or substitution compared to the amino acid sequence of a native protein. In one embodiment, the mutein has the same or a decreased thioesterase activity compared to a naturally-occurring thioesterase. In another embodiment, the mutant thioesterase has an increased thioesterase activity compared to a naturally-occurring thioesterase. In a preferred embodiment, muteins of thioesterases of a daptomycin biosynthetic gene cluster may be used to alter thioesterase activity. See, e.g., Examples 12 and 13. In another embodiment, a mutant daptomycin NRPS or subunit thereof may have the same or different amino acid specificity, thiolation activity, condensation activity, or, if present, epimerization activity, as a naturally-occurring daptomycin NRPS. Daptomycin NRPS muteins may be used to alter amino acid recognition, binding, epimerization or other catalytic properties of an NRPS. See, e.g., Examples 12 and 16. Similarly, a mutein of a polypeptide encoded by the S. roseosporus BAC nucleic acid molecule of the invention may have a similar biological activity or a different one, but preferably has a similar biological activity.

A mutein of the invention may be produced by isolation from a naturally-occurring mutant microorganism or from a microorganism that has been experimentally mutagenized, may be produced by chemical manipulation of a polypeptide, or may be produced from a host cell comprising an altered nucleic acid molecule. In a preferred embodiment, the mutein is produced from a host cell comprising an altered nucleic acid molecule. Muteins may also be produced chemically by altering the amino acid residue to another amino acid residue using synthetic or semi-synthetic chemical techniques. One may produce muteins of a polypeptide by introducing mutations into the nucleic acid sequence encoding a daptomycin NRPS, subunit thereof or a thioesterase, or into a S. roseosporus BAC nucleic acid molecule, and then expressing it recombinantly. These mutations may be targeted, in which particular encoded amino acids are altered, or may be untargeted, in which random encoded amino acids within the polypeptide are altered. Muteins with random amino acid alterations can be screened for a particular biological activity, such as thioesterase activity, amino acid specificity, thiolation activity, epimerization activity, or condensation activity, as described below. Muteins may also be screened, e.g., for oxidoreductase activity, ABC transporter activity, monooxygenase activity, or DNA ligase or helicase activity using methods known in the art. Multiple random mutations can be introduced into the gene by methods well-known to the art, e.g., by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis and site-specific mutagenesis. Methods of producing muteins with targeted or random amino acid alterations are well known in the art. See, e.g., Sambrook et al., supra, Ausubel et al., supra, U.S. Pat. No. 5,223,408, and the references discussed supra, each herein incorporated by reference.

The invention also provides a polypeptide that is homologous to a daptomycin NRPS, subunit thereof, a thioesterase from a daptomycin biosynthetic gene cluster, or to a polypeptide encoded by a S. roseosporus BAC nucleic acid molecule as described herein. In one embodiment, the polypeptide is homologous to the thioesterase domain of DptD or to DptH, or to a polypeptide encoded by the thioesterase domain of dptD or by dptH. In a preferred embodiment, the polypeptide is homologous to a thioesterase having the amino acid sequence of the thioesterase domain of SEQ ID NO: 7 or having the amino acid sequence of SEQ ID NO: 8. In another embodiment, the polypeptide is homologous to DptA, DptBC or DptD, or to a polypeptide encoded by dptA, dptBC or dptD. In a more preferred embodiment, the polypeptide is homologous to a polypeptide having the amino acid sequence of SEQ ID NO: 9, 11 or 3. The invention also provides a polypeptide that is homologous to a polypeptide encoded by a nucleic acid molecule from a S. roseosporus BAC clone described herein, e.g., 1G05, B12:06A12, B12:12F06, B12:18H04, B12:20C09 or, preferably, B12:03A05. In a preferred embodiment, the invention provides a polypeptide homologous to a polypeptide comprising an amino acid sequence of SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or encoded by a nucleic acid molecule comprising a nucleic acid sequence selected from SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135.

In a preferred embodiment, the homologous polypeptide is one that exhibits significant sequence identity to a polypeptide of the invention. In a more preferred embodiment, the homologous polypeptide is one that exhibits at least 50%, 60%, 70%, or 80% sequence identity to a polypeptide comprising an amino acid sequence of SEQ ID NOS: 9, 11, 7 or 8 or SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136. In an even more preferred embodiment, the homologous polypeptide is one that exhibits at least 85%, 90%, 95%, 96%, 97%, 98% or 99% sequence identity to a polypeptide comprising an amino acid sequence of SEQ ID NOS: 9, 11, 7 or 8 or SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136.

The homologous protein may be a naturally-occurring one that is derived from another species, especially one derived from another Streptomyces species, or one derived from another Streptomyces roseosporus strain, wherein the homologous protein comprises an amino acid sequence that exhibits significant sequence identity to that of SEQ ID NOS: 9, 11, 7 or 8 or SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136. The naturally-occurring homologous protein may be isolated directly from the other species or strain. Alternatively, the nucleic acid molecule encoding the naturally-occurring homologous protein may be isolated and used to express the homologous protein recombinantly. In another embodiment, the homologous protein may be one that is experimentally produced by random mutation of a nucleic acid molecule and subsequent expression of the nucleic acid molecule. In another embodiment, the homologous protein may be one that is experimentally produced by directed mutation of one or more codons to alter the encoded amino acid of the polypeptide.

In another embodiment, the invention provides a polypeptide encoded by an allelic variant of a gene encoding a thioesterase from a daptomycin biosynthetic gene cluster, or a daptomycin NRPS or subunit thereof. In a preferred embodiment, the invention provides a polypeptide encoded by an allelic variant of dptA, dptBC, dptD or dptH. In an even more preferred embodiment, the polypeptide is encoded by an allelic variant of a gene that encodes a polypeptide having the amino acid sequence of SEQ ID NOS: 9, 11, 7 or 8. In a yet more preferred embodiment, the polypeptide is encoded by an allelic variant of a gene, wherein the gene has the nucleic acid sequence of SEQ ID NOS: 10, 12, 3 or 6. An allelic variant may have the same or different biological activity as the thioesterase, daptomycin NRPS or subunit thereof, described herein. In a preferred embodiment, an allelic variant is derived from another species of Streptomyces, even more preferably from a strain of Streptomyces roseosporus. In another embodiment, the invention provides a polypeptide encoded by an allelic variant of an S. roseosporus nucleic acid sequence from any one of BAC clones B12:01G05, B12:06A12, B12:12F06, B12:18H04, B12:20C09 or B12:03A05, preferably from B12:03A05. In a preferred embodiment, the polypeptide is encoded by an allelic variant of a gene that encodes a polypeptide having the amino acid sequence of SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136, or that is encoded by an allelic variant of a gene, wherein the gene has a nucleic acid sequence of SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135.

In another embodiment, the invention provides a derivative of a polypeptide of the invention. In a preferred embodiment, the derivative has been acetylated, carboxylated, phosphorylated, glycosylated or ubiquitinated. In another preferred embodiment, the derivative has been labeled with, e.g., radioactive isotopes such as ¹²⁵I, ³²P, ³⁵S, and ³H. In another preferred embodiment, the derivative has been labeled with fluorophores, chemiluminescent agents, enzymes, and antiligands that can serve as specific binding pair members for a labeled ligand. In a preferred embodiment, the polypeptide is a thioesterase involved in the biosynthesis of daptomycin. In an even more preferred embodiment, the polypeptide comprises the thioesterase domain of DptD or comprises the amino acid sequence of DptH, or is a thioesterase encoded by the thioesterase-encoding domain of dptD or by dptH. In another preferred embodiment, the polypeptide is a daptomycin NRPS or subunit thereof, more preferably DptA, DptBC or DptD, even more preferably a polypeptide encoded by dptA, dptBC or dptD. In a yet more preferred embodiment, the polypeptide has an amino acid sequence of SEQ ID NOS: 9, 11, 7 or 8 or is a mutein, allelic variant, homologous protein or fragment thereof. Preferably, a thioesterase derivative has a thioesterase activity that is the same or similar to a thioesterase involved in the biosynthesis of daptomycin, more preferably, the derivative has a thioesterase activity that is the same or similar to a thioesterase having an amino acid sequence of the thioesterase domain of SEQ ID NO: 7 or having the amino acid sequence of SEQ ID NO: 8. In another preferred embodiment, a daptomycin NRPS or NRPS subunit derivative has the same or similar activity as a naturally-occurring daptomycin NRPS or subunit thereof. In yet another embodiment, the derivative is derived from a polypeptide encoded by a nucleic acid molecule from a S. roseosporus nucleic acid sequence from any one of BAC clones B12:01G05, B12:06A12, B12:12F06, B12:18H04, B12:20C09 or, preferably, B12:03A05. In a preferred embodiment, the derivative is derived from a polypeptide having an amino acid sequence of SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136, or that is encoded by a gene having a nucleic acid sequence of SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135.

The invention also provides non-peptide analogs. In a preferred embodiment, the non-peptide analog is structurally similar to a thioesterase involved in daptomycin synthesis, to a daptomycin NRPS or subunit thereof, or to a polypeptide encoded by a nucleic acid molecule from an S. roseosporus BAC clone, but in which one or more peptide linkages is replaced by a linkage selected from the group consisting of —CH₂NH—, —CH₂S—, —CH₂—CH₂—, —CH═CH— (cis and trans), —COCH₂—, —CH(OH)CH₂— and —CH₂SO—. In another embodiment, the non-peptide analog comprises substitution of one or more amino acids of a thioesterase or daptomycin NRPS or subunit thereof with a D-amino acid of the same type in order to generate more stable peptides. Preferably, both a non-peptide and a peptide analog has a biological activity that is the same or similar to the naturally-occurring polypeptide involved in the biosynthesis of daptomycin, more preferably, the analog has a biological activity that is the same or similar to the polypeptide having an amino acid sequence of SEQ ID NOS: 9, 11, 7 or 8. The invention also provides analogs of polypeptides encoded by an S. roseosporus nucleic acid sequence from any one of BAC clones B12:01G05, B12:06A12, B12:12F06, B12:18H04, B12:20C09 or B12:03A05, preferably from B12:03A05. The invention provides an analog of a polypeptide having an amino acid sequence of SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136, or that is encoded by a gene having a nucleic acid sequence of SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135.

Fusion Proteins

The polypeptides of this invention may be fused to other molecules, such as genetic, enzymatic or chemical or immunological markers such as epitope tags. Fusion partners include, inter alia, myc, hemagglutinin (HA), GST, immunoglobulins, β-galactosidase, biotin trpE, protein A, β-lactamase, α-amylase, maltose binding protein, alcohol dehydrogenase, polyhistidine (for example, six histidine at the amino and/or carboxyl terminus of the polypeptide), lacZ, green fluorescent protein (GFP), yeast α mating factor, GAL4 transcription activation or DNA binding domain, luciferase, and serum proteins such as ovalbumin, albumin and the constant domain of IgG. See, e.g., Godowski et al., 1988, and Ausubel et al., supra. Fusion proteins may also contain sites for specific enzymatic cleavage, such as a site that is recognized by enzymes such as Factor XIII, trypsin, pepsin, or any other enzyme known in the art. Fusion proteins will typically be made by either recombinant nucleic acid methods, as described above, chemically synthesized using techniques such as those described in Merrifield, 1963, herein incorporated by reference, or produced by chemical cross-linking.

Tagged fusion proteins permit easy localization, screening and specific binding via the epitope or enzyme tag. See Ausubel, 1991, Chapter 16. Some tags allow the protein of interest to be displayed on the surface of a phagemid, such as M13, which is useful for panning agents that may bind to the desired protein targets. Another advantage of fusion proteins is that an epitope or enzyme tag can simplify purification. These fusion proteins may be purified, often in a single step, by affinity chromatography. For example, a His⁶ tagged protein can be purified on a Ni affinity column and a GST fusion protein can be purified on a glutathione affinity column. Similarly, a fusion protein comprising the Fc domain of IgG can be purified on a Protein A or Protein G column and a fusion protein comprising an epitope tag such as myc can be purified using an immunoaffinity column containing an anti-c-myc antibody. It is preferable that the epitope tag be separated from the protein encoded by the nucleic acid molecule of the invention by an enzymatic cleavage site that can be cleaved after purification.

A second advantage of fusion proteins is that the epitope tag can be used to bind the fusion protein to a plate or column through an affinity linkage for screening targets.

Therefore, in another aspect, the invention provides a fusion protein comprising all or a part of a thioesterase derived from a daptomycin biosynthetic gene cluster and provides a nucleic acid molecule that encodes such a fusion protein. Another aspect provides a fusion protein comprising all or a part of a daptomycin NRPS or subunit thereof and provides a nucleic acid molecule encoding such a protein. See, e.g., Examples 11-16. The invention also provides a fusion protein comprising all or part of a polypeptide encoded by a nucleic acid molecule from any one of BAC clones B12:01G05, B12:06A12, B12:12F06, B12:18H04, B12:20C09 or B12:03A05. In a preferred embodiment, the fusion protein comprises all or a part of a polypeptide encoded by one or more of dptA, dptBC, dptD or dptH. In another preferred embodiment, the fusion protein comprises a polypeptide encoded by a nucleic acid molecule that selectively hybridizes to dptA, dptBC, dptD or dptH. In a more preferred embodiment, the fusion protein comprises a polypeptide having an amino acid sequence of SEQ ID NOS: 9, 11, 7 or 8, or comprises a polypeptide that is a fragment, mutein, homologous protein, derivative or analog thereof. In an even more preferred embodiment, the nucleic acid molecule encoding the fusion protein comprises all or part of the nucleic acid sequence of SEQ ID NOS: 10, 12, 3 or 6, or comprises all or part of a nucleic acid sequence that selectively hybridizes or is homologous to a nucleic acid molecule comprising said nucleic acid sequence. The invention also provides fusion proteins comprising polypeptide sequences encoded by an S. roseosporus nucleic acid sequence from any one of BAC clones B12:01G05, B12:06A12, B12:12F06, B12:18H04, B12:20C09 or B12:03A05, preferably from B12:03A05. The invention provides a fusion protein comprising a polypeptide having an amino acid sequence of SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136, or comprising a polypeptide that is a fragment, mutein, homologous protein, derivative or analog thereof. The invention also provides a fusion protein comprising a polypeptide encoded by SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135, or comprising all or part of a nucleic acid sequence that selectively hybridizes or is homologous to a nucleic acid molecule comprising said nucleic acid sequence.

In one aspect of the invention, the fusion protein that comprises all or a part of a thioesterase derived from a daptomycin biosynthetic gene cluster comprises other modules (including heterologous or hybrid modules) from a polypeptide involved in non-ribosomal protein synthesis. See, e.g., Examples 12E, G and H and Example 13. In another preferred embodiment, the fusion protein comprises one or more amino acid sequences that encode thioesterases, wherein the thioesterases may be identical to one another or may be different. See, e.g., Examples 11E-G (duplication of daptomycin thioesterase genes), Example 12 (producing modified NRPS thioesterase fusion proteins) and Example 13 (producing free thioesterase fusion proteins).

In another embodiment, the invention provides a fusion protein that is a hybrid of amino acid sequences from two or more different thioesterases and a nucleic acid molecule that encodes such a fusion protein. The hybrid fusion protein may consist of two, three or more portions of different thioesterases. The hybrid thioesterase may have a different or the same specificity.

Methods to Assay Thioesterase and Daptomycin NRPS Activity

There are a number of methods known in the art to determine whether a fragment, mutein, homologous protein, analog, derivative or fusion protein of a thioesterase has the same, enhanced or decreased biological activity as a wild-type thioesterase polypeptide. In one embodiment, a thioesterase assay which monitors cleavage of a suitable thioester bond and/or release of a corresponding product is performed in vitro. Any of a number of thioesterase assays well-known in the art may be used, including those which use photo- or radio-labeled substrates.

In a preferred embodiment, thioesterase activity associated with peptide synthesis by a NRPS is determined using cellular assays. For example, a nucleic acid molecule encoding a fragment, mutein, homologous protein or fusion protein may be introduced into a bacterial cell comprising a daptomycin biosynthetic gene cluster absent one or both of the thioesterase domains of dptD or dptH. Alternatively, the nucleic acid molecule may be introduced into a bacterial cell comprising a different biosynthetic gene cluster that produces a different compound, e.g., a different lipopeptide. In a preferred embodiment, the bacterial cell may be S. lividans. The nucleic acid molecule may be introduced into the bacterial cell by any method known in the art, including conjugation, transformation, electroporation, protoplast fusion or the like. The bacterial cell comprising the nucleic acid molecule is incubated under conditions in which the polypeptide encoded by the nucleic acid molecule is expressed. After incubation, the bacterial cells may be analyzed by, e.g., HPLC and/or LC/MS, to determine if the bacterial cells produce the desired lipopeptide. See, e.g., the method of expressing daptomycin described in Examples 7-9, infra. When the thioesterase activity is associated with synthesis of a peptide having an anti-cell growth property (e.g., an antibiotic, antifungal, antiviral or antimitotic agent) a desired assay known to those of skill in the art may be used.

Alternatively, a fragment, mutein, homologous protein, analog, derivative or fusion protein of a thioesterase may be introduced into a cell, particularly a bacterial cell, comprising a daptomycin biosynthetic gene cluster absent one or both of the thioesterase domain of dptD or dptH. After incubation, the bacterial cells may be analyzed by, e.g., HPLC and/or LC/MS, as described in Example 7, to determine if the bacterial cells produce the desired lipopeptide. The same method can be used with a cell comprising a different biosynthetic gene cluster that produces a different compound, e.g., a different lipopeptide.

In a preferred embodiment, a fragment, mutein, homologous protein, analog, derivative or fusion protein comprises an amino acid sequence comprising the GXSXG thioesterase motif (see Example 3). In a more preferred embodiment, a fragment, mutein, homologous protein, analog or derivative comprises an amino acid sequence comprising the thioesterase motif GWSFG (SEQ ID NO: 166) or GTSLG (SEQ ID NO: 167), which are derived from SEQ ID NO: 7 and SEQ ID NO: 8, respectively.

Similar methods known in the art may be used to determine whether a fragment, mutein, homologous protein, analog, derivative or fusion protein of a daptomycin NRPS or subunit thereof has the same or different biological activity as a wild-type NRPS or subunit thereof.

Antibodies

The polypeptides encoded by the genes of this invention may be used to elicit polyclonal or monoclonal antibodies that bind to a polypeptide of this invention, as well as a fragment, mutein, homologous protein, analog, derivative or fusion protein thereof, using a variety of techniques well known to those of skill in the art. Antibodies directed against the polypeptides of this invention are immunoglobulin molecules or portions thereof that are immunologically reactive with the polypeptide of the present invention.

Antibodies directed against a polypeptide of the invention may be generated by immunization of a mammalian host. Such antibodies may be polyclonal or monoclonal. Preferably they are monoclonal. Methods to produce polyclonal and monoclonal antibodies are well known to those of skill in the art. For a review of such methods, see Harlow and Lane, Antibodies: A Laboratory Manual (1988) and Ausubel et al. supra, herein incorporated by reference. Determination of immunoreactivity with a polypeptide of the invention may be made by any of several methods well known in the art, including by immunoblot assay and ELISA.

Monoclonal antibodies with affinities of 10⁻⁸ M⁻¹ or preferably 10⁻⁹ to 10⁻¹⁰ M⁻¹ or stronger are typically made by standard procedures as described, e.g., in Harlow and Lane, 1988. Briefly, appropriate animals are selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to the antigenic polypeptides, or alternatively, to selection of libraries of antibodies in phage or similar vectors. See Huse et al., 1989. The polypeptides and antibodies of the present invention may be used with or without modification. Frequently, polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241, herein incorporated by reference. Also, recombinant immunoglobulins may be produced (see U.S. Pat. No. 4,816,567, herein incorporated by reference).

An antibody of this invention may also be a hybrid molecule formed from immunoglobulin sequences from different species (e.g., mouse and human) or from portions of immunoglobulin light and heavy chain sequences from the same species. An antibody may be a single-chain antibody or a humanized antibody. It may be a molecule that has multiple binding specificities, such as a bifunctional antibody prepared by any one of a number of techniques known to those of skill in the art including the production of hybrid hybridomas, disulfide exchange, chemical cross-linking, addition of peptide linkers between two monoclonal antibodies, the introduction of two sets of immunoglobulin heavy and light chains into a particular cell line, and so forth.

The antibodies of this invention may also be human monoclonal antibodies, for example those produced by immortalized human cells, by SCID-hu mice or other non-human animals capable of producing “human” antibodies, or by the expression of cloned human immunoglobulin genes. The preparation of humanized antibodies is taught by U.S. Pat. Nos. 5,777,085 and 5,789,554, herein incorporated by reference.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

In a preferred embodiment, an antibody of the present invention binds to a thioesterase involved in daptomycin synthesis or to a daptomycin NRPS or subunit thereof. In a more preferred embodiment, the antibody binds to a polypeptide encoded by dptA, dptBC, dptD or dptH, or to a fragment thereof. In another preferred embodiment, the antibody binds to a polypeptide encoded by a nucleic acid molecule that selectively hybridizes to dptA, dptBC, dptD or dptH. In a more preferred embodiment, the antibody binds to a polypeptide having an amino acid sequence of SEQ ID NOS: 9, 11, 7 or 8, or binds to a polypeptide that is fragment, mutein, homologous protein, derivative, analog or fusion protein thereof. In an even more preferred embodiment, the antibody binds to a polypeptide encoded by a nucleic acid molecule comprising all or part of the nucleic acid sequence of SEQ ID NOS: 10, 12, 3 or 6. In another embodiment, the antibody binds to a polypeptide encoded by a nucleic acid molecule that comprises all or part of a nucleic acid sequence that selectively hybridizes or is homologous to a nucleic acid molecule comprising a nucleic acid sequence of SEQ ID NOS: 10, 12, 3 or 6.

The invention provides an antibody that selectively binds to a polypeptide encoded by an S. roseosporus nucleic acid sequence from any one of BAC clones B12:01G05, B12:06A12, B12:12F06, B12:18H04, B12:20C09 or B12:03A05, preferably from B12:03A05. The polypeptide may comprise an amino acid sequence selected from SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136 or is encoded by a nucleic acid sequence SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135. Preferably, the antibody selectively binds to a polypeptide comprising an amino acid sequence selected from SEQ ID NOS: 23, 25, 27, 29, 33, 35, 37, 91, 93, 97, 99, 110 or 112 or from SEQ ID NOS: 61, 65, 69, 79, 81, 83, 85, 87, 95 and 101. The invention also provides an antibody that selectively binds to a fragment, mutein, homologous protein, derivative, analog or fusion protein thereof.

Computer Readable Means

A further aspect of the invention is a computer readable means for storing the nucleic acid and amino acid sequences of the instant invention. In a preferred embodiment, the invention provides a computer readable means for storing all of the nucleic acid and amino acid sequences described herein, as the complete set of sequences or in any combination. The records of the computer readable means can be accessed for reading and display and for interface with a computer system for the application of programs allowing for the location of data upon a query for data meeting certain criteria, the comparison of sequences, the alignment or ordering of sequences meeting a set of criteria, and the like.

The nucleic acid and amino acid sequences of the invention are particularly useful as components in databases useful for search analyses as well as in sequence analysis algorithms. As used herein, the terms “nucleic acid sequences of the invention” and “amino acid sequences of the invention” mean any detectable chemical or physical characteristic of a polynucleotide or polypeptide of the invention that is or may be reduced to or stored in a computer readable form. These include, without limitation, chromatographic scan data or peak data, photographic data or scan data therefrom, and mass spectrographic data.

This invention provides computer readable media having stored thereon sequences of the invention. A computer readable medium may comprise one or more of the following: a nucleic acid sequence comprising a sequence of a nucleic acid sequence of the invention; an amino acid sequence comprising an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of one or more nucleic acid sequences of the invention; a data set representing a nucleic acid sequence encoding an amino acid sequence comprising the sequence of an amino acid sequence of the invention; a set of nucleic acid sequences wherein at least one of said sequences comprises the sequence of a nucleic acid sequence of the invention; a set of amino acid sequences wherein at least one of said sequences comprises the sequence of an amino acid sequence of the invention; a data set representing a nucleic acid sequence comprising the sequence of a nucleic acid sequence of the invention; a data set representing a nucleic acid sequence encoding an amino acid sequence comprising the sequence of an amino acid sequence of the invention. The computer readable medium can be any composition of matter used to store information or data, including, for example, commercially available floppy disks, tapes, hard drives, compact disks, and video disks.

Also provided by the invention are methods for the analysis of character sequences, particularly genetic sequences. Preferred methods of sequence analysis include, for example, methods of sequence homology analysis, such as identity and similarity analysis, RNA structure analysis, sequence assembly, cladistic analysis, sequence motif analysis, open reading frame determination, nucleic acid base calling, and sequencing chromatogram peak analysis.

A computer-based method is provided for performing nucleic acid homology identification. This method comprises the steps of providing a nucleic acid sequence comprising the sequence a nucleic acid of the invention in a computer readable medium; and comparing said nucleic acid sequence to at least one nucleic acid or amino acid sequence to identify homology.

A computer-based method is also provided for performing amino acid homology identification, said method comprising the steps of: providing an amino acid sequence comprising the sequence of an amino acid of the invention in a computer readable medium; and comparing said an amino acid sequence to at least one nucleic acid or an amino acid sequence to identify homology.

A computer based method is still further provided for assembly of overlapping nucleic acid sequences into a single nucleic acid sequence, said method comprising the steps of: providing a first nucleic acid sequence comprising the sequence of a nucleic acid of the invention in a computer readable medium; and screening for at least one overlapping region between said first nucleic acid sequence and a second nucleic acid sequence.

Methods of Using Nucleic Acid Molecules as Probes and Primers

In one embodiment, a nucleic acid molecule of the invention may be used as a probe or primer to identify or amplify a nucleic acid molecule that selectively hybridizes to the nucleic acid molecule. In a preferred embodiment, the probe or primer is derived from a nucleic acid molecule encoding a daptomycin NRPS, subunit thereof or thioesterase from a daptomycin biosynthetic gene cluster. The probe or primer may also be derived from an expression control sequence derived from a daptomycin NRPS or thioesterase gene of a daptomycin biosynthetic gene cluster. In a preferred embodiment, the probe or primer is derived from dptA, dptBC, dptD or dptH. In a more preferred embodiment, the probe or primer is derived from a nucleic acid molecule that encodes a polypeptide having an amino acid sequence of SEQ ID NOS: 9, 11, 7 or 8. In a yet more preferred embodiment, the probe or primer is derived from a nucleic acid molecule that has a nucleic acid sequence of SEQ ID NOS: 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 105, 107, 109, 111, 113, 115, 117, 119, 121, 123, 125, 127, 129, 131, 133 or 135. In another embodiment, the probe or primer is derived from a nucleic acid sequence that encodes SEQ ID NOS: 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 104, 108, 110, 112, 114, 116, 118, 120, 122, 124, 126, 128, 130, 132, 134 or 136.

In general, a probe or primer is at least 10 nucleotides in length, more preferably at least 12, more preferably at least 14 and even more preferably at least 16 nucleotides in length. In an even more preferred embodiment, the probe or primer is at least 18 nucleotides in length, even more preferably at least 20 nucleotides and even more preferably at least 22 nucleotides in length. Primers and probes may also be longer in length. For instance, a probe or primer may be 25 nucleotides in length, or may be 30, 40 or 50 nucleotides in length. Methods of performing nucleic acid hybridization using oligonucleotide probes are well-known in the art. See, e.g., Sambrook et al., supra. See, e.g., Chapter 11 and pages 11.31-11.32 and 11.40-11.44, which describes radiolabeling of short probes, and pages 11.45-11.53, which describes hybridization conditions for oligonucleotide probes, including specific conditions for probe hybridization (pages 11.50-11.51). Methods of performing PCR using primers are also well-known in the art. See, e.g., Sambrook et al., supra and Ausubel et al., supra. PCR methods may be used to identify and/or isolate allelic variants and fragments of the nucleic acid molecules of the invention; PCR may also be used to identify and/or isolate nucleic acid molecules that hybridize to the primers and that may be amplified, and may be used to isolate nucleic acid molecules that encode homologous proteins, analogs, fusion protein or muteins of the invention.

Methods of Using Thioesterases for Biosynthesis of Compounds—Manipulations of Dpt Genes

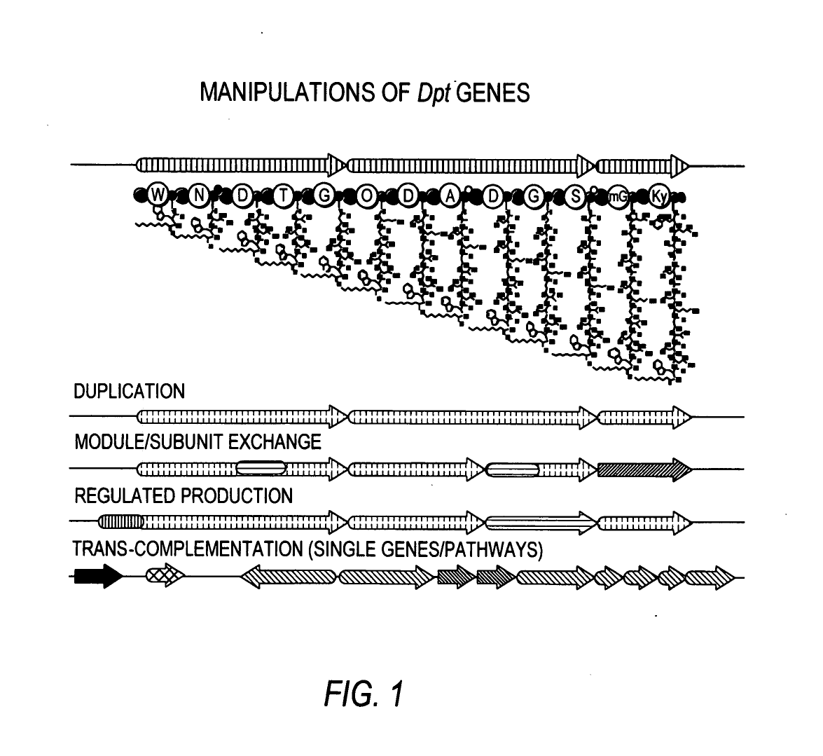

Genes of the daptomycin biosynthetic gene cluster of the invention may be manipulated in a variety of ways to produce new biosynthetic peptide products or to alter the regulation of one or more genes expressed from the gene cluster. See, e.g., FIG. 1.

Disruption of a Gene Encoding a Thioesterase

In one aspect, the invention provides a method of disrupting or deleting a gene encoding a thioesterase that is involved in a NRPS or PKS pathway in a bacterial cell. Preferably, the method comprises the step of disrupting or deleting a gene or portion thereof that encodes a thioesterase in a daptomycin biosynthetic gene cluster. Disruption or deletion of a gene encoding an integral thioesterase would be likely to result in the production of compounds that are intermediates to the final product. In one aspect, a gene or portion thereof encoding an integral thioesterase may be disrupted or deleted. In a preferred embodiment, disruption or deletion of a gene encoding an integral thioesterase of the daptomycin biosynthetic gene cluster in S. roseosporus would produce a linear lipopeptide compound. The linear lipopeptide compound may be used directly if its release from the NRPS were to be catalyzed by a different endogenous or exogenously provided thioesterase activity within the host cell. Such linear lipopeptide compounds, if not released from the NRPS by an endogenous thioesterase activity, may be useful intermediates for testing potential but as yet unidentified thioesterase polypeptides or for testing thioesterase fusion, fragment, mutein, derivative, analog or homolog polypeptides for activity. The linear lipopeptide compound may alternatively be used as an intermediate for production of novel lipopeptides.

In another aspect, a gene encoding a free thioesterase may be disrupted or deleted in a bacterial cell comprising an NRPS. Because free thioesterases are thought to be involved in proofreading of the peptide compounds produced in NRPS, disruption or deletion of a gene encoding a free thioesterase may lead to the production of compounds that have mutations compared to the compound produced in the presence of the free thioesterase. These mutated compounds may be used to generate novel lipopeptides. See, e.g., Example 13.

In a preferred embodiment, the method comprises the step of disrupting or deleting the thioesterase-encoding portion of dptD or disrupting or deleting dptH in a daptomycin biosynthetic gene cluster. In an even more preferred embodiment, the method comprises the step of disrupting or deleting a gene encoding a thioesterase having an amino acid sequence of the thioesterase domain of SEQ ID NO: 7 or having the amino acid sequence of SEQ ID NO: 8. The invention also comprises a method of disrupting or deleting a gene encoding a thioesterase wherein the gene is one that selectively hybridizes or is homologous to a gene encoding a thioesterase having an amino acid sequence of the thioesterase domain of SEQ ID NO: 7 or the amino acid sequence of SEQ ID NO: 8. In another preferred embodiment, disruption or deletion of a thioesterase may be combined with the methods of altering the gene cluster involved in non-ribosomal peptide synthesis, as described below.

Disruption of a gene encoding a thioesterase may be accomplished by any method known to one having ordinary skill in the art following the teachings of the instant specification. In a preferred embodiment, disruption of a gene encoding a thioesterase may be accomplished by targeted gene disruption using methods taught, e.g., in Hosted and Baltz, J. Bacteriol., 179, pp. 180-186 (1997); Butler et al., Chem. Biol., 6, pp. 287-292 (1999); and Xue et al., Proc. Natl. Acad. Sci. U.S.A., 95, pp. 12111-12116 (1998), each of which is incorporated herein by reference in its entirety. See, e.g., Example 11.

Alteration of Site of Cyclization and Cyclic Peptide Produced Using Thioesterases

In a naturally-occurring polypeptide involved in NRPS, an integral thioesterase is located at the carboxy-terminus of the polypeptide, where it is involved in product cyclization. In one aspect, the invention provides a method to alter the site of cyclization of a cyclic peptide (or release of a linear peptide) by changing the location of a module encoding a thioesterase. In one embodiment, the site of cyclization may be altered by inserting the module encoding the thioesterase into the gene encoding the polypeptide involved in NRPS in a region that is upstream of the region in which the thioesterase module naturally occurs. In this embodiment, the cyclic peptide that is produced will be smaller than the naturally-occurring cyclic peptide. See, e.g., Example 12.

In a preferred embodiment, the module encodes an integral thioesterase from a daptomycin biosynthetic gene cluster. In a more preferred embodiment, the module comprises the thioesterase domain of DptD. In an even more preferred embodiment, the module encodes a polypeptide having all or a portion of the amino acid sequence of SEQ ID NO: 7, preferably a portion of SEQ ID NO: 7 that comprises the thioesterase domain. In another preferred embodiment, the module comprises a nucleic acid molecule that is homologous to or selectively hybridizes to a nucleic acid molecule encoding all or a portion of the thioesterase domain of SEQ ID NO: 7 or to a nucleic acid molecule encoding the thioesterase domain that comprises all or a portion of the nucleic acid sequence of SEQ ID NO: 3.

Alternatively, other modules that are involved in adding amino acids to the peptide (or otherwise modifying amino acids within the peptide) may be inserted upstream of the module encoding the thioesterase. See, e.g., Example 12. Such modules include a minimal module comprising at least an adenylation domain and a thiolation or acyl carrier domain. In a preferred embodiment, the inserted module would also include a condensation domain. Additional domains may also be inserted upstream of the thioesterase module including an M domain, an E domain and/or a Cy domain. The type of module(s) that would be inserted upstream of the thioesterase domain would depend upon the type of amino acid residues that were desired. Methods of inserting modules that will add and/or modify a specific amino acid are well known in the art. See, e.g., Mootz et al., Curr. Opin. Biotechnol., 10, pp. 341-348 (1999), herein incorporated by reference in its entirety. Addition of one or more modules upstream of the thioesterase will produce a polypeptide involved in NRPS that is capable of synthesizing a cyclic peptide that is larger and that may contain different amino acid residues than the naturally-occurring cyclic peptide.

In Vivo Use of Thioesterases

Another use of the genes of the present invention is to improve the yield of a product in a cell expressing an NRPS. See, e.g., Example 11. Nucleic acid molecules that may be used to increase yield include nucleic acid molecules that encode positive regulatory factors, acyl CoA thioesterase, ABC transporters, NovABC-related polypeptides, DptA, DptBC, DptD, polypeptides that encode daptomycin resistance and daptomycin thioesterases, including DptD and DptH. The complete daptomycin biosynthetic gene cluster, daptomycin NRPS or any domain or subunit thereof may also be duplicated. In a preferred embodiment, a free and/or an integral thioesterase from a daptomycin biosynthetic gene cluster are introduced into a cell to improve production of daptomycin. In another preferred embodiment, the additional copies of a thioesterase may be introduced into a cell comprising altered NRPS polypeptides, as described supra. Without wishing to be bound by any theory, additional copies of a free and/or an integral thioesterase may improve the NRPS processing of the peptide by increasing the proofreading capacity (e.g., the free thioesterase) or the cyclization and/or peptide release capacity (e.g., the integral thioesterase) of the bacterial cell.

In a preferred embodiment, additional copies of a nucleic acid molecule encoding thioesterase may be introduced into a cell. See, e.g., Example 11. Introduction of the thioesterase may be performed by any method known in the art. In a more preferred embodiment, the additional copies of the gene are under the regulatory control of strong expression control sequences. These sequences may be derived from another thioesterase gene or may be derived from heterologous sequences, as described supra. Further, a nucleic acid molecule encoding a thioesterase may be introduced into a cell such that it is expressed as a separate polypeptide. This may be especially useful for a free thioesterase. Alternatively, a nucleic acid molecule encoding a thioesterase may be introduced into a cell such that it forms part of a multi-domain protein. This can be accomplished, e.g., by homologous recombination into a polypeptide which forms or interacts with an NRPS. This may be especially useful, although not required, for an integral thioesterase.

In another embodiment, copies of a free and/or an integral thioesterase may be introduced into a cell that expresses a NRPS complex that is other than a daptomycin biosynthetic gene cluster. See, e.g., Example 13. In one preferred embodiment, the complex is a NRPS complex. In another preferred embodiment, the complex is a PKS complex or a mixed PKS/NRPS complex. Numerous PKS and NRPS complexes are known in the art. See, e.g., complexes that produce vancomycin, bleomycin, A54145, CDA, amphomycin, echinocandin, cyclosporin, erythromycin, tylosin, monensin, avermectin, penicillin, cephalosporin, pristinamycins, erythromycin, rapamycin, spinosyn, didemnin, discobahamian, and epothilone. As described above, addition of a free and/or an integral thioesterase may improve the NRPS or PKS processing of a peptide by increasing the proofreading capacity (the free thioesterase) or the cyclization capacity (the integral thioesterase) of the bacterial cell. Addition of a free and/or integral thioesterase may be achieved by the methods described above.

In a preferred embodiment, a nucleic acid molecule encoding a thioesterase that is introduced into a cell is a thioesterase from a daptomycin biosynthetic gene cluster. In a preferred embodiment, the gene is the thioesterase-encoding domain of dptD or is dptH. More preferably, the nucleic acid molecule encodes a thioesterase having an amino acid sequence of the thioesterase domain of SEQ ID NO: 7 or SEQ ID NO: 8, or is a homologous protein, fusion protein, mutein, derivative, analog or fragment thereof having thioesterase activity.

Methods of Altering Gene Clusters for Production of Novel Compounds by NRPS

Alteration of NRPS Polypeptide Modules and Domains

In another aspect, the invention provides a method of altering the number or position of the modules in an NRPS. In one embodiment, one or more modules may be deleted from the NRPS. These deletions will result in synthesis by the NRPS of a peptide product that is shorter than the naturally-occurring one. In another embodiment, one or more domains may be deleted from the NRPS. In this case, the product produced by the NRPS will have a chemical change relative to the peptide produced in the absence of the deletion, e.g., if an epimerization and/or methylation domain is deleted.

In another embodiment, one or more modules or domains may be added to the NRPS. In this case, the peptide synthesized by the NRPS will be longer than the naturally-occurring one or will have an additional chemical change, respectively. For instance, if an epimerization domain or a methylation domain is added, the resultant peptide will contain an extra D-amino acid or will contain a methylated amino acid, respectively. In a yet further embodiment, one or more modules may be mutated, e.g., an adenylation domain may be mutated such that it has a different amino acid specificity than the naturally-occurring adenylation domain. The amino acid pocket code for the daptomycin NRPS—which determines which amino acid will bind within each adenylation domain of modules 1-13—is described in Example 5; see also Table 2. With the amino acid code in hand, one of skill in the art can perform mutagenesis, by a variety of well known techniques, to exchange the code in one module for another code, thus altering the ultimate amino acid composition and/or sequence of the resulting peptide synthesized by the altered NRPS. See, e.g., Example 12A. In another embodiment, one or more subunits may be added or deleted to the NRPS.

In a still further embodiment, one or more domains, modules or subunits may be substituted with another domain, module or subunit in order to produce novel peptides by complementation. In this case, the peptide produced by the altered NRPS will have, e.g., one or more different amino acids compared to the naturally-occurring peptide. In addition, different combinations of insertions, deletions, substitutions and mutations of domains, modules or subunits may be used to produce a peptide of interest. For instance, one may substitute a modified module, domain or subunit for a naturally-occurring one, or may substitute a naturally-occurring module, domain or subunit from the NRPS from one organism for a module, domain or subunit of an NRPS from another organism. See, e.g., Example 12C. Modifications of the modules, domains and subunits may be performed by site-directed mutagenesis, domain exchange (for module or subunit modification), deletion, insertion or substitution of a domain in a module or subunit, or deletion, insertion or substitution of a module in a subunit. Further, a domain, module or subunit may be disrupted such that it does not function using any method known in the art. These disruptions include, e.g., such techniques as a single crossover disruptant or replacement through homologous recombination by another gene (e.g., a gene that permits selection or screening).

The products produced by the modified NRPS complexes will have different incorporated amino acids, different chemical alterations of the amino acids (e.g., methylation and epimerization) and may be shorter or longer than the native lipopeptides. The domains, modules or subunits may be derived from any number of NRPS desired, including two, three or four NRPS. Further, the invention contemplates these altered NRPS complexes with and without an integral thioesterase domain. See, e.g., Example 12B-J.

The source of the modules, domains and/or subunits may be derived from the daptomycin biosynthetic gene cluster NRPS or may be derived from the NRPS that encodes another lipopeptide or other peptide source. These peptide sources include glycopeptide gene clusters, mixed pathway gene clusters and siderophore gene clusters. Further, the source of the modules, domains and/or subunits may be obtained from any appropriate source, including both streptomycete and non-streptomycete sources. Non-streptomycete sources include actinomycetes, e.g., Amycolatopsis; prokaryotic non-actinomycetes, e.g., Bacillus and cyanobacteria; and non-bacterial sources, e.g., fungi.

An NRPS or portion thereof may be heterologous to a host cell of interest or may be endogenous to the host cell. In one embodiment, the daptomycin NRPS or a portion thereof (e.g., a domain, module or subunit thereof) is introduced into the host cell on any vector known to one having ordinary skill in the art, e.g., a plasmid, a cosmid, bacteriophage or BAC. The host cell into which the daptomycin NRPS or portion thereof is introduced may contain an endogenous NRPS or portion thereof (e.g., a domain, module or subunit thereof). Alternatively, a heterologous NRPS or portion thereof may be introduced into the host cell containing the heterologous daptomycin NRPS or portion thereof. The daptomycin NRPS, other NRPS, or domain, module or subunit of an NRPS may have either a naturally-occurring sequence or a modified sequence. In another embodiment, the daptomycin NRPS or portion thereof is endogenous to the host cell, e.g., the host cell is S. roseosporus. A naturally-occurring or modified NRPS, or a domain, module or subunit thereof may be introduced into the host cell comprising the daptomycin NRPS or portion thereof. The heterologous domains, modules, subunits or NRPS may comprise a constitutive or regulatable promoter, which are known to those having ordinary skill in the art. The promoter can be either homologous or heterologous to the nucleic acid molecule being introduced into the cell. In one embodiment, the promoter may be from the daptomycin biosynthetic gene cluster, as described above.

The nucleic acid molecule comprising the NRPS or portion thereof (e.g., a domain, module or subunit) may be maintained episomally or integrated into the genome. The nucleic acid molecule may be introduced into the genome at, e.g., phage integration sites. Further, the nucleic acid molecule may be introduced into the genome at the site of an endogenous or heterologous NRPS or portion thereof or elsewhere in the genome. The nucleic acid molecule may be introduced in such a way to disrupt all or part of the function of a domain, module or subunit of an NRPS already present in the genome, or may be introduced in a manner that does not disturb the function of the NRPS or portion thereof.

The peptides produced by these NRPS may be useful as new compounds or may be useful in producing new compounds. In a preferred embodiment, the new compounds are useful as or may be used to produce antibiotic compounds. In another preferred embodiment, the new compounds are useful as or may be used to produce other peptides having useful activities, including but not limited to antibiotic, antifungal, antiviral, antiparasitic, antimitotic, cytostatic, antitumor, immuno-modulatory, anti-cholesterolemic, siderophore, agrochemical (e.g., insecticidal) or physicochemical (e.g., surfactant) properties. In a more preferred embodiment, the compounds produced using an altered NRPS polypeptide may be used in the synthesis of daptomycin-related compounds, including those described in U.S. application Ser. Nos. 09/738,742, 09/737,908 and 09/739,535, filed Dec. 15, 2000.

In addition, diverse variants of non-ribosomally synthesized peptides and polyketides may be achieved by altering the pools of available substrates during host cell cultivation. Commercial production of daptomycin, for example, is the result of cultivating the daptomycin producer Streptomyces roseosporus in the presence of decanoic acid, which alters the lipopeptide profile of the final products. See, e.g., U.S. Pat. No. 4,885,243. The feeding of N-acetyl cysteamine (SNAC) analogs of polyketide intermediates resulted in substantial increases in incorporation of the intermediates into the polyketide, when compared to the free carboxylic acid or ester analogs. See, e.g., Yue et al., J. Am. Chem. Soc., 109, pp. 1253-1255 (1987); Cane and Yang, J. Am. Chem. Soc., 109, 1255-1257 (1987); Cane et al., J. Am. Chem. Soc., 115, pp. 522-526 and 527-535 (1993); Cane et al., J. Am. Chem. Soc., 117, pp. 633-634 (1995); Pieder et al., J. Am. Chem. Soc., 117, pp. 11373-11374 (1995); each of which is incorporated herein by reference in its entirety. SNAC analogs of amino acids have been incorporated into a NRPS in vitro. Ehmann et al., Chem. Biol., 7, pp. 765-772 (2000). Thus it should be possible to feed SNAC or other pantetheine mimics to incorporate unnatural substrates into a NRPS-produced peptide.

Further diversity of non-ribosomally synthesized peptides and polyketides may also be achieved by expressing one or more NRPS and PKS genes (encoding natural, hybrid or otherwise altered modules or domains) in heterologous host cells, i.e., in host cells other than those from which the NRPS and PKS genes or modules originated.

In addition, one may express an ABC transporter or other polypeptide involved in antibiotic resistance in order to increase the resistance of a bacterial cell to daptomycin or a related compound. The ABC transporter may be overexpressed in an autologous cell (i.e., a cell that comprises the gene) or may be expressed in a heterologous cell (i.e., a cell that normally does not have the gene). Further, one may express an ABC transporter gene of the invention or another polypeptide involved in antibiotic resistance described herein in order to be able to select cells that are resistant to daptomycin. This selection may be useful for determining mechanisms of daptomycin resistance or may be used in standard molecular biological techniques in which antibody resistance is selected for.

Compounds of the Invention, Pharmaceutical Compositions Thereof and Methods of Treating Using Compounds and Compositions

Another object of the instant invention is to provide peptides or lipopeptides that may be produced by using the thioesterases, an NRPS or subunits thereof of the instant invention, as well as salts, esters, amides, ethers and protected forms thereof, and pharmaceutical formulations comprising these peptides, lipopeptides or their salts. In a preferred embodiment, the lipopeptide is daptomycin or a daptomycin-related lipopeptide, as described supra.

One may determine whether a peptide, lipopeptide or other compound of this invention has antibiotic activity using any of a variety of routine and well-known protocols in the art. One may use either an isolated or purified compound or may use an unpurified compound that is present in, e.g., fermentation culture broth or in a cell lysate. One may use either or both a gram-positive or a gram-negative bacterial test strain, and may use a variety of test strains to determine efficacy. In a preferred embodiment, the bacterial test strain will be a gram-positive test strain. In a more preferred embodiment, the bacterial test strain will be a Staphylococcus, more preferably S. aureus. An example of methods that can be used to determine antibiotic activity are provided in U.S. Pat. Nos. 4,208,408 and 4,537,717. One having ordinary skill in the art will recognize that other potential antibiotics and other test strains may be used.

Peptides, lipopeptides or pharmaceutically acceptable salts thereof can be formulated for oral, intravenous, intramuscular, subcutaneous, aerosol, topical or parenteral administration for the therapeutic or prophylactic treatment of diseases, particularly bacterial infections. In a preferred embodiment, the lipopeptide is daptomycin or a daptomycin-related lipopeptide. Reference herein to “daptomycin,” “daptomycin-related lipopeptide” or “lipopeptide” includes pharmaceutically acceptable salts thereof. Peptides, including daptomycin or daptomycin-related lipopeptides, can be formulated using any pharmaceutically acceptable carrier or excipient that is compatible with the peptide or with the lipopeptide of interest. See, e.g., Handbook of Pharmaceutical Additives: An International Guide to More than 6000 Products by Trade Name, Chemical, Function, and Manufacturer, Ashgate Publishing Co., eds., M. Ash and I. Ash, 1996; The Merck Index: An Encyclopedia of Chemicals, Drugs and Biologicals, ed. S. Budavari, annual; Remington's Pharmaceutical Sciences, Mack Publishing Company, Easton, Pa.; Martindale: The Complete Drug Reference, ed. K. Parfitt, 1999; and Goodman & Gilman's The Pharmaceutical Basis of Therapeutics, Pergamon Press, New York, N.Y., ed. L. S. Goodman et al.; the contents of which are incorporated herein by reference, for a general description of the methods for administering various antimicrobial agents for human therapy. Peptides or lipopeptides of this invention can be mixed with conventional pharmaceutical carriers and excipients and used in the form of tablets, capsules, elixirs, suspensions, syrups, wafers, creams and the like. Peptides or lipopeptides may be mixed with other therapeutic agents and antibiotics, such as discussed herein. The compositions comprising a compound of this invention will contain from about 0.1 to about 90% by weight of the active compound, and more generally from about 10 to about 30%.

The compositions of the invention can be delivered using controlled (e.g., capsules) or sustained release delivery systems (e.g., bioerodable matrices). Exemplary delayed release delivery systems for drug delivery that are suitable for administration of the compositions of the invention are described in U.S. Pat. Nos. 4,452,775 (issued to Kent), 5,239,660 (issued to Leonard), 3,854,480 (issued to Zaffaroni).

The compositions may contain common carriers and excipients, such as corn starch or gelatin, lactose, sucrose, microcrystalline cellulose, kaolin, mannitol, dicalcium phosphate, sodium chloride and alginic acid. The compositions may contain croscarmellose sodium, microcrystalline cellulose, corn starch, sodium starch glycolate and alginic acid.

Tablet binders that can be included are acacia, methylcellulose, sodium carboxymethylcellulose, polyvinylpyrrolidone (Povidone), hydroxypropyl methylcellulose, sucrose, starch and ethylcellulose.

Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. It may also be desirable to add a coloring agent to make the dosage form more aesthetic in appearance or to help identify the product.

For oral use, solid formulations such as tablets and capsules are particularly useful. Sustained release or enterically coated preparations may also be devised. For pediatric and geriatric applications, suspensions, syrups and chewable tablets are especially suitable. For oral administration, the pharmaceutical compositions are in the form of, for example, a tablet, capsule, suspension or liquid. The pharmaceutical composition is preferably made in the form of a dosage unit containing a therapeutically-effective amount of the active ingredient. Examples of such dosage units are tablets and capsules. For therapeutic purposes, the tablets and capsules which can contain, in addition to the active ingredient, conventional carriers such as binding agents, for example, acacia gum, gelatin, polyvinylpyrrolidone, sorbitol, or tragacanth; fillers, for example, calcium phosphate, glycine, lactose, maize-starch, sorbitol, or sucrose; lubricants, for example, magnesium stearate, polyethylene glycol, silica, or talc; disintegrants, for example, potato starch, flavoring or coloring agents, or acceptable wetting agents. Oral liquid preparations generally are in the form of aqueous or oily solutions, suspensions, emulsions, syrups or elixirs may contain conventional additives such as suspending agents, emulsifying agents, non-aqueous agents, preservatives, coloring agents and flavoring agents. Oral liquid preparations may comprise lipopeptide micelles or monomeric forms of the lipopeptide. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For intravenous (IV) use, a water soluble form of the peptide or lipopeptide can be dissolved in any of the commonly used intravenous fluids and administered by infusion. Intravenous formulations may include carriers, excipients or stabilizers including, without limitation, calcium, human serum albumin, citrate, acetate, calcium chloride, carbonate, and other salts. Intravenous fluids include, without limitation, physiological saline or Ringer's solution. Peptides or lipopeptides also may be placed in injectors, cannulae, catheters and lines.

Formulations for parenteral administration can be in the form of aqueous or non-aqueous isotonic sterile injection solutions or suspensions. These solutions or suspensions can be prepared from sterile powders or granules having one or more of the carriers mentioned for use in the formulations for oral administration. Lipopeptide micelles may be particularly desirable for parenteral administration. The compounds can be dissolved in polyethylene glycol, propylene glycol, ethanol, corn oil, benzyl alcohol, sodium chloride, and/or various buffers. For intramuscular preparations, a sterile formulation of a lipopeptide compound or a suitable soluble salt form of the compound, for example the hydrochloride salt, can be dissolved and administered in a pharmaceutical diluent such as Water-for-Injection (WFI), physiological saline or 5% glucose.

Injectable depot forms may be made by forming microencapsulated matrices of the compound in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in microemulsions that are compatible with body tissues.

For topical use the compounds of the present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient. For topical preparations, a sterile formulation of daptomycin, daptomycin-related lipopeptide or suitable salt forms thereof, may be administered in a cream, ointment, spray or other topical dressing. Topical preparations may also be in the form of bandages that have been impregnated with daptomycin or a daptomycin-related lipopeptide composition.

For application to the eyes or ears, the compounds of the present invention can be presented in liquid or semi-liquid form formulated in hydrophobic or hydrophilic bases as ointments, creams, lotions, paints or powders.

For rectal administration the compounds of the present invention can be administered in the form of suppositories admixed with conventional carriers such as cocoa butter, wax or other glycerides.

For aerosol preparations, a sterile formulation of the peptide or lipopeptide or salt form of the compound may be used in inhalers, such as metered dose inhalers, and nebulizers. A sterile formulation of a lipopeptide micelle may also be used for aerosol preparation. Aerosolized forms may be especially useful for treating respiratory infections, such as pneumonia and sinus-based infections.

Alternatively, the compounds of the present invention can be in powder form for reconstitution in the appropriate pharmaceutically acceptable carrier at the time of delivery. In one embodiment, the unit dosage form of the compound can be a solution of the compound or a salt thereof, in a suitable diluent in sterile, hermetically sealed ampules. The concentration of the compound in the unit dosage may vary, e.g. from about 1 percent to about 50 percent, depending on the compound used and its solubility and the dose desired by the physician. If the compositions contain dosage units, each dosage unit preferably contains from 50-500 mg of the active material. For adult human treatment, the dosage employed preferably ranges from 100 mg to 3 g, per day, depending on the route and frequency of administration.

In a further aspect, this invention provides a method for treating an infection, especially those caused by gram-positive bacteria, in humans and other animals. The term “treating” is used to denote both the prevention of an infection and the control of an established infection after the host animal has become infected. An established infection may be one that is acute or chronic. The method comprises administering to the human or other animal an effective dose of a compound of this invention. An effective dose of daptomycin, for example, is generally between about 0.1 and about 25 mg/kg daptomycin, daptomycin-related lipopeptide or pharmaceutically acceptable salts thereof. The daptomycin or daptomycin-related lipopeptide may be monomeric or may be part of a lipopeptide micelle. A preferred dose is from about 1 to about 25 mg/kg of daptomycin or daptomycin-related lipopeptide or pharmaceutically acceptable salts thereof. A more preferred dose is from about 1 to 12 mg/kg daptomycin or a pharmaceutically acceptable salt thereof. These dosages for daptomycin may be used as a starting point by one of skill in the art to determine and optimize effective dosages of other linear and cyclic peptides produced by the modified NRPS complexes of the invention.

In one embodiment, the invention provides a method for treating an infection, especially those caused by gram-positive bacteria, in a subject with a therapeutically-effective amount of modified daptomycin or other antibacterial peptide or lipopeptide produced by a modified NRPS of the invention. The daptomycin or antibacterial peptide or lipopeptide may be monomeric or in a lipopeptide micelle. Exemplary procedures for delivering an antibacterial agent are described in U.S. Pat. No. 5,041,567, issued to Rogers and in PCT patent application number EP94/02552 (publication no. WO 95/05384), the entire contents of which documents are incorporated in their entirety herein by reference. As used herein the phrase “therapeutically-effective amount” means an amount of modified daptomycin or other antibacterial peptide or lipopeptide produced by a modified NRPS according to the present invention, that prevents the onset, alleviates the symptoms, or stops the progression of a bacterial infection. The term “treating” is defined as administering, to a subject, a therapeutically-effective amount of a compound of the invention, both to prevent the occurrence of an infection and to control or eliminate an infection. The term “subject”, as described herein, is defined as a mammal, a plant or a cell culture. In a preferred embodiment, a subject is a human or other animal patient in need of peptide or lipopeptide compound treatment.

The peptide or lipopeptide antibiotic compound can be administered as a single daily dose or in multiple doses per day. The treatment regime may require administration over extended periods of time, e.g., for several days or for from two to four weeks. The amount per administered dose or the total amount administered will depend on such factors as the nature and severity of the infection, the age and general health of the patient, the tolerance of the patient to the antibiotic and the microorganism or microorganisms involved in the infection. A method of administration is disclosed in U.S. Ser. No. 09/406,568, filed Sep. 24, 1999, herein incorporated by reference, which claims the benefit of U.S. Provisional Application Nos. 60/101,828, filed Sep. 25, 1998, and 60/125,750, filed Mar. 24, 1999.

The methods of the present invention comprise administering modified daptomycin or other peptide or lipopeptide antibiotics, or pharmaceutical compositions thereof to a patient in need thereof in an amount that is efficacious in reducing or eliminating the gram-positive bacterial infection. The antibiotic may be administered orally, parenterally, by inhalation, topically, rectally, nasally, buccally, vaginally, or by an implanted reservoir, external pump or catheter. The antibiotic may be prepared for opthalmic or aerosolized uses. Modified daptomycin, a peptide or lipopeptide antibiotic produced by a modified NRPS of the invention, or a pharmaceutical compositions thereof, also may be directly injected or administered into an abscess, ventricle or joint. Parenteral administration includes subcutaneous, intravenous, intramuscular, intra-articular, intra-synovial, cisternal, intrathecal, intrahepatic, intralesional and intracranial injection or infusion. In a preferred embodiment, daptomycin or another peptide or lipopeptide is administered intravenously, subcutaneously or orally.

The method of the instant invention may be used to treat a patient having a bacterial infection in which the infection is caused or exacerbated by any type of gram-positive bacteria. In a preferred embodiment, modified daptomycin, daptomycin-related lipopeptide, or another peptide or lipopeptide antibiotic produced by a modified NRPS of the invention, or pharmaceutical compositions thereof, are administered to a patient according to the methods of this invention. In another preferred embodiment, the bacterial infection may be caused or exacerbated by bacteria including, but not limited to, methicillin-susceptible and methicillin-resistant staphylococci (including Staphylococcus aureus, Staphylococcus epidermidis, Staphylococcus haemolyticus, Staphylococcus hominis, Staphylococcus saprophyticus, and coagulase-negative staphylococci), glycopeptide intermediary-susceptible Staphylococcus aureus (GISA), penicillin-susceptible and penicillin-resistant streptococci (including Streptococcus pneumoniae, Streptococcus pyogenes, Streptococcus agalactiae, Streptococcus avium, Streptococcus bovis, Streptococcus lactis, Streptococcus sangius and Streptococci Group C, Streptococci Group G and viridans streptococci), enterococci (including vancomycin-susceptible and vancomycin-resistant strains such as Enterococcus faecalis and Enterococcus faecium), Clostridium difficile, Clostridium clostridiiforme, Clostridium innocuum, Clostridium perfringens, Clostridium ramosum, Haemophilus influenzae, Listeria monocytogenes, Corynebacterium jeikeium, Bifidobacterium spp., Eubacterium aerofaciens, Eubacterium lentum, Lactobacillus acidophilus, Lactobacillus casei, Lactobacilllus plantarum, Lactococcus spp., Leuconostoc spp., Pediococcus, Peptostreptococcus anaerobius, Peptostreptococcus asaccarolyticus, Peptostreptococcus magnus, Peptostreptococcus micros, Peptostreptococcus prevotii, Peptostreptococcus productus, Propionibacterium acnes, and Actinomyces spp.

The antibacterial activity of daptomycin against classically “resistant” strains is comparable to that against classically “susceptible” strains in in vitro experiments. In addition, the minimum inhibitory concentration (MIC) value for daptomycin against susceptible strains is typically 4-fold lower than that of vancomycin. Thus, in a preferred embodiment, modified daptomycin, daptomycin-related lipopeptide antibiotic, a peptide or lipopeptide antibiotic produced by the modified NRPS of the invention, or pharmaceutical compositions thereof, are administered according to the methods of this invention to a patient who exhibits a bacterial infection that is resistant to other antibiotics, including vancomycin. In addition, unlike glycopeptide antibiotics, daptomycin exhibits rapid, concentration-dependent bactericidal activity against gram-positive organisms. Thus, in a preferred embodiment, daptomycin, a lipopeptide antibiotic, or pharmaceutical compositions thereof are administered according to the methods of this invention to a patient in need of rapidly acting antibiotic therapy.

The method of the instant invention may be used for a gram-positive bacterial infection of any organ or tissue in the body. These organs or tissue include, without limitation, skeletal muscle, skin, bloodstream, kidneys, heart, lung and bone. The method of the invention may be used to treat, without limitation, skin and soft tissue infections, bacteremia and urinary tract infections. The method of the invention may be used to treat community acquired respiratory infections, including, without limitation, otitis media, sinusitis, chronic bronchitis and pneumonia, including pneumonia caused by drug-resistant Streptococcus pneumoniae or Haemophilus influenzae. The method of the invention also may be used to treat mixed infections that comprise different types of gram-positive bacteria, or which comprise both gram-positive and gram-negative bacteria, including aerobic, caprophilic or anaerobic bacteria. These types of infections include intra-abdominal infections and obstetrical/gynecological infections. The methods of the invention may be used in step-down therapy for hospital infections, including, without limitation, pneumonia, intra-abdominal sepsis, skin and soft tissue infections and bone and joint infections. The method of the invention also may be used to treat an infection including, without limitation, endocarditis, nephritis, septic arthritis and osteomyelitis. In a preferred embodiment, any of the above-described diseases may be treated using daptomycin, lipopeptide antibiotic, or pharmaceutical compositions thereof. Further, the diseases may be treated using daptomycin or lipopeptide antibiotic in either a monomeric or micellar form.

Modified daptomycin, daptomycin-related lipopeptide, or another peptide or lipopeptide produced by a modified NRPS according to the invention, may also be administered in the diet or feed of a patient or animal. If administered as part of a total dietary intake, the amount of modified daptomycin or other peptide or lipopeptide can be less than 1% by weight of the diet and preferably no more than 0.5% by weight. The diet for animals can be normal foodstuffs to which modified daptomycin or the other peptide or lipopeptide can be added or it can be added to a premix.

The method of the instant invention may also be practiced while concurrently administering one or more antifungal agents and/or one or more antibiotics other than modified daptomycin or other peptide or lipopeptide antibiotic. Co-administration of an antifungal agent and an antibiotic other than modified daptomycin or another peptide or lipopeptide antibiotic may be useful for mixed infections such as those caused by different types of gram-positive bacteria, those caused by both gram-positive and gram-negative bacteria, or those that caused by both bacteria and fungus. Furthermore, modified daptomycin or other peptide or lipopeptide antibiotic may improve the toxicity profile of one or more co-administered antibiotics. It has been shown that administration of daptomycin and an aminoglycoside may ameliorate renal toxicity caused by the aminoglycoside. In a preferred embodiment, an antibiotic and/or antifungal agent may be administered concurrently with modified daptomycin, other peptide or lipopeptide antibiotic, or in pharmaceutical compositions comprising modified daptomycin or another peptide or lipopeptide antibiotic.

Antibacterial agents and classes thereof that may be co-administered with modified daptomycin or other peptide or lipopeptide antibiotics include, without limitation, penicillins and related drugs, carbapenems, cephalosporins and related drugs, aminoglycosides, bacitracin, gramicidin, mupirocin, chloramphenicol, thiamphenicol, fusidate sodium, lincomycin, clindamycin, macrolides, novobiocin, polymyxins, rifamycins, spectinomycin, tetracyclines, vancomycin, teicoplanin, streptogramins, anti-folate agents including sulfonamides, trimethoprim and its combinations and pyrimethamine, synthetic antibacterials including nitrofurans, methenamine mandelate and methenamine hippurate, nitroimidazoles, quinolones, fluoroquinolones, isoniazid, ethambutol, pyrazinamide, para-aminosalicylic acid (PAS), cycloserine, capreomycin, ethionamide, prothionamide, thiacetazone, viomycin, eveminomycin, glycopeptide, glycylcylcline, ketolides, oxazolidinone; imipenen, amikacin, netilmicin, fosfomycin, gentamicin, ceftriaxone, Ziracin, LY 333328, CL 331002, HMR 3647, Linezolid, Synercid, Aztreonam, and Metronidazole, Epiroprim, OCA-983, GV-143253, Sanfetrinem sodium, CS-834, Biapenem, A-99058.1, A-165600, A-179796, KA 159, Dynemicin A, DX8739, DU 6681; Cefluprenam, ER 35786, Cefoselis, Sanfetrinem celexetil, HGP-31, Cefpirome, HMR-3647, RU-59863, Mersacidin, KP 736, Rifalazil; Kosan, AM 1732, MEN 10700, Lenapenem, BO 2502A, NE-1530, PR 39, K130, OPC 20000, OPC 2045, Veneprim, PD 138312, PD 140248, CP 111905, Sulopenem, ritipenam acoxyl, RO-65-5788, Cyclothialidine, Sch-40832, SEP-132613, micacocidin A, SB-275833, SR-15402, SUN A0026, TOC 39, carumonam, Cefozopran, Cefetamet pivoxil, and T 3811.

In a preferred embodiment, antibacterial agents that may be co-administered with modified daptomycin or peptide or lipopeptide antibiotic produced by a modified NRPS according to this invention include, without limitation, imipenen, amikacin, netilmicin, fosfomycin, gentamicin, ceftriaxone, teicoplanin, Ziracin, LY 333328, CL 331002, HMR 3647, Linezolid, Synercid, Aztreonam, and Metronidazole.

Antifungal agents that may be co-administered with modified daptomycin or other peptide or lipopeptide antibiotic include, without limitation, Caspofungen, Voriconazole, Sertaconazole, IB-367, FK-463, LY-303366, Sch-56592, Sitafloxacin, DB-289 polyenes, such as Amphotericin, Nystatin, Primaricin; azoles, such as Fluconazole, Itraconazole, and Ketoconazole; allylamines, such as Naftifine and Terbinafine; and anti-metabolites such as Flucytosine. Other antifungal agents include without limitation, those disclosed in Fostel et al., Drug Discovery Today 5:25-32 (2000), herein incorporated by reference. Fostel et al. disclose antifungal compounds including Corynecandin, Mer-WF3010, Fusacandins, Artrichitin/LL 15G256γ, Sordarins, Cispentacin, Azoxybacillin, Aureobasidin and Khafrefungin.

Modified daptomycin or other peptide or lipopeptide antibiotics, including daptomycin-related lipopeptides, may be administered according to this method until the bacterial infection is eradicated or reduced. In one embodiment, modified daptomycin, or other peptide or lipopeptide produced by a modified NRPS according to the invention, is administered for a period of time from 3 days to 6 months. In a preferred embodiment, modified daptomycin, or other peptide or lipopeptide, is administered for 7 to 56 days. In a more preferred embodiment, modified daptomycin, or other peptide or lipopeptide is administered for 7 to 28 days. In an even more preferred embodiment, modified daptomycin or other peptide or lipopeptide antibiotic is administered for 7 to 14 days. In another embodiment, the antibiotic is administered for 3 to 7 days. Modified daptomycin, or other peptide or lipopeptide produced by a modified NRPS according to the invention, according to the invention may be administered for a longer or shorter time period if it is so desired.

In order that this invention may be more fully understood, the following examples are set forth. These examples are for the purpose of illustration only and are not to be construed as limiting the scope of the invention in any way.

### EXAMPLE 1

### Initial Sequencing of the Streptomyces roseosporus Daptomycin Biosynthetic Gene Cluster

Streptomyces roseosporus strain A21978.6 (American Type Culture Collection Accession No. 31568) was used for the construction of a cosmid library. Genomic DNA was digested partially with Sau3A1 and alkaline phosphatase (Boehringer Mannheim Biochemicals). DNA of approximately 40 kb in length was isolated and ligated to BamHI-digested cosmid pKC1471 and packaged with a Gigapack packaging extract (Stratagene, Inc.) as described in Hosted and Baltz, J. Bacteriol., 179, pp. 180-186 (1997). Packaged DNA was introduced into E. coli XL1-Blue-MFR (Stratagene, Inc.) and individual clones containing cosmid DNA were stored as an ordered array in a 96-well dot blot apparatus. Twelve cultures from a row of microtiter wells were pooled, and screened by hybridization to a 2.1-kB SphI fragment of DNA from plasmid pRHB153 and to a 5.2-kB DraI-KpnI fragment from pRHB157, both containing NRPS sequences cloned from S. roseosporus (see McHenney et al., supra). Individual cosmids from the hybridizing pools were identified by hybridization to the same probes.

Cosmid and plasmid DNA was hydrodynamically sheared and then separated by electrophoresis on a standard 1% agarose gel. The separated DNA fragments 2500-3000 bp in length were excised from the gel and purified by the GeneClean™ procedure (BIO 101, Inc.). The ends of the gel-purified DNA fragments were then filled in or made blunt using T4 DNA polymerase. The DNA fragments were ligated to unique BstXI-linker adapters (5′-GTCTTCACCACGGGG-3′-SEQ ID NO: 13, and 5′GTGGTGAAGAC-3′-SEQ ID NO: 14, in 100-1000 fold molar excess). These linkers are complementary to the BstXI-cut pGTC vector (Genome Therapeutics Corp., Waltham, Mass.), while the overhang is not self-complementary. Therefore, the linkers will not concatemerize, nor will the open vector self-ligate easily. The linker-adapted inserts were separated from the unincorporated linkers by electrophoresis on a 1% agarose gel and purified using GeneClean™. The purified linker-adapted inserts were ligated to BstXI-cut pGTC vector to construct “shotgun” subclone libraries.

The pGTC library was then transformed into DH5α competent cells (Gibco/BRL, DH5α transformation protocol). Transformation was assessed by plating onto antibiotic plates containing ampicillin and IPTG/Xgal (IPTG=isopropyl-b-D-thiogalactopyranoside; Xgal=5-bromo-4-chloro-3-indoyl-b-D-thiogalactopyranoside.) The plates were incubated overnight at 37° C. Transformants were plate purified and the purified clones containing the following plasmids were picked for further analysis.

Plasmids pRHB160, containing an insert of approximately 50 kb of S. roseosporus DNA, pRHB613, containing an insert of approximately 15 kb, pRHB614, containing an insert of approximately 13 kb, and pRHB159, containing an insert of approximately 51 kb, were chosen for DNA sequencing. (See McHenney et al., supra).

Individual cultures of strains transformed with the above plasmids were grown overnight at 37° C. DNA was purified using a silica bead DNA preparation method (Engelstein et al., Microb. Comp. Genomics 3(4):237-241, 1998). In this manner, 25 mg of DNA were obtained per clone. These purified DNA samples were then sequenced using primarily ABI dye-terminator chemistry. All subsequent steps were based on sequencing by ABI377 or Amersham automated DNA sequencing methods according to the manufacturer's instructions. The ABI dye terminator sequence reads were run on either ABI377 or Amersham MegaBace™ capillary machines. The data were transferred to UNIX machines following lane tracking of the gels. Base calls and quality scores were determined using the program PHRED (Ewing et al., Genome Res. 8:175-185, 1998). Reads were assembled using PHRAP (P. Green, Abstracts of DOE Human Genome Program Contractor-Grantee Workshop V, January 1996, p. 157) with default program parameters and quality scores. The initial assembly was done at 6× coverage.

### EXAMPLE 2

### Isolation and Analysis of Additional DNA Molecules of the Streptomyces roseosporus Biosynthetic Gene Cluster

Mycelium for preparation of megabase DNA was obtained from overnight cultures of Streptomyces roseosporus (NRRL11379) (ATCC No. 31568) shaken in F10A broth (2% agar, 25% soluble starch, 0.2% dextrose, 0.5% yeast extract, 0.5% peptone and 0.3% calcium carbonate) at 30° C. Washed cells were embedded in Seakem™ GTG agarose (FMC Bioproducts, 1% final concentration), incubated in lysozyme (2 mg/mL TE) at 37° C. for 3 h, then lysed in 0.1×NLS+0.2 mg/mL proteinase K at 50° C. overnight to release DNA into the gel matrix. Agarose containing DNA was washed with 1 mM EDTA (pH 8) before treatment with BamHI at 37° C. Partially digested DNA was then subjected to a two-step size selection process in 0.6% agarose gels (in 0.5×TBE) by pulsed-field electrophoresis using a CHEF Mapper DRIII (Biorad) set at 6V/cm, 120° angle, 12° C. The first selection consisted of a 14 h run with a 22-44 sec linearly ramped switch time. Gel containing DNA co-migrating with 100-200 kb lambda concatamer size markers was excised and cast in a second gel for an 18 h run with a 3-5 sec linear ramp. DNA estimated at 75-145 kb relative to size markers was electroeluted (MiniProtean II Cell model, Biorad) in TAE.

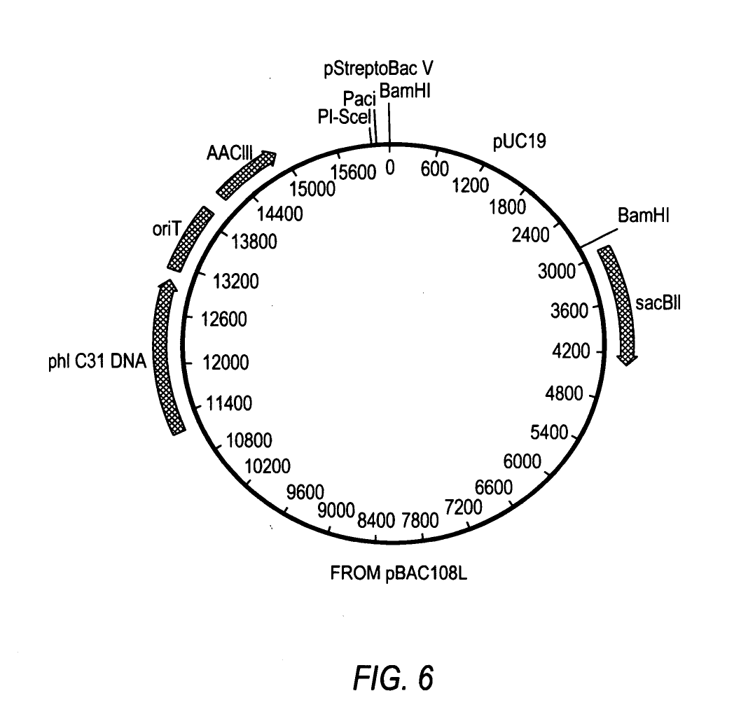

The single-copy BAC library cloning vector pStreptoBAC V is derived from pBACe3.6 (Frengen et al., A modular, positive selection bacterial artificial chromosome vector with multiple cloning sites, Genomics, 58: 250-253 (1999)). The pBACe3.6 was modified to contain two markers, Amp^{R} for selection in E. coli and Apra^{R} for selection in Streptomyces, as well as oriT and attP sequences from the phage jC31 for conjugation and site specific integration in Streptomyces. See FIG. 6. To prepare the pStreptoBAC V vector for ligation with the S. roseosporus DNA, the vector was first digested with BamHI and the reaction was inactivated by heat (65° C. for 1 h). DNA was then dephosporylated with Shrimp Alkaline Phosphatase for 30 min. The two bands (13 kb and 3 kb corresponding to the pUC fragment) were separated on 0.6% agarose gel and the 13 kb band was purified using Geneclean spin columns.

200 ng of the S. roseosporus DNA was ligated to 75 ng of BamHI cut and phosphatased pStreptoBAC V vector DNA using 9 U of T4 DNA ligase (Promega) in a 150 μl reaction. After 16 h at 16° C., the ligations were heated at 65° C. for 30 min, dialyzed against 10% polyethylene glycol 8000, and transformed into 10 μl of DH10B electrocompetent cells (Gibco/BRL) using a cell porator with voltage booster (Gibco/BRL) at 300 V and 4 kΩ. Cells were plated on media (LB agar) containing 100 mg/mL apramycin and 5% sucrose. Analysis of sample clones showed a range of inserts from 39 kb to 105 kb. The mean insert size was 71.4 kb, with a standard deviation of 14.7 kb. Approximately 2,000 clones were archived at −80° C. in 96-well microtiter plates.

This BAC library was screened using the polymerase chain reaction (PCR) using primer pairs P61/P62, P72/P73 and P74/P75, shown below. Nucleotide positions refer to the numbering of SEQ ID NO: 1, and “C” indicates that the primer sequence corresponds to the complementary strand of SEQ ID NO: 1:

| | | SEQ | |
|---|---|---|---|
| | | ID | Nucleotide |
| Primer | Sequence | NO: | Position |
| P61 | GCTCGTCCCCCTCCCCGCACT | 137 | 41305-41325 |
| P62 | CGAACAGGTGGGCTTTGAGTGG | 138 | 41993-42014 (C) |
| P72 | CTTCGTGAACACCCTCGTCC | 139 | 82103-82122 |
| P73 | GTTCGTCGAGGTCCAGTACG | 140 | 83009-83028 (C) |
| P74 | GCACCAGCGTGTGCGGATCG | 141 | 92-111 |
| P75 | CACGTACGTGACGATCCTCG | 142 | 800-819 (C) |

PCR was performed under the following conditions: 94° C., 45 sec., 54° C., 30 sec., 72° C., 1 min. for 32 cycles. Taq polymerase, as well as the accessory reagents, were supplied by Gibco BRL (Bethesda); all reactions included 5% DMSO.

Clone B12:03A05 was initially detected with primer pair P61/P62 (see above), and subsequently confirmed as a positive hit with the other two primer pairs. DNA of clone B12:03A05 was obtained by standard alkaline lysis procedures and used for DNA sequencing (see below).

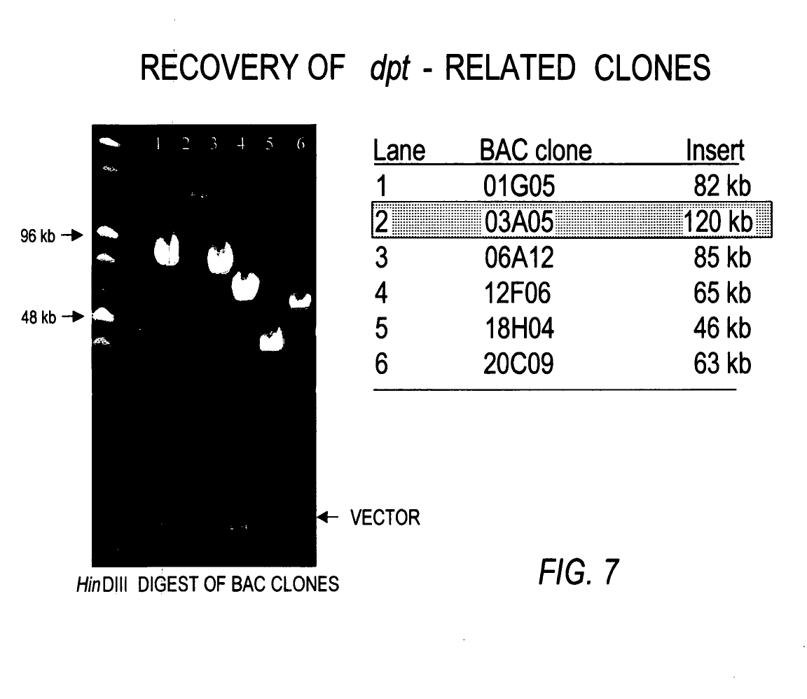

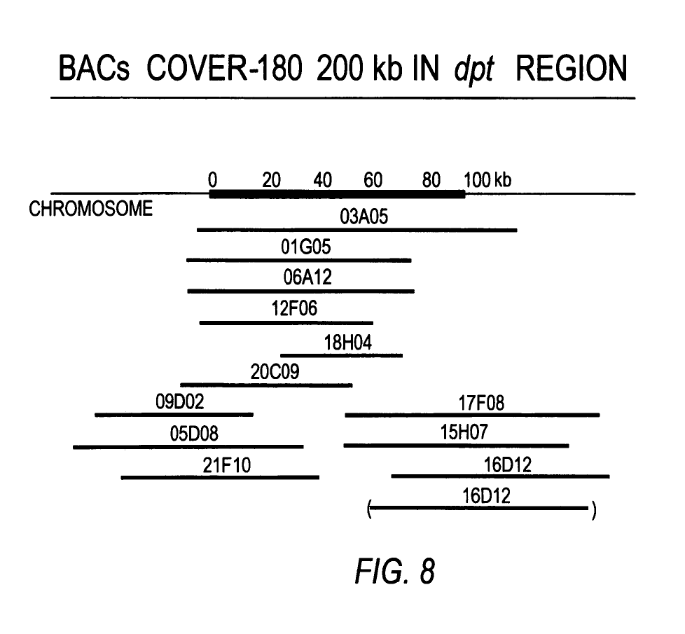

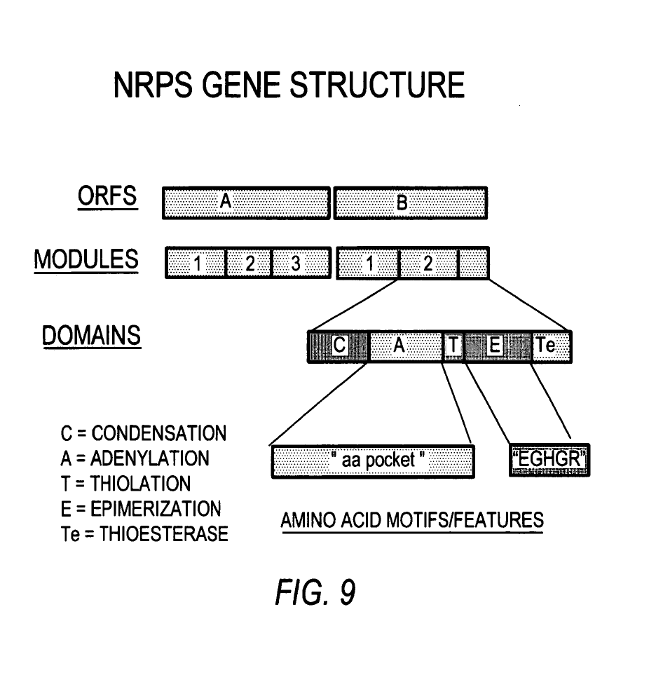

A number of other clones that encompass parts of the daptomycin gene cluster (dpt-related clones) were isolated from the BAC library. These clones include B12:01G05 (insert size 82 kb), B12:06A12 (insert size 85 kb), B12:12F06 (insert size 65 kb), B12:18H04 (insert size 46 kb) and B12:20C09 (insert size 65 kb). See FIG. 7, which shows a HinDIII digest of these BAC clones. Other BACs that were isolated in the daptomycin gene cluster region include B12:09D02, B12:17F08, B12:05D08, B12:15H07, B12:21F10 and B12:16D12. These BACS cover 180 to 200 kb. FIG. 8 shows the approximate location of the BAC clones relative to the daptomycin gene cluster.

Extension of the daptomycin biosynthetic gene cluster sequence determined in Example 2 was accomplished by sequencing 1 μg aliquots of BAC DNA from clone B12:03A05 using the ABI Prism Dye Terminator Cycle Sequencing Ready Reaction kit (Perkin Elmer), the manufacturer's recommended reaction mix and conditions, and the following primers (C indicates that the primer sequence corresponds to the complementary strand of SEQ ID NO: 1):

| | | SEQ | |
|---|---|---|---|
| | | ID | Nucleotide |
| Primer | Sequence | NO: | Position |
| P76 | CGTACTGGACCTCGACGACC | 143 | 83009-83028 |
| P78 | CGACCAGCGTGTGTACGTCC | 144 | 83609-83628 |
| P92 | AGTCCTCAGCCATCTCCTCG | 145 | 84584-84603 (C) |
| P84 | GAGACCGTCGGCGTGGACG | 146 | 84222-84240 |
| P95 | AGGGCCACACCGTCGAACTCC | 147 | 84709-84729 |
| P86 | ATCGTCGCCGACTACCTCGC | 148 | 84795-84814 |
| P96 | GGCAGCTACCTCGTACTGG | 149 | 85297-85315 |
| P97 | TGTACGACAGCGGCGTCGAAC | 150 | 85959-85979 |
| P101 | CGATTCTCGGCATGTTCGCC | 151 | 86636-86655 |
| P105 | TCGTCTCCTACATGACCTCG | 152 | 87194-87213 |
| P107 | TTCACGGAAACCGAACGTCG | 153 | 87864-87883 |
| P111 | GGTTCAGGCCGCAGCCAACG | 154 | 88468-88487 |
| P117 | CGCTGACCTTGGTCAGAAGCC | 155 | 89176-89196 |

Electrophanerograms were inspected and corrected as appropriate, and the sequences were aligned using the AssemblyLign Module of MacVector™. The aligned sequence (contig) was saved as a MacVector™ file for analysis and annotation. Identification of potential ORFs and potential stops/starts was performed using the open reading frames option in MacVector™.

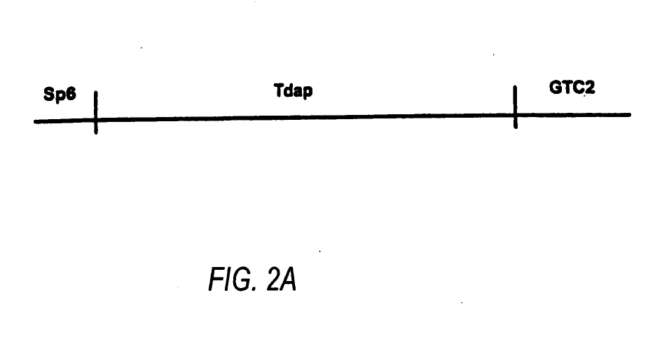

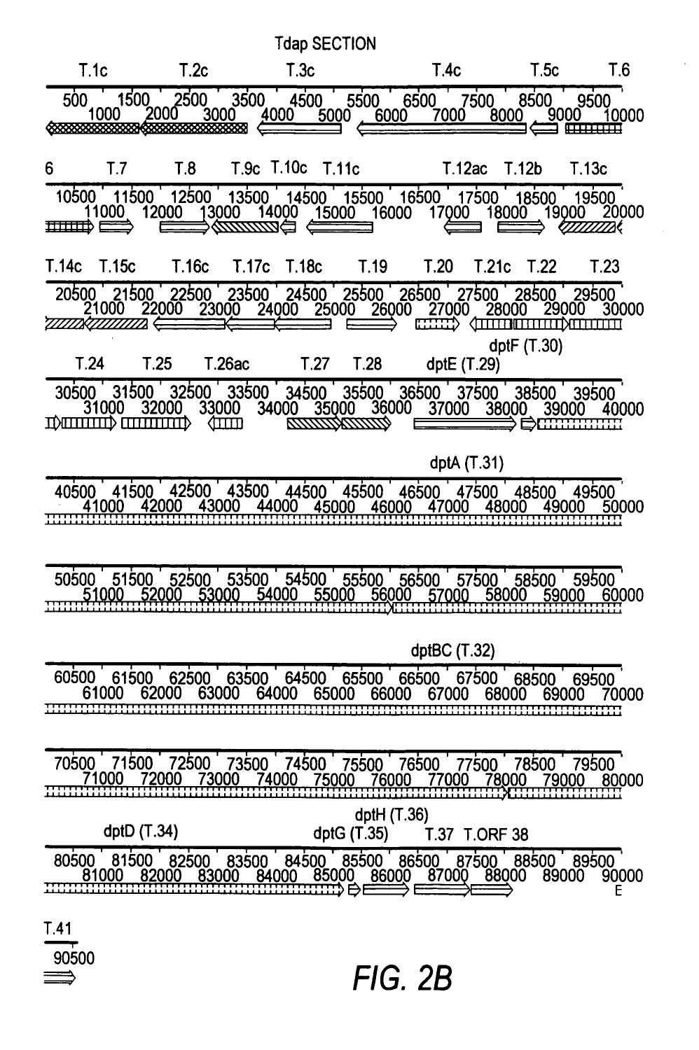

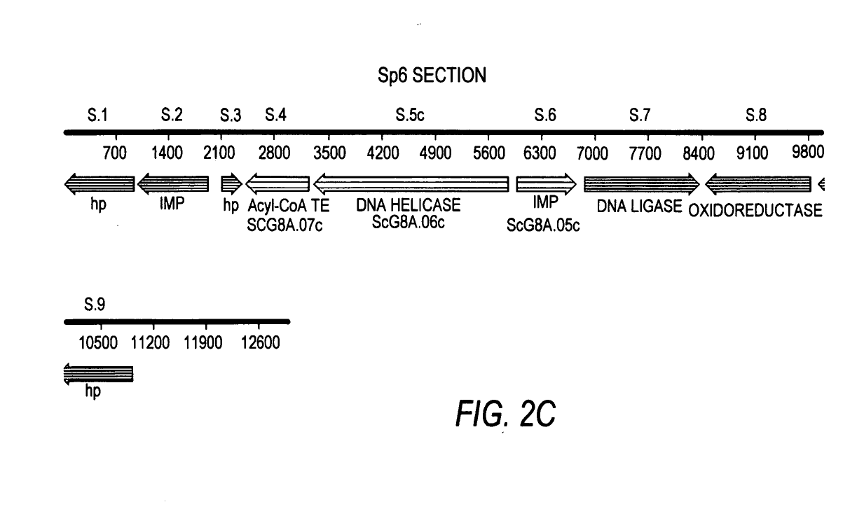

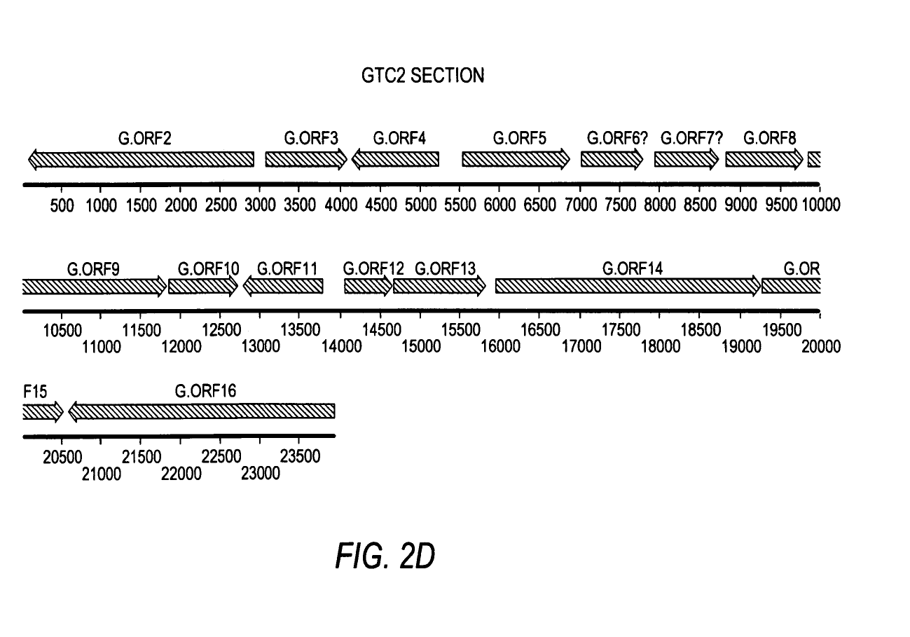

Analysis of the 90 kb sequence showed a total of 38 open reading frames in the daptomycin biosynthetic gene cluster region. See FIG. 2. The ORFs range in size from 228 basepairs (bp) to 22 kb. The three largest ORFs are NRPS genes, as discussed below. One of the NRPS genes were predicted to have thioesterase activity based on the presence of conserved motifs, GXSXG (see Example 3). Another predicted open reading frames also encodes a protein with thioesterase activity (see Example 3). A number of potential ABC transporters were also identified.

The sequence of the daptomycin biosynthetic gene cluster is shown in SEQ ID NO: 1. See also FIG. 2. The genes encoding the daptomycin non-ribosomal peptide synthetase (NRPS) are designated dptA, dptBC and dptD. We designate as a promoter region all sequences upstream from the start of an ORF of interest that are not part of an upstream ORF. Because dptA, dptBC and dptD have overlapping start and stop codons and apparently are translationally coupled (e.g., the TGA stop codon of dptBC overlaps with the ATG start codon of dptD, which is presumably associated with its own ribosome binding site), we thus indicate the promoter of the whole cluster (comprising dptE, dptF, dptA, dptBC and dptD) as the daptomycin NPRS promoter.

The DNA sequence of the ORF of the daptomycin NRPS dptA gene (nucleotides 38555-56047 of SEQ ID NO: 1) is shown in SEQ ID NO: 10. The ORF is 17493 nucleotides in length. The amino acid sequence of the encoded DptA protein is shown in SEQ ID NO: 9. The protein is 5830 amino acid residues in length.

The DNA sequence of the ORF of the daptomycin NRPS dptBC gene (nucleotides 56044-78060 of SEQ ID NO: 1) is shown in SEQ ID NO: 12. The ORF is 22017 nucleotides in length. The amino acid sequence of the encoded DptBC protein is shown in SEQ ID NO: 11. The protein is 7338 amino acid residues in length.

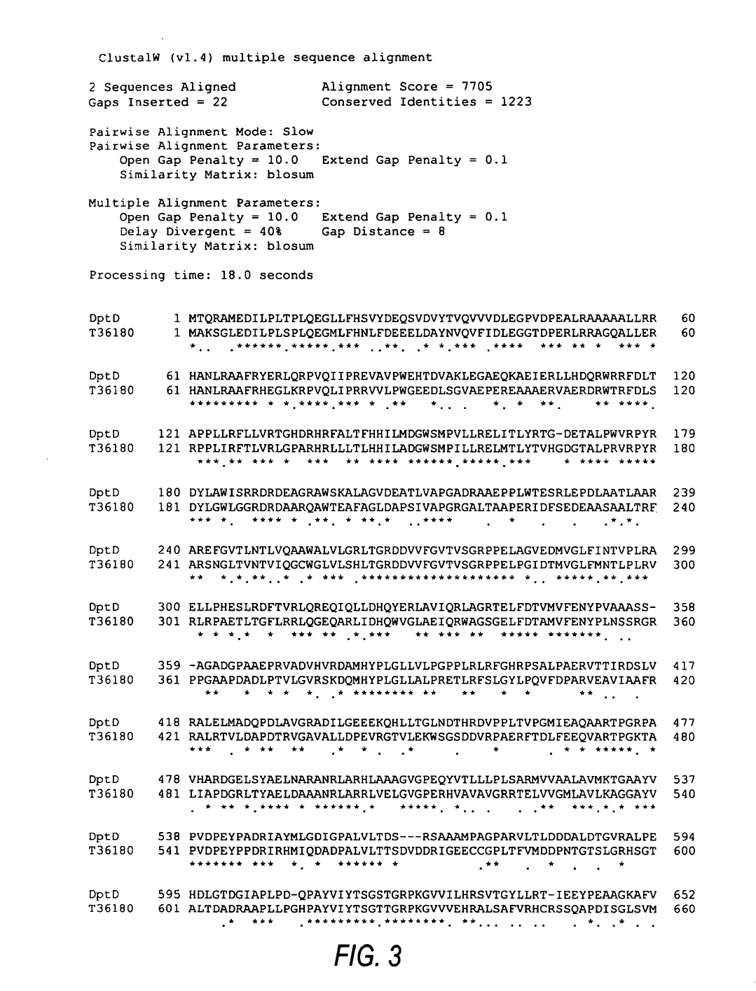

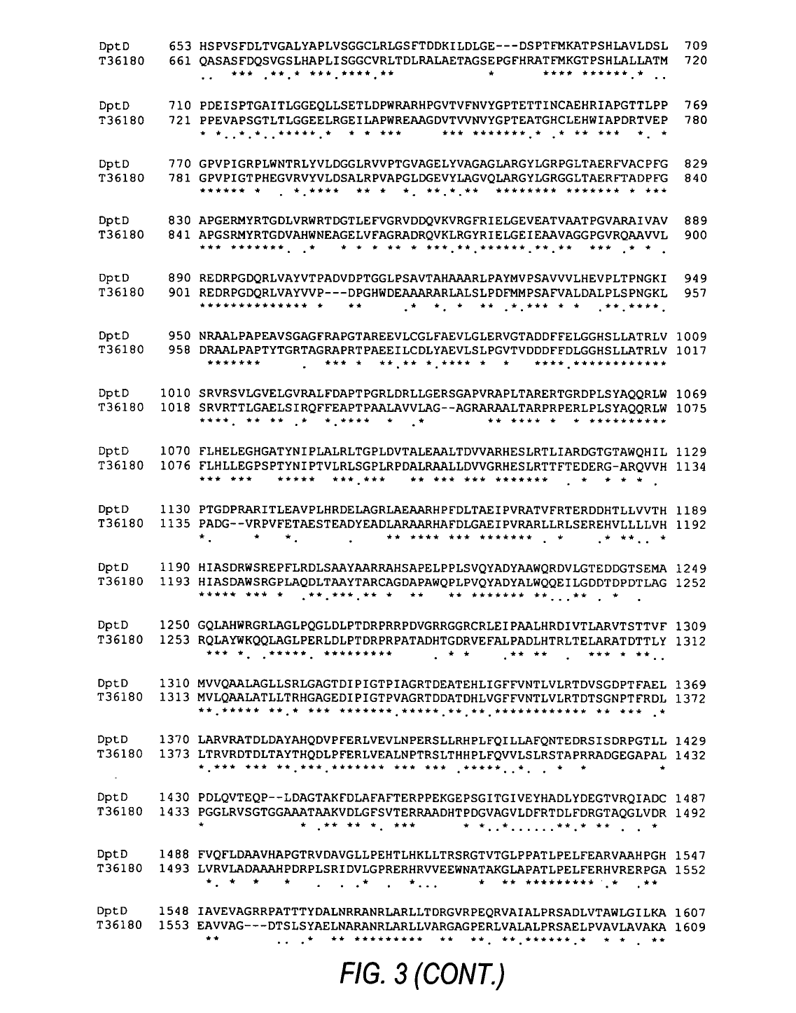

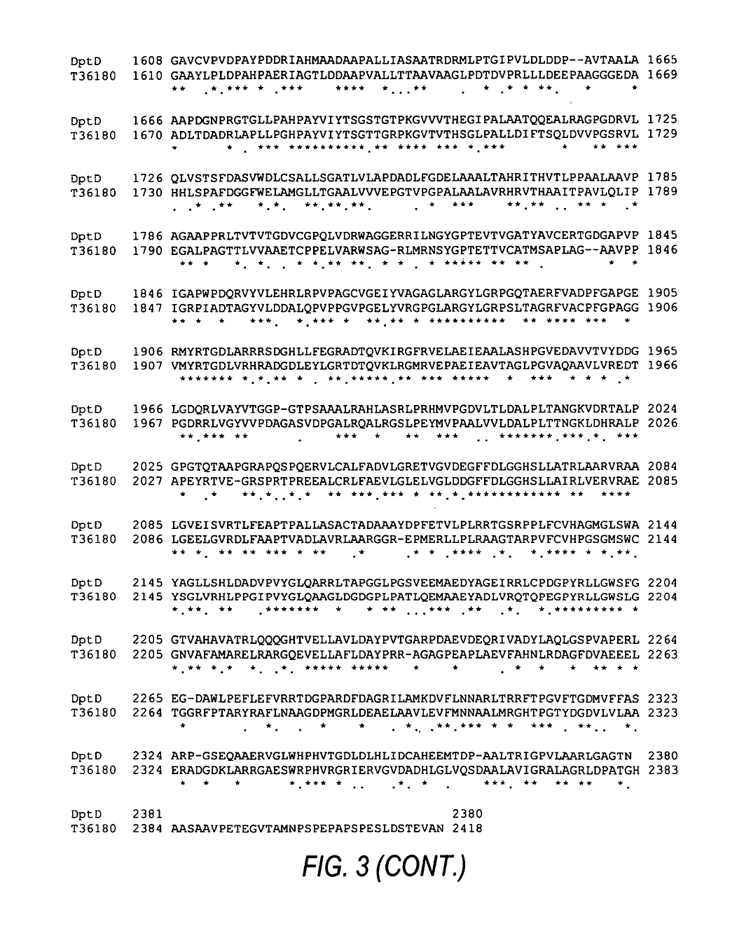

The DNA sequence of the ORF of the daptomycin NRPS dptD gene (nucleotides 78057-85196 of SEQ ID NO: 1) is shown in SEQ ID NO: 3. The ORF is 7140 nucleotides. The dptD gene ORF encodes a type I thioesterase (TEI) domain at the C-terminus. The amino acid sequence of the predicted DptD protein is shown in SEQ ID NO: 7 (see FIG. 3). The protein is 2379 amino acids in length

The dptE and dptF genes are located between dptA and the daptomycin NPRS promoter.

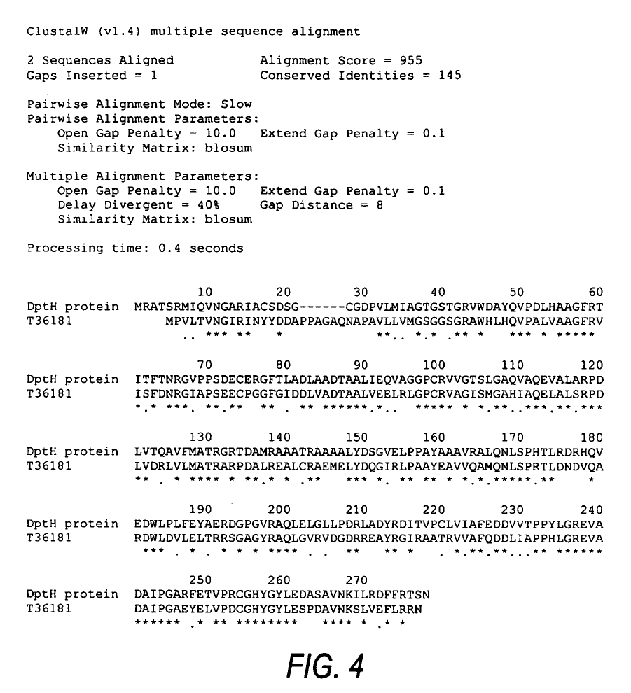

The DNA sequence of the dptH thioesterase-encoding gene is shown in SEQ ID NO: 4 (nucleotides 85498-86350 of SEQ ID NO: 1); the promoter region of dptH is shown in SEQ ID NO: 5 (nucleotides 85498-85534 of SEQ ID NO: 1); and the open reading frame of dptH is shown in SEQ ID NO: 6 (nucleotides 85535-86350 of SEQ ID NO: 1). The amino acid sequence of the predicted DptH protein is shown in SEQ ID NO: 8 (see FIG. 4).

The promoter region of the daptomycin NRPS (nucleotides 36018-36407 of SEQ ID NO: 1) is shown in SEQ ID NO: 2.

The sequence for the DNA downstream of the 90 kb contig was generated by Genome Therapeutics Corps. from plasmid pV107 by transposon primed sequencing using a system such as GPS-1 Genome priming system (New England Biolabs). Plasmid pV107 comprises an approximately 28 kb EcoRI fragment subcloned using standard techniques from B12:03A05 genome into the vector pNEB193, cut with EcoRI (New England Biolabs). The fragment is called the GTC2 fragment. An adequate number of transposon tagged library clones were sequenced to generate a 6-fold redundant contig, and additional local sequencing off PCR products was used to polish the sequence where needed. The overlap between the 5′ end of the contig and the existing 90 kb was removed from the contig, so that the beginning of the pV107 derived sequences (referred to as GTC2) starts with 1. The sequence of the GTC2 fragment is provided in SEQ ID NO: 106.

### EXAMPLE 3

### Identification of the dptD and dptH Genes as Thioesterases

Amino acid motifs typical of non-ribosomal peptide synthetases and thioesterases were identified by inspection of the dptD and dptH genes and predicted translation products thereof. The amino acid sequence motif GXSXG, wherein X is any one of the twenty L-amino acids that are inserted translationally into ribosomally produced proteins, is indicative of thioesterases (See Mootz et al., J. Bacteriol. 179:6843-6850, 1997, incorporated herein by reference in its entirety). SEQ ID NOs 7-8 were inspected for the GXSXG thioesterase motif. In SEQ ID NO:7, the amino acid sequence match to the thioesterase motif GWSFG (SEQ ID NO: 166) was found at coordinates 2200-2204, encoded by nucleotides 84654-84668 of SEQ ID NO:1. In SEQ ID NO:8, the amino acid sequence match to the thioesterase motif GTSLG (SEQ ID NO: 167) was found at coordinates 97-101, encoded by nucleotides 85823-85838 of SEQ ID NO:1.

The DptD protein of SEQ ID NO:7 was aligned to the CDA III protein of Streptomyces coelicolor. The alignment was performed using the Clustal W (v1.4) program in slow pairwise alignment mode. An open gap penalty of 10.0, an extend gap penalty of 0.1, and a blosum similarity matrix to the CDA III protein was used. The CDA III protein is a non-ribosomal peptide synthetase with a carboxy-terminal thioesterase domain (see GENBANK accession number AL035707, version AL035707.1 GI:4490978, hereby incorporated by reference in its entirety). The CDA III amino acid sequence used for the alignment was generated using the MacVector program by creating a contig from two GENBANK cosmid sequences, AL035707 and AL035640, and then translating the open reading frame in the contig annotated in GENBANK. The sequence comparison (FIG. 3) revealed an alignment score of 7705 and 1223 conserved identities, indicating significant similarity between the two compared sequences. The GXSXG thioesterase motifs of the DptD protein and the CDA III protein were aligned in this analysis.

The GXSXG thioesterase motif of the DptH protein of SEQ ID NO: 8 was aligned to the GXSXG thioesterase motif of the CDA III protein of Streptomyces coelicolor (CAA71338 protein, see above). The alignment was performed the Clustal W (v1.4) program in slow pairwise alignment mode. An open gap penalty of 10.0, an extend gap penalty of 0.1, and a blosum similarity matrix to the Streptomyces thioesterase protein of GENPEPT record CAA71338 (version CAA71338.1 GI:2647975, hereby incorporated by reference in its entirety) was used. The alignment (FIG. 4) revealed an alignment score of 955 and 145 conserved identities indicating significant similarity between the two compared sequences.

These analyses show that dptD and dptH encode thioesterase proteins, specifically, the proteins of SEQ ID NOS: 7-8.

### EXAMPLE 4

### Identification of a Daptomycin NRPS

Identification of dptD as a Daptomycin NRPS Subunit

The predicted translation products of the dptD DNA sequences described above (Examples 2 and 3) were inspected visually for the occurrence of various protein motifs described in the NRPS literature. A dptD condensation (“M”) motif, indicative of a condensation domain, was identified at nucleotides 78486-78509 of SEQ ID NO: 1 (all of the nucleotide positions discussed in Examples 4-6 refer to SEQ ID NO: 1). See, e.g., Kleinkauf et al., Eur. J. Biochem., 236, pp. 335-351 (1996) for the various motifs in the NRPS; and Pospiech et al., Microbiology, 142, pp. 741-746 (1996). An ATP-binding (“C”) motif was identified at nucleotides 79896-79928, an ATP-binding (“E”) motif was identified at nucleotides 80451-80486, an ATPase (“F”) motif was identified at nucleotides 80556-80579, and an ATP-binding (“G”) motif was identified at nucleotides 80652-80675. These motifs collectively are indicative of an adenylation domain. A thiolation (“J”) motif, indicative of a thiolation (PCP) domain, was identified at nucleotides 81048-81062. The above motifs, and the domains that they signify, belong to module 1 of dptD; in terms of daptomycin synthetase, this is module 12.

Another dptD condensation (“M”) motif, indicative of a condensation domain, was identified at nucleotides 81621-81644. Another ATP-binding (“C”) motif was identified at nucleotides 83114-83147, an ATP-binding (“E”) motif was identified at nucleotides 83667-83702, an ATPase (“F”) motif was identified at nucleotides 83772-83795, and an ATP-binding (“G”) motif was identified at nucleotides 83868-83891. The above motifs collectively are indicative of another adenylation domain. Also a thiolation (“J”) motif, an indicator of a thiolation (PCP) domain, was identified at nucleotides 84255-84269. The above motifs, and the domains that they signify, belong to module 2 of dptD; in terms of daptomycin synthetase, this is module 13.

The DptD amino acid sequences corresponding to the above-described predicted motifs and domains were identified (all of the amino acid positions for DptD refer to the amino acid positions in SEQ ID NO: 7). The motifs, and the domains that they signify, belonging to module 1 of DptD (corresponding to module 12 of daptomycin synthetase) are as follows: A DptD condensation (“M”) motif was identified at coordinates 144-151; an ATP-binding (“C”) motif was identified at coordinates 614-624; an ATP-binding (“E”) motif was identified at coordinates 799-810; an ATPase (“F”) motif was identified at coordinates 834-841; an ATP-binding (“G”) motif was identified at coordinates 866-873; and a thiolation (“J”) motif was identified at coordinates 998-1002.

The DptD motifs, and the domains that they signify, belonging to module 2 of DptD (corresponding to module 13 of daptomycin synthetase) are as follows: A DptD condensation (“M”) motif was identified at coordinates 1189-1196; an ATP-binding (“C”) motif was identified at coordinates 1687-1697; an ATP-binding (“E”) motif was identified at coordinates 1871-1882; an ATPase (“F”) motif was identified at coordinates 1906-1913; an ATP-binding (“G”) motif was identified at coordinates 1938-1945; and a thiolation (“J”) motif was identified at coordinates 2067-2071. The ATP-binding motifs are representative of adenylation domains.

Identification of dptA and dptBC as Daptomycin NRPS Subunits

Certain M, C, E, F, G and J motifs were identified in a similar fashion in dptA and dptBC. The sequence and type of each motif, the genes and modules in which each motif is found, as well as the amino acid and nucleotide coordinates of each motif, are shown below in Table 1:

TABLE 1

| | | | | Amino | |
|---|---|---|---|---|---|
| | | Mo- | | Acid | |
| | Mod- | tif | | Coordi- | Nucleotide |
| Gene | ule | Type | Sequence | nates | Coordinates |
| dptA | 1 | M | HHIALDGY | 138-145 | 38966-38989 |
| dptA | 1 | C | QTSGSTGRPKG | 603-613 | 40361-40393 |
| dptA | 1 | E | GELYLAGEGLAR | 784-795 | 40904-40939 |
| dptA | 1 | F | RMYRTGDL | 819-826 | 41009-41032 |
| dptA | 1 | G | RIELGEVQ | 851-858 | 41105-41128 |
| dptA | 1 | J | LGGHS | 981-985 | 41495-41509 |
| dptA | 2 | M | HHTAGDGA | 1167-1174 | 42053-42076 |
| dptA | 2 | C | YTSGSTGRPKG | 1657-1667 | 43523-43555 |
| dptA | 2 | E | GELHVAGEGLAR | 1843-1854 | 44081-44116 |
| dptA | 2 | F | RMYRTGDL | 1878-1885 | 44186-44209 |
| dptA | 2 | G | RIELGEVE | 1910-1917 | 44282-44305 |
| dptA | 2 | J | LGGDS | 2041-2045 | 44675-44689 |
| dptA | 3 | M | HHVILDGW | 2751-2758 | 46805-46828 |
| dptA | 3 | C | YTSGSTGLPKG | 3238-3248 | 48266-48298 |
| dptA | 3 | E | GELYVAGDGLAR | 3420-3431 | 48812-48847 |
| dptA | 3 | F | RMYRTGDL | 3455-3462 | 48917-48940 |
| dptA | 3 | G | RIELGEVE | 3487-3494 | 49013-49036 |
| dptA | 3 | J | LGGHS | 3616-3620 | 49400-49414 |
| dptA | 4 | M | HHIAGDGW | 3806-3813 | 49970-49993 |
| dptA | 4 | C | YTSGSTGRPKG | 4292-4302 | 51428-51460 |
| dptA | 4 | E | GEMYVAGAGLAR | 4490-4501 | 52022-52057 |
| dptA | 4 | F | RLYRTGDL | 4525-4532 | 52127-52150 |
| dptA | 4 | G | RIELGEIE | 4557-4564 | 52223-52246 |
| dptA | 4 | J | LGGHS | 4688-4692 | 52616-52630 |
| dptA | 5 | M | HHIAGDGW | 4873-4880 | 53171-53194 |
| dptA | 5 | C | HTSGSTGRPKG | 5363-5373 | 54641-54673 |
| dptA | 5 | E | GEIHIAGSGLAR | 5553-5564 | 55211-55246 |
| dptA | 5 | F | RMYRTGDL | 5587-5594 | 55313-55336 |
| dptA | 5 | G | RIELGDVE | 5619-5626 | 55409-55432 |
| dptA | 5 | J | LGGDS | 5749-5753 | 55799-55813 |
| dptBC | 1 | M | HHVILDGW | 142-149 | 56467-56490 |
| dptBC | 1 | C | HTSGSTGRPKG | 611-621 | 57874-57906 |
| dptBC | 1 | E | GELYLAGTQLAR | 803-814 | 58450-58485 |
| dptBC | 1 | F | RMYRTGDL | 838-845 | 58555-58578 |
| dptBC | 1 | G | RIEPAEIE | 870-877 | 58651-58674 |
| dptBC | 1 | J | AGGHS | 998-1002 | 59035-59049 |
| dptBC | 2 | M | HHIAGDGW | 1184-1191 | 59593-59616 |
| dptBC | 2 | C | YTSGSTGRPKG | 1691-1701 | 61114-61146 |
| dptBC | 2 | E | GELYVAGVGLAR | 1873-1884 | 61660-61695 |
| dptBC | 2 | F | RMYRTGDL | 1908-1915 | 61765-61788 |
| dptBC | 2 | G | RVELGEVE | 1940-1947 | 61861-61884 |
| dptBC | 2 | J | LGGHS | 2069-2073 | 62248-62262 |
| dptBC | 3 | M | HHVAFDAM | 2259-2266 | 62818-62841 |
| dptBC | 3 | C | YTSGSTGRPKG | 2740-2750 | 64261-64293 |
| dptBC | 3 | E | GELYVAGVGLAR | 2923-2934 | 64810-64845 |
| dptBC | 3 | F | RMYRTGDL | 2958-2965 | 64915-64938 |
| dptBC | 3 | G | RVELGEVE | 2990-2997 | 65011-65034 |
| dptBC | 3 | J | LGGDS | 3118-3122 | 65395-65409 |
| dptBC | 4 | M | HHVVLDGW | 3805-3812 | 67456-67479 |
| dptBC | 4 | C | YTSGSTGRPKG | 4282-4292 | 68887-68919 |
| dptBC | 4 | E | GELYVAGVGLAR | 4464-4475 | 69433-69468 |
| dptBC | 4 | F | RMYRTGDL | 4499-4506 | 69538-69561 |
| dptBC | 4 | G | RVELGEVE | 4531-4538 | 69634-69657 |
| dptBC | 4 | J | LGGHS | 4662-4666 | 70027-70041 |
| dptBC | 5 | M | HHIAGDGW | 4852-4859 | 70597-70620 |
| dptBC | 5 | C | YTSGSTGQPKG | 5340-5350 | 72061-72093 |
| dptBC | 5 | E | GELYIAGDGLAR | 5526-5537 | 72619-72654 |
| dptBC | 5 | F | RMYRTGDL | 5561-5568 | 72724-72747 |
| dptBC | 5 | G | RVELGEVE | 5593-5600 | 72820-72843 |
| dptBC | 5 | J | LGGHS | 5722-5726 | 73206-73221 |
| dptBC | 6 | M | HHIAGDGW | 5913-5920 | 73780-73803 |
| dptBC | 6 | C | YTSGSTGRPKG | 6394-6404 | 75223-75255 |
| dptBC | 6 | E | GELYLAGAGLAR | 6584-6595 | 75793-75828 |
| dptBC | 6 | F | RMYRTGDL | 6619-6626 | 75898-75921 |
| dptBC | 6 | G | RVELGEVE | 6651-6658 | 75994-76017 |
| dptBC | 6 | J | LGGDS | 6781-6785 | 76384-76398 |

The amino acid coordinates in Table 1 refer to the amino acid sequence of each protein (DptA: SEQ ID NO: 9; DptBC: SEQ ID NO: 11). The nucleotide position refers to the nucleotide position in SEQ ID NO: 1.

### EXAMPLE 5

### Amino Acid Pocket Code Annotation

The amino acid pocket code refers to a set of amino acid residues in the adenylation (A) domain that are believed to be involved in recognition and or binding of the cognate amino acid. The amino acid pocket code for the thirteen daptomycin synthetase modules are shown below (Table 2).

The amino acid pocket code for the daptomycin synthetase modules was identified by Blast analysis or visual inspection of alignments created using MacVector 7.0 of the putative Dpt translation product aligned with NRPS A domains (amino acid binding pockets) as described in Stachelhaus et al. (1999), The specificity-conferring code of adenylation domains in nonribosomal peptide synthetases, Chem. Biol., 6:493-505. See also Challis et al., (2000), Predictive, structure-based model of amino acid recognition by nonribosomal peptide synthetase adenylation domains, Chem. Biol. 7:211-224.

The amino acid coordinates in Table 2 refer to the amino acid sequence of each protein (DptA: SEQ ID NO: 9; DptBC: SEQ ID NO: 11; DptD: SEQ ID NO: 7). The nucleotide position refers to the nucleotide position in SEQ ID NO: 1.

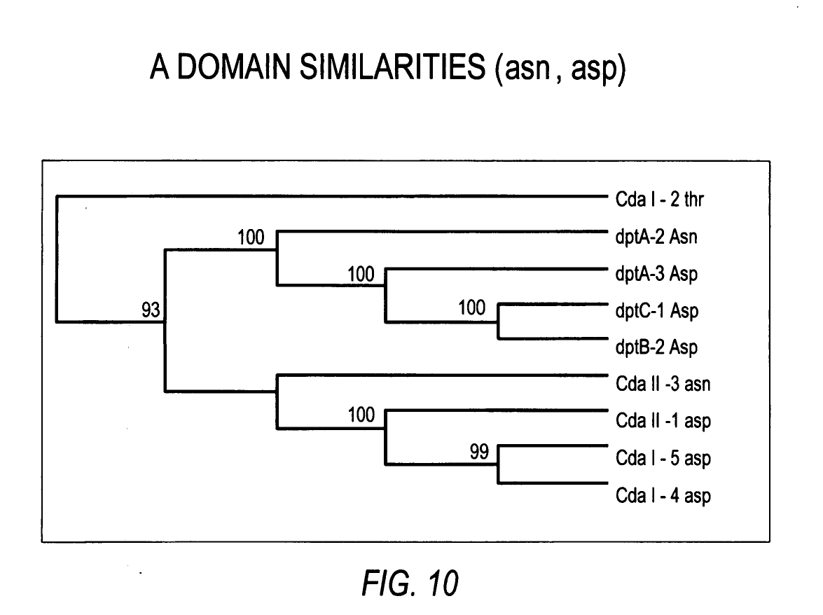

Similarities between essentially the entire adenylation domains for aspartate and asparagine in the daptomycin gene cluster and for the adenylation domains for aspartate, asparagine and threonine in the CDA III NRPS of Streptomyces coelicolor are shown in FIG. 10. Amino acids were aligned and the dendrogram was constructed using the MacVector. The nomenclature is as follows: the name of the gene—the module number in the gene—the amino acid activated (one letter code). The alignment shows that the adenylation domains for aspartate and asparagine in the daptomycin gene cluster are more similar to each other than they are to a domain from an unrelated amino acid such as threonine. Further, the alignment shows that the adenylation domains for aspartate and asparagine in the daptomycin gene cluster are more similar to each other than they are similar to the modules for aspartate and asparagine in CDA.

### EXAMPLE 6

### Identification of Epimerase Domains in Daptomycin NRPS

The amino acid sequences of DptA, DptBC and DptD were inspected for sequences that are characteristic of epimerase domains. Epimerase domains are responsible for converting an L-amino acid to a D-amino acid and are typically encoded by approximately 1.4-1.6 kb of DNA.

It was expected that there would be a total of two epimerase domains in the daptomycin gene cluster, because it was known that daptomycin contained two D-amino acids, D-Ala and D-Ser. One epimerase domain was identified in each of module 8 (D-Ala) and module 11 (D-Ser). Module 8 and 11 are approximately 1.4 kb larger than modules that did not contain an epimerase domain (approximately 4.6 kb each for modules 8 and 11 compared to 3.2 kb each for modules not containing an epimerase domain). Further, modules 8 and 11 contain motifs that are indicative of an epimerase domain, including the motifs K, L, M, N, O, P and Q (see Kleinkauf and Von Dohren, 236:355-351 (1996)). See Table 3.

Surprisingly, an epimerase domain was also identified in module 2. Module 2 is 1.6 kb larger than expected. Further, module 2 contains a number of motifs that are characteristic of an epimerase domain, including motifs K, L, M, N, O, P and Q. See Table 3. This unexpected finding suggests that the asparagine in daptomycin is in the D configuration.

TABLE 3

| | | Mo- | | | |
|---|---|---|---|---|---|
| | Mod- | tif | | Amino Acid | Nucleotide |
| Gene | ule | Type | Sequence | Coordinates | Coordinates |
| dptA | 2 | K | RWPVVEWL | 2100-2107 | 44852-44875 |
| dptA | 2 | L | VRERHDAW | 2146-2153 | 44990-45013 |
| dptA | 2 | M | HHLVVDGVSWR | 2237-2251 | 45263-45307 |
| | | | IVLG | | |
| dptA | 2 | N | VVDVEGHGRN | 2374-2383 | 45674-45703 |
| dptA | 2 | O | TVGWFTSIYPV | 2395-2407 | 45737-45775 |
| | | | RL | | |
| dptA | 2 | P | PDQGLGY | 2439-2445 | 45869-45689 |
| dptA | 2 | Q | FGFNYLG | 2467-2473 | 45953-45973 |
| dptBC | 3 | K | RWPVVEWL | 3183-3190 | 65590-65613 |
| dptBC | 3 | L | VRDRHEAW | 3229-3236 | 65728-65751 |
| dptBC | 3 | M | HHLVVDGVSWR | 33315-3329 | 65986-66030 |
| | | | VVLG | | |
| dptBC | 3 | N | VVDVEGHGRN | 3452-3461 | 66397-66426 |
| dptBC | 3 | O | TVGWFTSVYPV | 3473-3485 | 66460-66498 |
| | | | RV | | |
| dptBC | 3 | P | PDQGLGY | 3517-3523 | 66592-66612 |
| dptBC | 3 | Q | FGFNYLG | 3545-3551 | 66676-66696 |
| dptBC | 6 | K | RWPVVEWL | 6846-6853 | 76579-76602 |
| dptBC | 6 | L | VRDRHEAW | 6892-6899 | 76717-76740 |
| dptBC | 6 | M | HHLVVDGVSWR | 6978-6992 | 76975-77019 |
| | | | VVLG | | |
| dptBC | 6 | N | VVDVEGHGRN | 7115-7124 | 77383-77415 |
| dptBC | 6 | O | TVGWFTSVYPV | 7136-7148 | 77449-77487 |
| | | | RV | | |
| dptBC | 6 | P | PDQGLGY | 7180-7186 | 77581-77601 |
| dptBC | 6 | Q | FGFNYLG | 7208-7214 | 77665-77685 |

The amino acid coordinates in Table 3 refer to the amino acid sequence of each protein (DptA: SEQ ID NO: 9; DptBC: SEQ ID NO: 11; DptD: SEQ ID NO: 7). The nucleotide position refers to the nucleotide position in SEQ ID NO: 1.

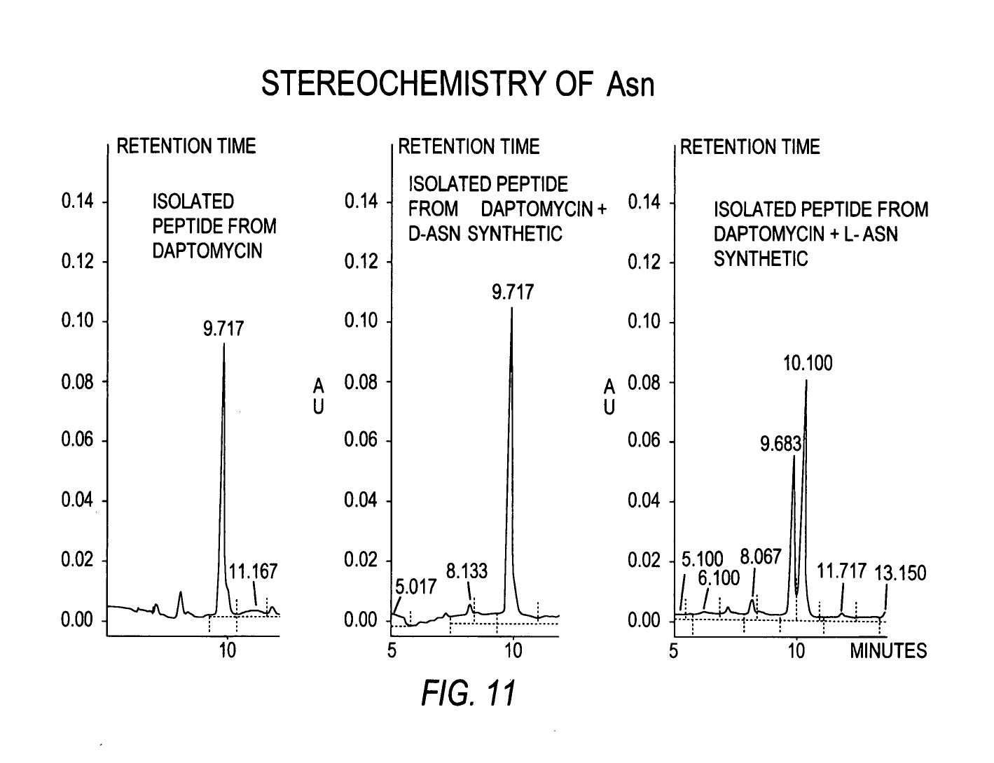

To confirm that the asparagine in daptomycin was in the D configuration, high pressure liquid chromatography (HPLC) was performed. A hexa-peptide containing the amino acids ornithine, glycine, threonine, aspartic acid, asparagine, and deacylated tryptophan (Trp-Asn-Asp-Orn-Gly-Thr) (SEQ ID NO: 168) was isolated from daptomycin by degradation. The peptide above was analyzed by HPLC under conditions that would separate the peptide containing either the D-Asn or L-Asn. The HPLC showed only a single large peak for the isolated peptide above. See FIG. 11, left panel. The peptide isolated from daptomycin was mixed with a peptide of the same sequence that had been synthesized in the laboratory and which contained D-Asn. The peptide mixture was analyzed by HPLC under the same conditions as before and shown to contain only a single peak. See FIG. 11, middle panel. In addition, the peptide isolated from daptomycin was mixed with a synthetic peptide of the same sequence that contained L-Asn. HPLC analysis displayed two peaks. See FIG. 11, right panel. These experiments confirm that naturally-occurring daptomycin contains D-Asn, not L-Asn.

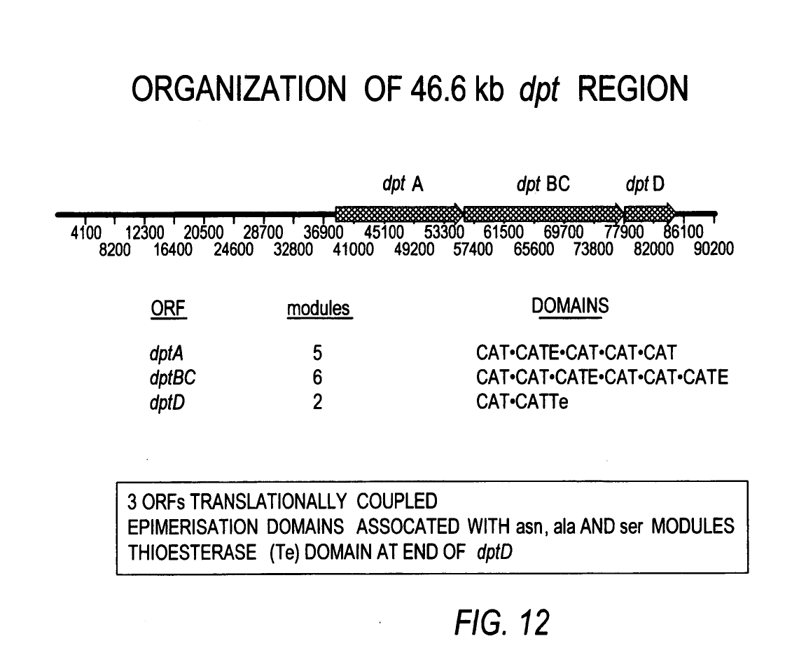

From the experiments presented in Examples 2-7, the organization of the daptomycin NRPS was determined. FIG. 12 shows the organization of dptA, dptBC, and dptD. dptA contains five modules (modules 1-5), dptB contains 6 modules (modules 6-11), and dptD contains two modules (modules 12-13) and a thioesterase domain. Table 4 summarizes the correspondence between the 13 modules, their domains, the dpt genes, and their cognate amino acids. “C” represents a catalytic domain, “A” represents an adenylation domain, “T” represents a thiolation domain, “E” represents an epimerase domain, and “Te” represents a thioesterase domain.

TABLE 4

| | Cognate | | |
|---|---|---|---|
| | Amino | | |
| Module | Acid | Domains | Gene |
| 01 | L-Trp | CAT | dptA |
| 02 | D-Asn | CATE | dptA |
| 03 | L-Asp | CAT | dptA |
| 04 | L-Thr | CAT | dptA |
| 05 | Gly | CAT | dptA |
| 06 | L-Orn | CAT | dptBC |
| 07 | L-Asp | CAT | dptBC |
| 08 | D-Ala | CATE | dptBC |
| 09 | L-Asp | CAT | dptBC |
| 10 | Gly | CAT | dptBC |
| 11 | D-Ser | CATE | dptBC |
| 12 | L-MG | CAT | dptD |
| 13 | Kyn | CAT-Te | dptD |

### EXAMPLE 7

### Transformation of Streptomyces lividans with the Daptomycin Gene Cluster from Streptomyces roseosporus

E. coli cells containing the BAC DNA from clone B12:03A05 (see Example 2) were grown in 5 mL of Luria Broth (LB; Difco) with agitation (250 rpm) overnight at 37° C. The BAC DNA was isolated by a standard alkaline lysis procedure (see Sambrook et al., supra, “Small scale preparation of plasmid DNA”).

S. lividans TK64 spores were used to inoculate 25 mL of YEME+sucrose media and the culture was incubated for 40 hours at 30° C. The cultures were then harvested and the mycelium was pelleted away from the supernatant and washed several times with P-buffer (Practical Streptomyces Genetics; Tobias Kieser, Mervyn J. Bibb, Mark J. Buttner, Keith F. Chater and David Hopwood (John Innes Foundation, Norwich, 2000) (“Practical Streptomyces Genetics”). Fresh protoplasts were prepared according to the method described in Practical Streptomyces Genetics, supra (p. 56) and aliquoted into 0.5 mL portions (approximately 10⁸-10⁹ protoplasts) and pelleted by centrifugation at 3000 rpm for 7 minutes. Most of the supernatant was removed, leaving the pellet and approximately 50 μL of the supernatant. The pellet was resuspended in the remaining supernatant, to which was added 5 μL of BAC DNA from clone B12:03A05 (50 ng/μL in TE). This suspension was gently mixed before and after adding 350 μL of a 25% PEG-1000 in P-buffer solution (Practical Streptomyces Genetics, supra).

The protoplast suspension mixture was spread, in equal amounts, onto three dried R5T plates (dried to lose approximately 15% of their original weight; see Practical Streptomyces Genetics, supra). Inoculated plates were incubated overnight at 30° C. After 16-18 hours of growth, the plates were overlaid with 3 mL of an apramycin solution (1 mg/mL) in 20% glycerol to provide a final concentration of approximately 100 μg/mL on each plate, and the plates incubated at 30° C. After three days, the plates were determined, by examination, to contain colonies which were growing in the presence of the apramycin selection. Two colonies were picked and streaked onto two F10A agar plates (2.5% agar, 0.3% calcium carbonate, 0.5% distillers solubles, 2.5% soluble starch, 0.5% yeast extract, 0.2% dextrose and 0.5% bactopeptone; suspended in 1 L deionized and autoclaved water) containing 100 μL/mL of apramycin and allowed to incubate at 30° C. until the colonies sporulated. Spores were harvested according to the methods described in Practical Streptomyces Genetics, supra and stored as 20% glycerol suspensions at −20° C.

The spores derived from the transformation of S. lividans with BAC DNA containing the daptomycin gene cluster (from clone B12:03A05, CBUK136742) were grown in an appropriate medium and analyzed by high pressure liquid chromatography (HPLC) and LC-MS to determine if they produced a wild-type lipopeptide profile (see Example 9).

### EXAMPLE 8

### Fermentation of Streptomyces lividans TK64 Clone Containing the Daptomycin Gene Cluster

Spores of the Streptomyces lividans TK64 clone containing the daptomycin gene cluster (from clone B12:03A05) were harvested by suspending a 10 day old slant culture of medium A (2% irradiated oats (Quaker), 0.7% tryptone (Difco), 0.2% soya peptone (Sigma), 0.5% sodium chloride (BDH), 0.1% trace salts solution, 1.8% agar no. 2 (Lab M), 0.01% apramycin (Sigma)) in 5 mL 10% aqueous glycerol (BDH)). 1 mL of this suspension, in a 1.5 mL cryovial, comprises the starting material, which was retrieved from storage at −135° C. A pre-culture was produced by aseptically placing 0.3 mL of the starting material onto a slope of medium A1 and incubating for 9 days at 28° C.

A seed culture was generated by aseptically treating the pre-culture with 4 mL of a 0.1% Tween 80 (Sigma) solution and gently macerating the slope surface to generate a suspension of vegetative mycelium and spores. A two mL aliquot of this suspension was transferred into a 250 mL baffled flask containing 40 mL of nutrient solution S (1% D-glucose (BDH), 1.5% glycerol (BDH), 1.5% soya peptone (Sigma), 0.3% sodium chloride (BDH), 0.5% malt extract (Oxoid), 0.5% yeast extract (Lab M), 0.1% Junlon PW100 (Honeywell and Stein Ltd), 0.1% Tween 80 (Sigma), 4.6% MOPS (Sigma) adjusted to pH 7.0 and autoclaved)) and shaken at 240 rpm for 44 hours at 30° C.

Production cultures were generated by aseptically transferring 5% of the seed culture to baffled 250 mL flasks containing 50 mL medium P (1% glucose (BDH), 2% soluble starch (Sigma), 0.5% yeast extract (Difco), 0.5% casein (Sigma), 4.6% MOPS (Sigma) adjusted to pH 7 and autoclaved)) and shaken at 240 rpm for up to 7 days at 30° C.

### EXAMPLE 9

### Purification and Analysis of the A21978C Lipopeptides from Fermentations of the Streptomyces lividans TK64 Clone Containing the Daptomycin Gene Cluster

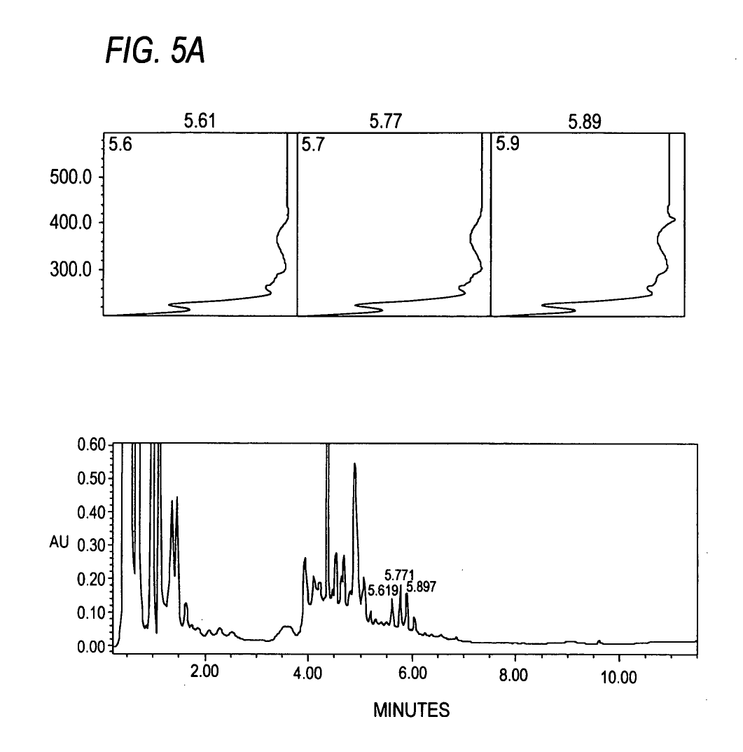

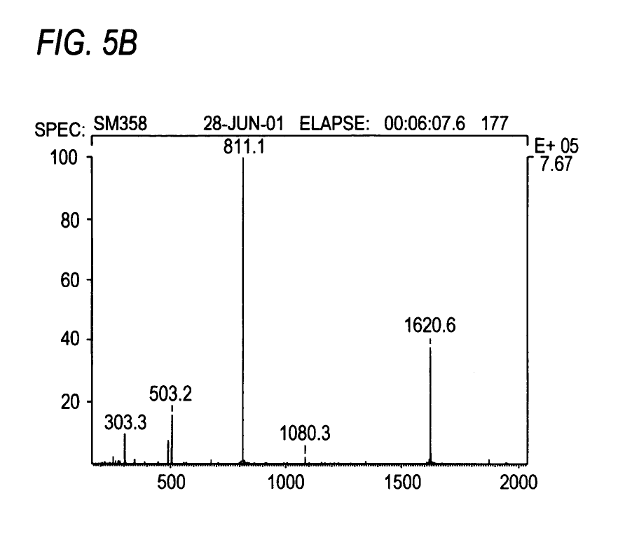

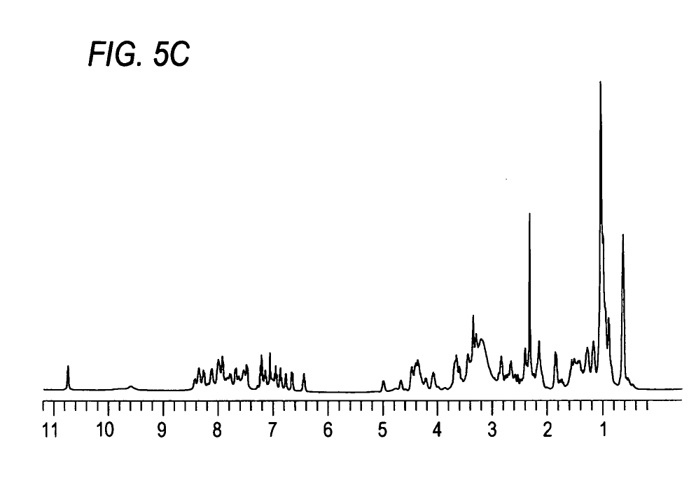

Production cultures described in Example 8 were sampled for analysis by aseptically removing 2 mL of the whole culture and centrifuging for 10 minutes prior to analysis. Volumes up to 50 microlitres of the supernatant were analyzed to monitor for production of the native lipopeptides (A21978C) as produced by Streptomyces roseosporus. This analysis was performed at ambient temperature using a Waters Alliance 2690 HPLC system and a 996 PDA detector with a 4.6×50 mm Symmetry C8 3.5 μm column and a Phenomenex Security Guard C8 cartridge. The gradient initially holds at 90% water and 10% acetonitrile for 2.5 minutes, followed by a linear gradient over 6 minutes to 100% acetonitrile. The flow rate is 1.5 mL per minute and the gradient is buffered with 0.01% trifluoroacetic acid. By day 2 of the fermentation, production of three of the native lipopeptides, C1, C2 and C3, with UV/visible spectra identical to that of daptomycin, was evident, as shown by HPLC peaks with retention times of 5.62, 5.77 and 5.90 minutes (λmax 223.8, 261.5 and 364.5 nm) under the analytical conditions stated, as shown in FIG. 5A. The lipopeptides then remained evident in the fermentation at each sample point during the 7-day period. Total yields of lipopeptides C1, C2 and C3 ranged from 10-20 mg per liter of fermentation material.

Liquid chromatography-mass spectrometry (LC-MS) analysis was performed on a Finnigan SSQ710c LC-MS system using electrospray ionization in positive ion mode, with a scan range of 200-2000 daltons and 2 second scans. Chromatographic separation was achieved on a Waters Symmetry C8 column (2.1×50 mm, 3.5 μm particle size) eluted with a linear water-acetonitrile gradient containing 0.01% formic acid, increasing from 10% to 100% acetonitrile over a period of six minutes after a initial delay of 0.5 minutes, then remaining at 100% acetonitrile for a further 3.5 minutes before re-equilibration. The flow rate was 0.35 mL/minute and the method was run at ambient temperature.

The identification of the three native lipopeptides was confirmed, as indicated by molecular ions ([M+H]⁺) at m/z of 1634.7, 1648.7 and 1662.7, which is in agreement with the masses reported for the major A21978C lipopeptide metabolites C1, C2 and C3, respectively, produced by Streptomyces roseosporus (Debono et al., J. Antibiotics, 40, pp. 761-777 (1987)).

Similar experiments were performed using the BAC clones 01G06, B12:06A12, B12:12F06 and B12:18H04. None of the S. lividans cells containing any one of these BAC clones was able to produce daptomycin.

### EXAMPLE 10

### Fed-Batch Fermentation of Streptomyces lividans TK64 Clone Containing the Daptomycin Gene Cluster for the Production of Daptomycin

Cells of the Streptomyces lividans TK64 clone containing the daptomycin gene cluster (from clone B12:03A05) were regenerated by suspending a 10 day old slope culture of medium A (see Practical Streptomyces Genetics; 2% irradiate oats (Quaker), 0.7% tryptone (Difco), 0.2% soya peptone (Sigma), 0.5% sodium chloride (BDH), 0.1% trace salts solution, 1.8% agar no. 2 (Lab M), 0.01% apramycin (Sigma) in 5 mL 10% aqueous glycerol (BDH)). A 1.5 mL cryovial containing 1 mL of starting material was retrieved from storage at −135° C. and thawed rapidly. A pre-culture was produced by aseptically placing 0.3 mL of the starting material onto a slope of medium A and incubating for 9 days at 28° C. Material for inoculation of the seed culture was generated by aseptically treating the preculture with 4 mL of a 0.1% Tween 80 (Sigma) solution and gently macerating the slope surface to generate a suspension of vegetative mycelium and spores.

A seed culture was produced by aseptically placing 1 mL of the inoculation material into a 2 L baffled Erlenmeyer flask containing 250 mL of nutrient solution S (see Practical Streptomyces Genetics, supra) shaken at 240 rpm for 2 days at 30° C.

A production culture was generated by aseptically transferring the seed culture to a 20 L fermenter containing 14 liters of nutrient solution P (see Practical Streptomyces Genetics, supra). The production fermenter was stirred at 350 rpm, aerated at 0.5 vvm, and temperature controlled at 30° C. After 20 hours incubation a 50% (w/v) glucose solution was fed to the culture at 5 g/hr throughout the fermentation.

After 40 hours incubation, a 50:50 (w/w) blend of decanoic acid:methyl oleate (Sigma and Acros Organics, respectively) was fed to the fermenter at 0.5 g/hr for the remainder of fermentation. The culture was harvested after 112 hours, and the biomass removed from the culture supernatant by batch processing through a bowl centrifuge.

The biomass was discarded and the clarified fermentation broth was retained for extraction. The broth (approximately 10 L) was loaded onto a 60 mm (diameter) by 300 mm (length) column of HP20 resin, which had been pre-equilibrated with water, at a rate of 100 mL/min. The column was washed with 2 L of water and then with 1.5 L of 80% methanol (in water) at a similar flow rate. Finally, the bound material was eluted with 2 L methanol and then taken to an aqueous concentrate under vacuum. The concentrate was diluted to 1 L with purified water and partitioned with ethyl acetate (700 mL) three times. The ethyl acetate fraction was analyzed and discarded, and the aqueous layer was lyophilized to a powder.

Daptomycin was isolated by high performance liquid chromatography (HPLC) using a radially compressed cartridge column consisting of two 40×100 mm Waters Nova-Pak C18 6 μm units and a 40×10 mm Guard-Pak with identical packing. Lyophilized material (150 to 200 mg) was dissolved in water and chromatographed on the columns using a gradient in which the initial conditions were 90% water and 10% acetonitrile, followed by a linear increase over 10 minutes to 20% water and 80% acetonitrile, and then immediately ramping up to 100% acetonitrile over a further minute. UV absorption at 223 nm was monitored for elution of daptomycin. The daptomycin peak eluted at about 9 minutes and was collected and combined over many repeated runs. The sample was then evaporated under vacuum and then dried in vacuo to yield 30 mg of purified compound. Only a proportion of the total material was processed.

The purified compound was first analyzed by reversed phase HPLC at ambient temperature on a 4.6×50 mm Waters Symmetry C8 3.5 μm particle size column with a Phenomenex Security Guard C8 cartridge using a Waters Alliance 2690 HPLC system and a 996 PDA detector. The column was eluted with a water-acetonitrile gradient, initially holding at 90% water for 2.5 minutes and then rising linearly over 6 minutes to 100% acetonitrile, at a flow rate of 1.5 mL/minute. The gradient was buffered with 0.01% trifluoroacetic acid. This chromatographic analysis confirmed that the retention time (5.52 mins) and the UV absorption spectrum (λₘₐₓ 223.8, 261.5, 366.9 nm) of the purified compound matched those of daptomycin. LC-MS (ESI) confirmed the molecular ion MH⁺ as 1620.6 (FIG. 5B) and the ¹H NMR (D6-DMSO) gave a good visual match with that recorded for daptomycin (FIG. 5C).

The identification of the material as daptomycin was further confirmed by ¹³CNMR experiments.

Feed-batch fermentation may also be accomplished at a larger scale, for example at 60,000 liters.

### EXAMPLE 11

### The Use of Daptomycin Genes for Yield Enhancement

Chapter 1 Duplication of a Positive Regulatory Gene

A neutral genomic site in the chromosome of Streptomyces roseosporus is identified by transposon mutagenesis with TN5097, or a related transposon, followed by fermentation analysis. The neutral site is excised from the chromosome using a restriction endonuclease that cuts outside of the neutral site and transposon, and cloned in Escherichia coli, selecting for the expression of the antibiotic resistance marker in the transposon (hygromycin resistance in the case of TN5097). An example of this approach was used to identify a neutral site in Streptomyces fradiae, the tylosin producer. See Baltz et al., Antonie van Leeuwenhoek, 71, pp. 179-187 (1997), incorporated herein by reference in its entirety. An example of identifying a neutral site in S. roseosporus is described in McHenney et al., J. Bacteriol., 180, pp. 143-151 (1998), incorporated herein by reference in its entirety.

The regulatory gene from the daptomycin gene cluster (SEQ ID NO: 109) is cloned into a plasmid within the neutral site. A suitable plasmid would be one containing an antibiotic resistance gene for the selection of primary recombinants containing single crossovers, a counter-selectable marker such as the wild type rpsL gene, a ribosomal protein gene that confers sensitivity to streptomycin (Hosted and Baltz, J. Bacteriol., 179, pp. 180-186 (1997)) for selection of recombinants containing double crossovers that insert the cloned regulatory gene, and upstream and downstream sequences, into the chromosomal neutral site, and eliminate the plasmid sequences, and a thermal sensitive replicon that would facilitate the curing of the plasmid. The double crossover is done in a host strain that is normally resistant to streptomycin because it contains a mutation in the rpsL gene. Since the wild type (streptomycin-sensitive) allele of rpsL is dominant over streptomycin resistance, recombinants expressing streptomycin resistance must have eliminated the rpsL gene on the plasmid by a double crossover in the two arms of the neutral site, thus inserting the cloned daptomycin regulatory gene into the chromosome. Recombinants are fermented to verify that they produce an increased yield compared to the parental strain lacking the cloned daptomycin regulatory gene.

Duplication of ABC Transporter Genes

One or more of the ABC transporter genes from the daptomycin gene cluster, including upstream and downstream sequences, are cloned into the neutral site vector described above and inserted by double crossover into the S. roseosporus chromosome as described in Example 11A. Recombinants are fermented to verify that they produce increased levels of daptomycin compared to the parental strain lacking the cloned ABC transporter genes.

Duplication of novA, B, C Homologs

The segment of DNA containing the novA, B, C homology from the daptomycin gene cluster, including the upstream and downstream sequences, is cloned into the neutral site vector and inserted by double crossover into the S. roseosporus chromosome as described in Example 11A. Recombinants are fermented to verify that they produce increased levels of daptomycin compared to the parental strain lacking the cloned novA, B, C genes.

Duplication of Daptomycin Biosynthetic Genes

The daptomycin biosynthetic genes, dptA, dptBC, dptD, dptE, dptF, dptG and dptH, including the fatty acyl-CoA ligase, the three subunits of the NRPS, the integral thioesterase of dptD and the free thioesterase of dptH, are cloned into a BAC vector that contains the phiC31 attachment and integration functions (att/int) and oriT from plasmid RK2 (Baltz, Trends in Microbiol., 6: 76-83 (1998), incorporated herein by reference in its entirety) for conjugation from E. coli to S. roseosporus. The BAC containing the daptomycin genes is introduced into S. roseosporus by conjugation from E. coli S17.1, or a strain containing a self-replicating plasmid RK2 (Id.). Alternatively, the BAC vector inserts into the chromosome by homologous recombination into the daptomycin gene cluster. Recombinants are fermented to verify that they produce increased levels of daptomycin compared to the parental strain lacking the cloned daptomycin genes.

Duplication of Daptomycin Thioesterase Genes

The daptomycin gene cluster (SEQ ID NO: 1) contains at least two genes (dptD and dptH) having open reading frames (SEQ ID NO: 3 and SEQ ID NO: 6, respectively) or domains thereof that encode amino acid sequences which include conserved sequence motifs characteristic of proteins having thioesterase activity. See SEQ ID NO:7 and SEQ ID NO:8 for DptD and DptH amino acid sequences, respectively. Either one (or both) of these thioesterase genes or the thioesterase domains thereof can be duplicated by following the procedure of Example 11A, above.

A segment of DNA containing the dptD ORF sequences (e.g., SEQ ID NO: 1; SEQ ID NO: 3) optionally linked in an operative fashion to an expression control sequence (such as the natural one in SEQ ID NO: 1 or 2) and optionally including the upstream and downstream sequences, is cloned into a neutral site vector and inserted by double crossover into the S. roseosporus chromosome as described in Example 11A. Recombinants are fermented to verify that they produce increased levels of daptomycin compared to the parental strain lacking the cloned dptD gene.

Similarly, a segment of DNA containing the dptH ORF sequences (e.g., SEQ ID NO: 4, SEQ ID NO: 6) optionally linked in an operative fashion to an expression control sequence (such as the natural one in SEQ ID NOS: 1, 4 or 5) and optionally including the upstream and downstream sequences, is cloned into a neutral site vector and inserted by double crossover into the S. roseosporus chromosome as described in Example 11A. Recombinants are fermented to verify that they produce increased levels of daptomycin compared to the parental strain lacking the cloned dptH gene.

Other suitable hosts (i.e., those having NRPS or PKS multienzyme complexes) may be transformed with segments of DNA encoding proteins from the daptomycin gene cluster having thioesterase activity for improved peptide production. Alternatively, polypeptides encoded by such segments of DNA may be introduced into S. roseosporus or said other suitable hosts by protein transfer techniques well-known to those of skill in the art.

Duplication of Daptomycin Resistance Genes

The daptomycin resistance gene(s) are identified by cloning and expression in an appropriate streptomycete host that is naturally susceptible to daptomycin. The cloned daptomycin resistance gene(s) are inserted into the neutral site vector within the neutral site, and inserted into the S. roseosporus chromosome by double crossover as described in Example 11A. Recombinants are fermented to verify that they produce increased levels of daptomycin compared to the parental strain lacking the cloned daptomycin resistance genes.

Duplication of Daptomycin Biosynthetic Genes and Accessory Genes

The BAC clone B12:03A05 was introduced into wild-type Streptomyces roseosporus A21978.6 (ATCC Deposit No. 31568, CBUK 136737) and Streptomyces roseosporus A21978.65 (NRRL Deposit No. 15998, CBUK 136879) by conjugation as described in Practical Streptomyces Genetics, supra, to create exconjugants, CBUK 136927 and CBUK 138016, respectively. The parent and the exconjugant strains were fermented as described in Example 8 in triplicate in medium P to which 0.1% each of filter sterilized glutamic acid, Na salt (BDH) ornithine (Sigma) and aspartic acid (Sigma) were added.

Cultures were sampled and analyzed by HPLC during a 10 day experiment. The HPLC was performed as described in Example 9 except that only 5 μL of the supernatant was injected. By 40-56 hours after commencing fermentation, production of three of the S. roseosporus native lipopeptides (A21978C1, A21978C2 and A21978C3) was clearly evident. These lipopeptides were present in the fermentation at every subsequent sample point, with diversification to other A21978C factors evident at the end of the time course. Averaged over the three fermentation replicates, the maximum yields of A21978C lipopeptides produced by the exconjugates CBUK 136927 (284 mg/L) and CBUK138016 (1488 mg/L) were approximately twice that produced by the parent strains CBUK 136737 (143 mg/L) and CBUK 136879 (726 mg/L), respectively.

### EXAMPLE 12

### The Use of Daptomycin Biosynthetic Genes to Produce Novel Products

A. Modification of the Peptide Structure by Site-Directed Mutagenesis of an Amino Acid Specificity Code: Conversion of Position 2 D-Asn to D-Asp.

The amino acid specificity codes for the thirteen amino acids in daptomycin are shown in Table 1 (see Example 6, above). See also Stachelhaus et al., Chem. Biol., 6, pp. 493-505 (1999), incorporated herein by reference in its entirety, for a discussion of identifying and altering adenylation domain amino acid specificity codes in NRPSs. The code for all three L-asp residues in positions 3, 7, and 9 of daptomycin are identical: DLTKLGAV (SEQ ID NO: 169) (where the letters indicate standard amino acid abbreviations). The code for D-Asn in position 2 is DLTKLGDV (SEQ ID NO: 170), and it differs by a single amino acid (a D instead of A in position 7). The D-Asn specificity code is changed to that specifying D-Asp by making a site specific change in the adenylation domain of module 2 in PS I.

The mutant version of module 2 is inserted into the S. roseosporus chromosome by gene replacement (see Example 11). A counter selectable marker (e.g., the wild type rpsL gene) is inserted into the adenylation domain of module 2 by gene replacement. The mutant module 2 adenylation domain containing the coding sequence for D-Asp, and containing flanking DNA (about 1 to 5 kb on each side of the specificity code) on an appropriate thermal sensitive plasmid is introduced into the S. roseosporus strain disrupted for daptomycin biosynthesis. Recombinants containing single crossovers are selected at the non-permissive temperature by selection for an antibiotic resistance marker on the plasmid (e.g., hygromycin, apramycin or thiostrepton resistance). If the host strain is streptomycin resistant by a mutation in the chromosomal rpsL gene, then the second crossover completing the gene replacement can be selected for streptomycin resistance. The recombinant is screened for antibiotic production. The novel derivative of daptomycin is separated and analyzed to confirm the structure according to methods described, e.g., in U.S. Pat. Nos. RE 32,333, RE 32,455, 4,874,843, 4,482,487, 4,537,717, and 5,912,226.

B. Molecular Exchange of an Amino Acid Coding Module for One of Different Amino Acid Specificity.

Daptomycin has four acidic amino acids: three L-asp residues at positions 3, 7, and 9, and a 3-methyl-Glu (3-MG) at position 12 (see Table 1, Example 6). Novel derivatives of daptomycin are generated by exchanging the adenylation domain that specifies 3-MG for one that specifies L-asp. The adenylation domain of the 3-MG module is inserted into segments of the L-asp module flanking the L-asp adenylation domain which has been removed by molecular genetic procedures. The hybrid 3-MG module containing the flanking DNA from an L-asp module is inserted into an appropriately constructed gene replacement vector, and the hybrid module is exchanged for an L-asp module by homologous double crossover as in Example 11A. This same procedure is repeated for the other two L-asp modules. The recombinants produce three novel derivatives of daptomycin containing 3-MG substituted for L-asp in positions 3, 7, or 9, and maintain the overall four negative charges in the molecule.

C. Exchange of a Non-Ribosomal Peptide Synthetase (NRPS) Subunit for One that Catalyzes the Incorporation of Different Amino Acid(s).

The gene that encodes the third subunit of the daptomycin NRPS (see Table 1, Example 6) contains two modules that encode the specificity for incorporation of amino acids 12 (3-MG) and 13 (L-kyn). The gene that encodes the third subunit for the biosynthesis of the cyclic lipopeptide CDA (Kempter et al., Angew. Chem. Int. Ed. Engl., 36, pp. 498-501 (1997); Chong et al., Microbiology, 144, pp. 193-199 (1998); each of which is incorporated by reference herein in its entirety) also encodes the last two amino acids, in this case amino acids 10 (3-MG) and 11 (L-trp). A derivative of daptomycin containing L-trp instead of L-kyn in position 13 is generated by disrupting gene dptD, and by replacing it with the gene that encodes PSIII for CDA. Expression of the PSIII gene from a strong promoter (e.g., the ermEp* promoter; Baltz, Trends Microbiol., 6, pp. 76-83 (1998), incorporated herein by reference in its entirety), and inserted into a neutral site in the S. roseosporus genome as described in Example 11A, allows CDA PSIII to complement the dptD mutation and results in the production of the altered daptomycin with L-trp replacing L-kyn. The recombinant is fermented and the product(s) of the recombinant are analyzed by LC-MS as described in Example 9.

Similar manipulations can be performed for trans-complementation for other subunits, i.e. to generate a disruption or deletion in a subunit of the daptomycin biosynthetic gene cluster, and then complement in trans by one or more natural or modified subunits from an NRPS (the latter can include trans-complementation by modified versions of daptomycin biosynthetic gene cluster subunits, such as can be generated using methods described throughout example 12, especially examples 12A, 12B or 12H, 12J). Trans-complementation between the NRPS subunits then leads to production of a novel nonribosomal peptide which can be analysed for as described in previous examples.

To perform a trans-complementation experiment using portions of the daptomycin biosynthetic gene cluster and the calcium dependent antibiotic (CDA) biosynthetic gene cluster, the set of daptomycin biosynthetic genes, or the set of daptomycin biosynthetic genes and accessory genes, such as those contained on the BAC clone B12:03A05 are introduced by transformation or conjugation into other natural or engineered strains or species of actinomycetes. The recipients may be known producers of secondary metabolites or uncharacterized strains, or may be generated by recombinant techniques to carry biosynthetic pathways other than that for biosynthesis of daptomycin. The transformants or ex-conjugants are fermented in a variety of media and whole broth or extracts thereof are screened for either novel daptomycin-like compounds or biological activity against daptomycin-resistant tester organisms.

In some instances, complementation is faciliated by inactivation of some of the genes in the daptomycin biosynthetic pathway. Sequences encoding a subunit of the NRPS in the BAC B12:03A05 can be deleted or replaced by a marker gene to form a modified B12:03A05 before introduction into a heterologous host that already expresses one or more native or introduced NRPS gene clusters. Enzyme subunits encoded by the modified B12:03A05 clone and by endogenous host NRPS genes may then associate in the cytoplasm to form a heteromeric multienzyme complex that leads to production of a novel peptide. In other cases, a deletion may be created after B12:03A05 or a portion comprising the daptomycin NRPS is introduced into a heterologous host that already expresses one or more native or introduced NRPS gene clusters. For example, a dptD-disrupted or -deleted version of B12:03A05 can be created in a strain of S. lividans into which B12:03A05 has been introduced. S. lividans carries a native copy of the gene cluster for CDA. The resulting strain is fermented and analyzed to show that complementation between the CDA PS III and the modified B12:03A05 leads to production of a derivative of daptomycin containing L-trp instead of L-kyn in position 13. In one embodiment of this example, it was shown that a novel lipopeptide could be produced by trans-complementation using the S. lividans TK64/B12:03A05 strain described in Example 7.

To produce the novel lipopeptide, homologous recombination across flanking DNA sequences was used to exchange the bulk of the coding region of dptD in S. lividans TK64/B12:03A05 for a heterologus marker gene. To perform the homologous recombination, two fragments comprising the regions directly upstream (“5′ fragment)” and downstream (“3′ fragment”) of dptD were amplified from chromosomal S. roseosporus DNA using the following primer sets with 5′-terminal extensions in which unique restriction sites have been introduced (underlined):

| | |
|---|---|
| 5′ fragment (1122 bp): | |
| 5′ GCG AAG CTT CTG GTG GCG CAT CAC | (SEQ ID NO: 156) |
| CTG G 3′ | |
| 5′ GCT CTA GAT GGA AGT ATG TCC TCC | (SEQ ID NO: 157) |
| ATC GC 3′ | |
| 3′ fragment (1535 bp): | |
| 5′ CGG ATC CCG CCG GCA CCT GAC | (SEQ ID NO: 158) |
| CC 3′ | |
| 5′ CCG AAT TCC GCC TCC GAG TAC ATC | (SEQ ID NO: 159) |
| GAG G 3′ | |

The amplified fragments were cloned in succession into the corresponding unique sites in the multiple cloning site of pNEB193 (New England Biolabs). The resulting construct, pSD002, was confirmed by restriction digest analysis for orientation, and by sequencing for the absence of errors in the portions generated by PCR. A SpeI fragment containing the marker gene, ermE (erythromycin resistance gene; see Hopwood, supra) was inserted into pSD002 at an XbaI site and verified by restriction digest analysis. The resulting plasmid, pSD005, thus includes a cassette composed of ermE flanked by DNA stretches homologous to DNA sequences upstream and downstream of dptD. Once inserted into the daptomycin biosynthetic gene cluster pathway by homologous recombination, this cassette would essentially replace all of dptD, except for the first 31 bp and the last 12 bp, with ermE. The region comprising the replacement cassette was then subcloned into a vector (a cloning site-modified version of pRHB538; Hosted et al., J. Bacteriol. 179: 180-186, 1997) carrying a temperature-sensitive replication origin and rpsL (a gene conferring sensitivity to streptomycin that can be used in a TK64 background) to create pSD030, the final plasmid in the series for introduction into S. lividans.

The plasmid, pSD030, was introduced into S. lividans by protoplast transformation essentially as described above in Example 7. The transformation mix of protoplasts and cells was gently spread over R2Ye plates and incubated at 30° C. for approximately 16 hours. Each plate was then flooded with 1 mL of water containing 1.25 mg of erythromycin, resulting in a final concentration of 50 μg/ml once the liquid was absorbed into the media. Erythromycin-resistant colonies arising on the transformation plate after 7 days were inoculated into 25 mL of TSB (Hopwood, supra) plus erythromycin and incubated at 30° C. for 48 hours. The mycelium was harvested, and 1/10th of the mycelial mass was macerated and transferred to a new aliquot of 25 mL TSB plus erythromycin. The resultant solution was then incubated at 40° C. to select against the temperature-sensitive replicon of pSD030. After 48 hours, the mycelium was harvested by centrifugation, macerated and resuspended in a final volume of 2 mL TSB. 100 μL of this suspension was spread on SPMR plates (Babcock et al., J. Bacteriol. 170: 2802-2808, 1988) containing 50 μg/mL erythromycin and 30 μg/mL of streptomycin. Colonies that survived were screened and shown to have the correct genotype by PCR to identify strains such as CBUK 137860, in which ermE had successfully replaced dptD.

Starting material of CBUK 137860 was prepared essentially as described in Example 10 and used to produce a seed culture. The production culture was also generated essentially as described in Example 10, but the aeration was at 0.7 vvm. The pH of the fermenter was computer controlled at 6.50 with a 14% (v/v) ammonium hydroxide solution. A 50% (w/v) glucose solution was fed to the culture at 0.36 g/L/hr throughout the fermentation.

The biomass from the 20 L fermentation was discarded and the clarified liquor was applied to an open glass column, packed with Mitsubushi HP20 resin (60×300 mm) and conditioned with methanol and water. Prior to elution, the column was washed with 2 L of water followed by 2 L of methanol/water (1:4). The column was then eluted with 2 L of methanol/water (4:1) followed by 1 L methanol, and collected as two separate fractions.

Liquid chromatography-mass spectroscopy (LC-MS) electrospray ionization (ESI) analysis indicated that both fractions contained the A21978C/CDA hybrid molecules, and the less complex methanol/water (4:1) fraction was processed further. This was evaporated under vacuum to an aqueous residue and then made up to 500 mL with water. It was then back extracted with 3×500 mL of ethyl acetate in a 2 L separating funnel, to give an aqueous and organic fraction. LC-MS (ESI) indicated that the hybrid molecules were absent from the organic phase and it was discarded. The aqueous fraction was lyophilized overnight.

The hybrid molecules were purified by preparative high performance liquid chromatography (HPLC) using a Waters Prep LC system and a Waters 40×200 mm Nova-Pak C18 60 Å 6 μm radially-compressed double cartridge with 40×10 mm guard. The freeze-dried material was dissolved in water and purified using a gradient method. This method held at 90% water and 10% acetonitrile for 2 minutes and was followed by a linear gradient over 13 minutes to 25% water and 75% acetonitrile. The flow was 55 mL/min and the whole gradient was buffered with 0.04% trifluoroacetic acid. Fractions were collected and analyzed by LC-MS on a Finnigan SSQ710c LC-MS system using electrospray ionisation (ESI) in positive ion mode, with a scan range of 200-2000 daltons and 2 second scans. Chromatographic separation for this LC-MS analysis was achieved on a Waters Symmetry C8 column (4.6×50 mm, 3.5 μm particle size) eluted with a linear water-acetonitrile gradient containing 0.01% formic acid, increasing from 10% to 100% acetonitrile over a period of six minutes after a initial delay of 0.5 minutes, then remaining at 100% acetonitrile for a further 3.5 minutes before re-equilibration. The flow rate was 1.5 mL/minute and the method was run at ambient temperature.

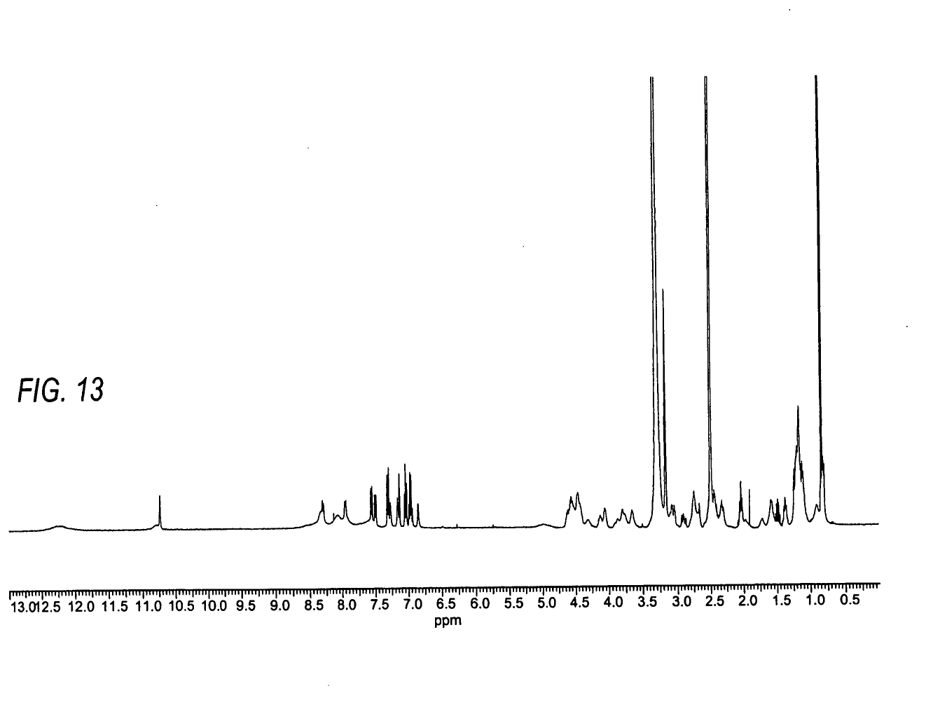

The analysis identified the A21978C/CDA hybrids as the expected analogues of the native A21978C lipopeptides A21978C1, with a branched native C₁₁ acyl chain, and A21978C2, with a branched C₁₂ acyl chain, in which the native kynurenine residue is replaced with a tryptophan residue. Both fractions required further purification prior to NMR studies. The C₁₁ hybrid (A) was further purified using an isocratic method with 60% water and 40% acetonitrile buffered with 0.04% trifluoroacetic acid. 1.8 mg of material was isolated. The C₁₂ hybrid (B) final purification step used an isocratic method with 58% water and 42% acetonitrile buffered with 0.04% trifluoroacetic acid. Approximately 1.5 mg of material was isolated. A 1H NMR spectrum is shown in FIG. 13. The UV maxima and ESI-MS molecular ion information (doubly-charged ions observed in negative ion mode) for A and B are presented below:

| | A | B |
|---|---|---|
| ESI-MS (m/z) | 814 (M − 2H)²⁻ | 821 (M − 2H)²⁻ |
| UV-vis λₘₐₓ/nm | 221, 280, | 221, 280 |

Similar experiments where modified versions of the daptomycin NRPS (e.g. dptA deletion, dptA plus dptD deletion, etc.) are introduced into other secondary metabolite producing strains such as S. lividans, S. fradiae, S. viridochromogenes and others may also generate derivative compounds based on the daptomycin backbone. Given that NRPS subunits can be expressed separately and exchanged, one may trans-complement or exchange all subunits, alone or in combination, with one or more natural or modified NRPS subunits (including modified versions of daptomycin synthetase as described above) in S. roseosporus or other expressing hosts. As an example, the following modified S. roseosporus strain can be created: dptA and dptD deleted from the native locus, dptBC expression intact. Then, this strain can be complemented by an ectopically integrated dptA that is modified by site-directed mutagenesis to incorporate asp instead of asn at position 2 (Example 12A) and an ectopically integrated dptD modified so that its kyn-accepting module is exchanged for the trp-accepting module of CDA PSIII or by an ectopically integrated CDAIII. Another way to create the same strain is to bring a dptA, dptD deleted BAC B12:03A05 into a S. lividans TK64 derivative that already carries a modified dptA subunit that incorporates aspartate instead of asparagine. Such strains may be fermented to recover a daptomycin derivative with asp in position 2, and trp in position 13.

D. Insertion of One or More Modules to cause the Expansion of the Daptomycin Ring or Lengthening of the Tail.

A simple NRPS elongation module may be defined as comprising domains “C-A-T” (condensation-, adenylation- and thiolation-domains). To link modules, and to identify a permissive site within the daptomycin NRPS in which to insert additional internal modules, the domain and inter-domain regions are examined for sequences indicative of flexible “linker” sequences. See, e.g., Mootz et al., Proc. Natl. Acad. Sci. U.S.A., 97, pp. 5848-5853 (2000), which is incorporated herein by reference in its entirety. Sequences encoding an additional module are inserted in the linker sequence between an upstream T-domain and a downstream C-domain using well-known genetic recombination techniques, e.g., see Example 11A, above.

Isolation of the module DNA is obtained from the chromosomal DNA extracted from the producer organism. Various isolation techniques can be used such as, cutting the chromosomal DNA with restriction enzymes and isolating a fragment coding for the module(s) of interest after it is identified by means of Southern blot or isolation of the module(s) of interest by e.g. genetic amplification (PCR) using suitable primers. Sequencing and characterization of the amplified fragments as well as cloning can be performed according to conventional techniques. New modules can be inserted between the modules specifying L-Thr and Gly in dptA; between the modules specifying L-Orn and L-Asp or L-Asp and D-Ala in dptBC; between L-Asp and Gly or Gly and D-Ser in dptBC; and between modules specifying 3-MG and L-Kyn in dptD to expand the ring of daptomycin. New modules can be inserted in the dptA gene between the modules specifying L-Trp and D-Asn, D-Asn and L-Asp, or L-Asp and L-Tyr to lengthen the tail of daptomycin. The module(s) insertions can be carried out using the methods for double crossovers described in Example 11A.

E. Insertion of an Additional Carboxyl Terminus Module Adjacent to and Upstream from the Thioesterase Module.

Carboxy-terminal thioesterase domains (“Te-domains”) of a variety of NRPSs and PKSs can cleave (i.e., catalyze chain termination) non-natural peptide and polyketide substrates. See Mootz et al., supra; see also de Ferra et al., J. Biol. Chem., 272, 25304-25309 (1997); each of which is hereby incorporated by reference in its entirety. Te-domains can act as hydrolases, releasing a linear product, or as cyclases, releasing cyclic peptides. Evidence suggests that a Te-domain which functions as a cyclase in its natural configuration within a NRPS or PKS may, nonetheless, function as a hydrolase when engineered into new modular configurations. (An isolated C-terminal Te-domain has been shown to catalyze cyclization on various substrates as long as key “recognition amino acids” are at the C- and N-termini of the substrate; see Trauger et al., Nature, 407, pp. 215-218, 2000.

It has also been shown that some C-terminal Te-domains function best, when moved, by retaining their association with a portion of the protein domain occurring directly upstream in the natural NRPS or PKS modular configuration. See Guenzi et al., J. Biol. Chem., 273, pp. 14403-14410 (1998), incorporated herein by reference in its entirety. It is possible that retaining the boundary between the Te-domain and a portion of the domain directly upstream (N-terminal) may also contribute to retaining cyclase function of the Te-domain within a new modular configuration.

Accordingly, to insert an additional module upstream from a Te-domain and have it be operatively linked thereto, one can identify linker sequences between the C-A-T modules and the C-terminal Te-domain, as described above, and insert sequences encoding the additional module therein, using standard genetic manipulations. Optionally, one can engineer a new, hybrid C-terminal Te-domain in which the C-terminal portion of the penultimate thiolation (T-) domain remains linked (or is otherwise grafted) to the Te-domain (“T-/Te-domain”). See Guenzi et al., 1998, supra. Sequences encoding the additional module are then inserted within the identified linker region upstream from a hybrid T-/Te domain using well-known genetic recombination techniques, as described in Example 11A, above.

F. Internal Deletion of One or More Modules to Cause the Contraction of the Daptomycin Ring or Shortening of the Tail.

To obtain a deletion of an internal module(s) on the chromosome by double crossing-over and selection on antibiotic plates it is necessary to prepare a plasmid containing a fragment of chromosomal DNA situated upstream from the module to be deleted fused by ligation to a fragment downstream of the module(s) to be deleted. The plasmid also carries a wild type rpsL gene to confer streptomycin sensitivity on recombinants in a streptomycin-resistant genetic background (see Example 11A), an antibiotic resistance gene (e.g., apramycin resistance, thiostrepton resistance or hygromyicin resistance) for selection of single crossovers, and a temperature sensitive replicon that can be cured at elevated temperature. A single crossover inserting the plasmid by homologous recombination into the region of DNA upstream of the module(s) to be exchanged can be selected for antibiotic resistance at elevated temperature. The second crossover that deletes the module(s) can then be selected on media containing streptomycin (thus eliminating all plasmid sequences). Recombinants containing deletions of the appropriate module(s) can be verified by Southern blot hybridization of S. roseosporus DNA cleaved with appropriate restriction endonucleases. This approach can be taken to delete the L-Asp module or the Gly module from dptBC, for example. It can also be used to delete the modules in the dptA gene specifying L-Asn, L-Asp or both L-Asn and L-Asp together.

G. Translocation of the Terminal Thioesterase Module to Cause the Contraction of the Daptomycin Ring.

Sequences encoding the thioesterase (Te) region which resides at the carboxyl terminus of the last module in the daptomycin NRPS (DptD) may be translocated upstream to the end of an internal module encoding region. This translocation will result in the release of a defined shortened product that will yield a truncated linear or cyclic peptide. The translocation of the Te can be accomplished by double crossovers much the same way as described above in Examples 12A and 12F.

Molecular Exchange Between Daptomycin NRPS and Other NRPS or PKS Genes

a. Daptomycin Thioesterase onto Different NRPS or PKS

Using well-known molecular and genetic methods such as those described above, sequences encoding a C-terminal Te-domain of the daptomycin NRPS of the invention (e.g., DptD) may be moved (either alone or in combination with one or more upstream modules or portions thereof) into association with sequences encoding other NRPS or PKS modular genes from a variety of other hosts to produce hybrid modular synthetases that are capable of producing new peptide and/or hybrid peptide/polyketide products having useful properties. See, e.g., Stachelhaus et al., Science, 269, pp. 69-72 (1995) and Cane and Khosla, Chem. Biol., 6, pp. 319-325 (1999); each of which is incorporated herein by reference in its entirety. Similarly, daptomycin sequences encoding a free thioesterase of the invention (e.g., DptH) may be moved into association other NRPS or PKS encoding modular genes to produce hybrid modular synthetases.

b. Module and Domain Exchanges Between Daptomycin and Other NRPS and/or PKS

Various sequences derived from the daptomycin biosynthetic gene cluster of the invention—including but not limited to domains and modular structures—may be used to construct plasmids and other vectors for use in genetic recombination reactions (gene duplication, conversion, replacement, etc.) between daptomycin sequences and natural or synthetic NRPS and PKS sequences in homologous and heterologous hosts to produce hybrid NRPS and hybrid NRPS/PKS modular synthetases comprising sequences from the daptomycin biosynthetic gene cluster. Such hybrid synthetases will produce novel peptide and polyketide products which are expected to have new and useful properties.

Creation of Lipopeptide Derivatives of Nonribosomally-Synthesized Peptides that are not Normally Acylated.

The fatty acid tail of daptomycin is thought to be attached by the products of the dptE and dptF genes, working in conjunction with the condensation domain at the start of dptA. These genes and gene fragments may be transferred to the beginning of a foreign nonribosomal peptide synthase gene, or to an internal location within the daptomycin gene cluster, either at the start of a gene (e.g. 5′ of dptBC or dptD) or within a gene at the start of a module (e.g. 5′ of module 2), to create acylated versions of the foreign nonribosomally synthesized peptide, or to create acylated, truncated derivatives of daptomycin. The foreign gene may be derived from another natural organism, or one generated by recombinant techniques, e.g. various versions of daptomycin that have undergone modifications to expand or contract the ring, to have substituted amino acids in the peptide sequence as described herein.

Modification of Amino Acid Stereoisomers in the Peptide Structure.

Stereospecificity in the amino acid backbone produced by an NRPS is determined by the presence of epimerase domains in the donor module and distinctive condensation domains in the acceptor module. An alteration in stereochemistry of the amino acids may be achieved by addition of an epimerase domain to a donor module, and substitution of the appropriate condensation domain to the acceptor module. An alteration can also be made by removal of the epimerase domain from a donor module, and the substitution of the appropriate condensation domain in the acceptor, e.g. the epimerase domain can be excised from module 2 of dptA, and the condensation domain of module 3 of dptA can be exchanged for the condensation domain from another module that does not normally accept a D-amino acid. Useful epimerase and condensation domains may be found in the daptomycin cluster as well as in other nonribosomal peptide synthetase genes.

### EXAMPLE 13

### Use of Free Thioesterase

A. Expression of dptD or dptH Related Sequences in Homologous or Heterologous Systems to Increase Efficiency of Product Formation by Modular NRPSs and PKSs

The C-terminal Te-domain excised from tyrocidine synthetase has been shown to catalyze cyclization on various peptide substrates as long as key “recognition amino acids” are at the C- and N-termini of the substrate. See Trauger et al., Nature, 407, 215-218, 2000. Sequences derived from the C-terminal domain of daptomycin NRPS (e.g., dptD) may similarly be isolated and expressed—alone or in the form of suitable fusion proteins—in a homologous or heterologous host (or in vitro system) to catalyze cyclization of peptide and polyketide products which naturally (or which have been engineered to) possess key substrate recognition amino acids required for the daptomycin Te-domain to bind and join substrate ends (see below).

When isolating sequences derived from the C-terminal Te-domain of daptomycin synthetase (NRPS) for independent expression, it may be preferable to include natural C-terminal sequences from the penultimate amino acid module. See, e.g., Guenzi et al., 1998, supra. Various dptD and upstream-derived sequence combinations can be tested using techniques well-known in the art to optimize the thioesterase activity of the C-terminal Te-domain of daptomycin NRPS when expressed independently from upstream polypeptides such as DptA and/or DptBC. Independent expression of the C-terminal Te-domain of daptomycin may be accomplished using standard molecular biology techniques. Independent expression of the C-terminal Te-domain of daptomycin NRPS is accomplished by inserting sequences derived from the thioesterase domain of the dptD ORF (SEQ ID NO:3) downstream from natural daptomycin NRPS promoter sequences (SEQ ID NO:2) in an appropriately constructed expression vector. Alternatively, independent expression of the C-terminal Te-domain of daptomycin NRPS is accomplished by inserting the thioesterase domain of the dptD ORF (SEQ ID NO:3) downstream from a heterologous promoter, which is constitutively active or from a heterologous promoter which may be turned on or off in a regulated manner. Those of skill in the art will appreciate the factors to be considered in selecting appropriate promoters and vectors for expression or over-expression in a host-dependent manner.

Sequences derived from the free thioesterase domain of the daptomycin biosynthetic gene cluster of the invention (dptH) may be similarly expressed in a homologous or heterologous host to test and develop novel cyclic peptides and the like.

The key recognition amino acids of daptomycin are identified by systematic mutagenesis of the amino acid residues of daptomycin followed by cyclization assays using each modified daptomycin substrate in a reaction catalyzed by the isolated Te-domain. C- and N-terminal amino acid residues required for daptomycin cyclization are identified and engineered into new substrate backbones into which peptide and polyketide building block units can be inserted. Substrate engineering can be performed at the nucleic acid sequence level or at the peptide level using techniques well-known to those of skill in the art. The length and composition of preferred substrates may be determined empirically, taking into consideration factors well-known to the skilled worker and including (but not limited to) substrate binding efficiency, catalytic rate, biological activity of resulting cyclic product(s), and ease of purification of the final products.

B. Mutagenize dptD or dptH to Affect Proofreading Function

The dptH gene from the daptomycin gene cluster is related to free thioesterase enzymes which are known to participate in the biosynthesis of some peptide and polyketide secondary metabolites. See e.g., Schneider and Marahiel, Arch. Microbiol., 169, pp. 404-410 (1998), and Butler et al., Chem. Biol., 6, pp. 87-292 (1999), hereby incorporated by reference in their entirety. It has been suggested that editing thioesterases are often required for efficient natural product synthesis. Butler et al. have postulated that the free thioesterase found in the polyketide tylosin gene cluster may be involved in editing and proofreading functions, consistent with the suggested role of the thioesterases in efficient product formation.

Homologous or heterologous expression of the daptomycin dptH (encoding a free thioesterase) or the thioesterase-encoding domain of dptD (encoding the C-terminal Te) genes may affect the efficiency of product formation by modular NRPSs and PKSs. The proposed editing and proofreading functions of the daptomycin thioesterase type II enzyme (DptH) (and potentially of the type I thioesterase enzyme when detached from the C-terminus of the daptomycin gene cluster and separately expressed) may be altered by conventional mutagenesis and other recombinant DNA techniques, e.g., those known to affect adversely the fidelity of DNA replication. Altered and mutated forms of thioesterase genes may be expressed in appropriate expression systems and screened for those which encode thioesterase enzymes having altered biological properties. Especially desirable would be thioesterase enzymes that have higher than normal rates of amino acid misincorporation. Such mutants would be useful for creating a larger diversity of peptide and peptide/polyketide hybrid products having new and useful biological properties.

### EXAMPLE 14

### Using Daptomycin Biosynthetic Genes to Identify and Isolate Related Genes

The nucleic acid and amino acid sequences of the invention can be compared to the corresponding sequences from another lipopeptide pathway in order to identify features that can then be used to identify sequences from an NRPS or a component of an NRPS encoding another lipopeptide.

The amino acid 3-methyl glutamic acid (3MG) is uncommon, but is found in daptomycin, the calcium dependent antibiotic (CDA) from S. coelicolor, and the A54145 compound made by S. fradiae. Comparison of the S. roseosporus and S. coelicolor nucleic acid sequences that encode the 3MG adenylation domain, as well as from analogous sequences from genes that adenylate other amino acids, were used to create the primer pair P140 and P141:

| | |
|---|---|
| P140 | |
| ACSSWSGGSGTSSCCTTCATGAA | (SEQ ID NO: 160) |
| P141 | |
| ATGGTGTTCGAGAACTAYCC. | (SEQ ID NO: 161) |

S. fradiae cosmid library clones were screened by PCR using P140 and P141 using standard techniques. The PCR reaction yielded a nucleic acid molecule product of approximately 700 bp, whose sequence proved similar to the region encoding the 3MG adenylation domain in S. roseosporus and S. coelicolor. Extension of the sequence by primer walking confirmed that the region identified was the 3MG module in A54145.

This method was also used to identify portions of an NRPS pathway that encode condensation domains downstream of a D-amino acid activating module. D-amino acids are unusual amino acids found in non-ribosomally synthesized peptides, and primers for condensation domains associated with them can be used to identify pathways with such amino acids. The nucleic acid sequences of the S. roseosporus daptomycin and S. coelicolor CDA sequences that encode these D-amino acid condensation domains were compared to each other and to analogous sequences from other condensation domains associated with L-amino acids in order to create the primer pair P144 and P145:

| | |
|---|---|
| P144 | |
| SCSCTSCAGGAGGGSHTSSTSTTCC | (SEQ ID NO: 162) |
| P145 | |
| CCGAASACSACGTCGTCSCGSCC. | (SEQ ID NO: 163) |

S. fradiae cosmid library clones were screened by PCR using P144 and P145 using standard techniques. The PCR reaction yielded a nucleic acid molecule products of approximately 800 basepairs, the sequences of which proved to be similar to the condensation domains following the D-amino acids in S. roseosporus and S. coelicolor. Sequences corresponding to more than one domain were obtained, indicating that the pathway had more than one D-amino acid.

These approaches, based on understanding the sequence of the daptomycin pathway, can be used to develop special primer sets for other genetic features of lipopeptide pathway gene clusters, such as regions encoding epimerase domains or the condensation domain of the first adenylation module responsible for condensing the fatty acid to the peptide, as well as genes involved in acylation, such as DptE and F.

TABLE 5

| | Nucleotide | Amino Acid |
|---|---|---|
| ORF# - | Sequence | Sequence |
| Fragment | SEQ ID NO: | SEQ ID NO: |
| 1 - 90 kb* | 20 | 19 |
| 2 - 90 kb | 22 | 21 |
| 3 - 90 kb | 24 | 23 |
| 4 - 90 kb | 26 | 25 |
| 5 - 90 kb | 28 | 27 |
| 6 - 90 kb | 30 | 29 |
| 7 - 90 kb | 32 | 31 |
| 8 - 90 kb | 34 | 33 |
| 9 - 90 kb | 36 | 35 |
| 10 - 90 kb | 38 | 37 |
| 11 - 90 kb | 40 | 39 |
| 12a - 90 kb | 42 | 41 |
| 12b - 90 kb | 44 | 43 |
| 13 - 90 kb | 46 | 45 |
| 14 - 90 kb | 48 | 47 |
| 15 - 90 kb | 50 | 49 |
| 16 - 90 kb | 52 | 51 |
| 17 - 90 kb | 54 | 53 |
| 18 - 90 kb | 56 | 55 |
| 19 - 90 kb | 58 | 57 |
| 20 - 90 kb | 60 | 59 |
| 21 - 90 kb | 62 | 61 |
| 22 - 90 kb | 64 | 63 |
| 23 - 90 kb | 66 | 65 |
| 24 - 90 kb | 68 | 67 |
| 25 - 90 kb | 70 | 69 |
| 26a - 90 kb | 72 | 71 |
| 26b - 90 kb | 74 | 73 |
| 27 - 90 kb | 76 | 75 |
| 28 - 90 kb | 78 | 77 |
| 29 - 90 kb | 16 | 15 |
| dptE | | |
| 30 - 90 kb | 18 | 17 |
| dptF | | |
| 31 - 90 kb | 10 | 9 |
| dptA | | |
| 32 - 90 kb | 12 | 11 |
| dptB | | |
| 33 - 90 kb | 14 | 13 |
| dptC | | |
| 34 - 90 kb | 3 | 7 |
| dptD | | |
| 35 - 90 kb | 80 | 79 |
| 36 - 90 kb | 6 | 8 |
| dptH | | |
| 37 - 90 kb | 82 | 81 |
| 38 - 90 kb | 84 | 83 |
| 41 - 90 kb | 105 | 104 |
| 1 - SP6 | 86 | 85 |
| 2 - SP6 | 88 | 87 |
| 3 - SP6 | 90 | 89 |
| 4 - SP6 | 92 | 91 |
| 5 - SP6 | 94 | 93 |
| 6 - SP6 | 96 | 95 |
| 7 - SP6 | 98 | 97 |
| 8 - SP6 | 100 | 99 |
| 9 - SP6 | 102 | 101 |
| 2 - GTC2 | 107 | 108 |
| 3 - GTC2 | 109 | 110 |
| 4 - GTC2 | 111 | 112 |
| 5 - GTC2 | 113 | 114 |
| 6 - GTC2 | 115 | 116 |
| 7 - GTC2 | 117 | 118 |
| 8 - GTC2 | 119 | 120 |
| 9 - GTC2 | 121 | 122 |
| 10 - GTC2 | 123 | 124 |
| 11 - GTC2 | 125 | 126 |
| 12 - GTC2 | 127 | 128 |
| 13 - GTC2 | 129 | 130 |
| 14 - GTC2 | 131 | 132 |
| 15 - GTC2 | 133 | 134 |
| 16 - GTC2 | 135 | 136 |
| *ORF-1 of the 90 kb fragment is a partial sequence of the ORF because the 3′ end of the ORF, including the stop codon, terminates in the SP6 fragment. The nucleic acid sequence of the 3′ end of the ORF-1 sequence, including the stop codon, corresponds to nucleotides 13020-12876 of | | |
| # SEQ ID NO: 103. Thus, the full open reading frame of ORF-1 of the 90 kb fragment consists of SEQ ID NO: 19 (the complementary strand of nucleotides 1635-1 of SEQ ID NO: 1) followed by the complementary strand of nucleotides 13020-12876 of SEQ ID NO: 103. | | |

TABLE 6
BlastX Results for ORFs in 90 kb Fragment

| ORF | Start | Stop | Str | BLASTX (accession numbers, entry title, P-value, E-value) | Polypeptide |
|---|---|---|---|---|---|
| 1 | 1637 | 1 | − | emb\|CAB88932.1\| (AL353863) putative ABC transporter [Strept . . . 732 0.0 | Type III ABC transporter |
| | | | | pir\|\|S57562 strW protein - Streptomyces glaucescens >gi\|212 . . . 330 e−114 | similar to Streptomyces |
| | | | | emb\|CAB88932.1\| (AL353863) putative ABC transporter [Streptomyces coelicolor A3(2)] | glaucescens strW gene |
| | | | | Length = 593 | (resistance to |
| | | | | Score = 732 bits (1870), Expect(2) = 0.0 | streptomycin); has Walker |
| | | | | Identities = 367/462 (79%), Positives = 405/462 (87%) | A, B motifs. Translationally |
| | | | | | coupled to Orf2. |
| 2 | 3502 | 1634 | − | emb\|CAB88931.1\| (AL353863) putative ABC transporter transme. 854 0.0 | ABC transporter similar to |
| | | | | pir\|\|S57561 strV protein - Streptomyces glaucescens >gi\|212 320 4e−86 | Streptomyces glaucescens |
| | | | | emb\|CAB88931.1\| (AL353863) putative ABC transporter transmembrane | strV gene (resistance to |
| | | | | subunit [Streptomyces coelicolor A3(2)] | |
| | | | | Length = 623 | streptomycin); has Walker |
| | | | | Score = 854 bits (2183), Expect = 0.0 | B motif. Translationally |
| | | | | Identities = 456/637 (71%), Positives = 510/637 (79%), Gaps = 17/637 (2%) | coupled to Orf1. |
| 3 | 5144 | 3659 | − | gi\|3913215 1-CARBOXY-3-CHLORO-3,4-DIHYDROXYCYCLO HE 158 1.6e−10 | Oxidoreductase |
| | | | | gi\|3914351 PUTATIVE 4,5,-DIHYDROXYPHTHALATE DEHYDRO 120 4.6e−06 | |
| | | | | gi\|3913215\|sp\|Q44258\|CBAC_ALCSB | |
| | | | | 1-CARBOXY-3-CHLORO-3,4-DIHYDROXYCYCLO HEXA-1,5-DIENE | |
| | | | | DEHYDROGENASE | |
| | | | | Length = 397 | |
| | | | | Score = 158 (66.0 bits), Expect = 1.6e−10, P = 1.6e−10 | |
| | | | | Identities = 59/218 (27%), Positives = 180/218 (82%), Gaps = 24/218(11%) | |
| 4 | 8364 | 5410 | − | gi\| 2506961 D-LACTATE DEHYDROGENASE [CYTOCHROME], MI . . . 251 5.1e−21 | Transmembrane, FAD- |
| | | | | gi\|3023651 D-LACTATE DEHYDROGENASE [CYTOCHROME] PRE . . . 212 1.9e−16 | dependent |
| | | | | gi\|2506961\|sp\|P32891\|DLD1_YEAST | dehydrogenase |
| | | | | D-LACTATE DEHYDROGENASE [CYTOCHROME], MITOCHONDRIAL | |
| | | | | PRECURSOR (D-LACTATE FERRICYTOCHROME C OXIDOREDUCTASE) | |
| | | | | (D-LCR) | |
| | | | | Length = 587 | |
| | | | | Score = 251 (102.2 bits), Expect = 5.1e−21, P = 5.1e−21 | |
| | | | | Identities = 119/502 (23%), Positives = 374/502 (74%), Gaps = 91/502 (18%) | |
| 5 | 8916 | 8416 | − | gi\|10803169\|emb\|CAC13097.1\| (AL445503) putative marR-family . . . 107 3e−23 | Mar family-related protein |
| | | | | gi\|15896528\|ref\|NP_349877.1\| Transcriptional regulator, Mar . . . 56 1e−07 | Transcriptional regulator |
| | | | | gi\|10803169\|emb\|CAC13097.1\| (AL445503) putative marR-family regulator | Involved in antibiotic |
| | | | | [Streptomyces coelicolor] | susceptibility and resistance |
| | | | | Length = 153 | |
| | | | | Score = 107 bits (268), Expect = 3e−23 | |
| | | | | Identities = 66/110 (60%), Positives = 79/110 (71%) | |
| 6 | 9030 | 10853 | + | Gb\|AAF67494.1\|AF170880_1 (AF170880) NovA [Streptomyces sphe 1017 0.0 | NovA-related protein |
| | | | | emb\|CAC13096.1\| (AL445503) putative ABC transporter ATP-bin 946 0.0 | (novobiocin biosynthetic |
| | | | | gb\|AAF67494.1\|AF170880_1 (AF170880) NovA [Streptomyces spheroides] | gene cluster) that is ABC |
| | | | | Length = 635 | transporter; has Walker A, |
| | | | | Score = 1017 bits (2602), Expect = 0.0 | B motifs |
| | | | | Identities = 526/609 (86%), Positives = 559/609 (91%), Gaps = 3/609(0%) | |
| 7 | 10933 | 11544 | + | emb\|CAB91142.1\| (AL355913) putative translation initiation . . . 64 3e−09 | Hypothetical protein with |
| | | | | pir\|\|JQ0405 hypothetical 119.5K protein (uvrA region) - Mic . . . 62 7e−09 | no significant match |
| | | | | emb\|CAB91142.1\| (AL355913) putative translation initiation factor | identified by BlastX |
| | | | | IF-2(fragment)[Streptomyces coelicolor A3(2)] | |
| | | | | Length = 835 | |
| | | | | Score = 63.6 bits (152), Expect = 3e−09 | |
| | | | | Identities = 74/237 (31%), Positives = 84/237 (35%), Gaps = 6/237 (2%) | |
| 8 | 11990 | 12850 | + | gi\|7688708\|gb\|AAF67495.1\|AF170880_2 (AF170880) NovB [Strept 319 2e−86 | NovB-related protein |
| | | | | gi\|10803167\|emb\|CAC13095.1\| (AL445503) conserved hypothetic 297 9e−80 | (novobiocin biosynthetic |
| | | | | gi\|7688708\|gb\|AAF67495.1\|AF170880_2 (AF170880) NovB [Streptomyces spheroides] | gene cluster) |
| | | | | Length = 284 | |
| | | | | Score = 319 bits (817), Expect = 2e−86 | |
| | | | | Identities = 156/247 (63%), Positives = 188/247 (75%) | |
| 9 | 14038 | 12878 | − | gb\|AAF67496.1\|AF170880_3 (AF170880) NovC [Streptomyces sphe 520 e−146 | Nov-C related protein that |
| | | | | emb\|CAB71851.1\| (AL138667) putative monooxygenase. [Strepto 261 1e−68 | is oxidoreductase |
| | | | | gb\|AAF67496.1\|AF170880_3 (AF170880) NovC [Streptomyces spheroides] | |
| | | | | Length = 352 | |
| | | | | Score = 520 bits (1324), Expect = e−146 | |
| | | | | Identities = 260/346 (75%), Positives = 283/346 (81%), Gaps = 1/346 (0%) | |
| 10 | 14348 | 14070 | − | pir\|\|I39929 hypothetical protein orfM - Bacillus subtilis 78 2e−14 | Monooxygenase |
| | | | | pir\|\|D69817 sulfate starvation-induced protein 6 homolog yg 78 2e−14 | |
| | | | | pir\|\|I39929 hypothetical protein orfM - Bacillus subtilis (fragment) | |
| | | | | gb\|AAA64350.1\| (L16808) Gene disrupted by Tn917 insertion after base 3033. | |
| | | | | Translation product hydrophilic, no | |
| | | | | homologues in the databases.; putative [Bacillus subtilis] | |
| | | | | Length = 372 | |
| | | | | Score = 78.0 bits (189), Expect = 2e−14 | |
| | | | | Identities = 37/53 (69%), Positives = 41/53 (76%) | |
| 11 | 15697 | 14522 | − | gi\|1723069 HYPOTHETICAL 69.5 KDA PROTEIN RV1364C 86 0.04 | Hypothetical protein |
| | | | | gi\|8928323 SIGMAB REGULATION PROTEIN PHOSPHATASE 2C 85 0.053 | |
| | | | | gi\|1723069\|sp\|Q11034\|YD64_MYCTU HYPOTHETICAL 69.5 KDA PROTEIN RV1364C | |
| | | | | Length = 653 | |
| | | | | Score = 86 (37.9 bits), Expect = 0.041, P = 0.04 | |
| | | | | Identities = 45/153 (29%), Positives = 132/153 (86%), Gaps = 6/153 (3%) | |
| 12a | 17597 | 16938 | − | gi\|728850 GLUCOAMYLASE S1/S2 PRECURSOR (GLUCAN 1,4 113 1.9e−05 | Hypothetical protein |
| | | | | gi\|138350 GLYCOPROTEIN X PRECURSOR 91 0.0072 | |
| | | | | gi\|728850\|sp\|P08640\|AMYH_YEAST | |
| | | | | GLUCOAMYLASE S1/S2 PRECURSOR (GLUCAN | |
| | | | | 1,4-ALPHA-GLUCOSIDASE) (1,4-ALPHA-D-GLUCAN GLUCOHYDROLASE) | |
| | | | | Length = 1367 | |
| | | | | Score = 113 (48.4 bits), Expect = 1.9e−05, P = 1.9e−05 | |
| | | | | Identities = 47/186 (25%), Positives = 158/186 (84%), Gaps = 12/186 (6%) | |
| 12b | 17870 | 18682 | + | gi\|8546911\|emb\|CAB94663.1\| (AL359216) hypothetical protein . . . 34 1.3 | Hypothetical Protein |
| | | | | gi\|8546913\|emb\|CAB94625.1\| (AL359215) putative membrane pro . . . 33 2.9 | |
| | | | | gi\|8546911\|emb\|CAB94663.1\| (AL359216) hypothetical protein SC1D2.05 (fragment). | |
| | | | | [Streptomyces coelicolor A3(2)] | |
| | | | | Length = 192 | |
| | | | | Score = 34.3 bits (77), Expect = 1.3 | |
| | | | | Identities = 28/94 (29%), Positives = 40/94 (41%), Gaps = 5/94 (5%) | |
| 13 | 19898 | 18915 | − | emb\|CAB94641.1\| (AL359215) putative iron transport lipoprot . . . 250 2e−65 | Iron (ABC) transporter |
| | | | | pir\|\|C83282 hypothetical protein PA2913 [imported] - Pseudo . . . 168 1e−40 | Association with orfs 14 |
| | | | | emb\|CAB94641.1\| (AL359215) putative iron transport lipoprotein. | and 15 |
| | | | | [Streptomyces coelicolor A3(2)] | |
| | | | | Length = 345 | |
| | | | | Score = 250 bits (632), Expect = 2e−65 | |
| | | | | Identities = 133/322 (41%), Positives = 188/322 (58%), Gaps = 13/322 (4%) | |
| 14 | 20674 | 19907 | − | emb\|CAB94640.1\| (AL359215) putative iron transport protein, . . . 279 3e−74 | Iron transporter |
| | | | | emb\|CAC14366.1\| (AL445963) Fe uptake system permease [Strep . . . 250 2e−65 | Association with orfs 13 |
| | | | | emb\|CAB94640.1\| (AL359215) putative iron transport protein, ATP-binding component. | and 15 |
| | | | | [Streptomyces coelicolor A3(2)] | |
| | | | | Length = 258 | |
| | | | | Score = 279 bits (706), Expect = 3e−74 | |
| | | | | Identities = 141/251 (56%), Positives = 181/251 (71%) | |
| 15 | 21782 | 20676 | − | emb\|CAB94639.1\| (AL359215) putative FecCD-family membrane t 371 e−102 | Iron transporter |
| | | | | emb\|CAC14365.1\| (AL445963) Fe uptake system integral membra 277 2e−73 | Association with orfs 13 |
| | | | | emb\|CAB94639.1\| (AL359215) putative FecCD-family membrane transport protein. | and 14 |
| | | | | [Streptomyces coelicolor A3(2)] | |
| | | | | Length = 368 | |
| | | | | Score = 371 bits (943), Expect = e−102 | |
| | | | | Identities = 192/365 (52%), Positives = 248/365 (67%) | |
| 16 | 23130 | 21877 | − | gi\|138350 GLYCOPROTEIN X PRECURSOR 94 0.0088 | Hypothetical protein |
| | | | | gi\|728850 GLUCOAMYLASE S1/S2 PRECURSOR (GLUCAN 1,4 . . . 83 0.16 | |
| | | | | gi\|138350\|sp\|P28968\|VGLX_HSVEB GLYCOPROTEIN X PRECURSOR | |
| | | | | Length = 797 | |
| | | | | Score = 94 (41.0 bits), Expect = 0.0088, P = 0.0088 | |
| | | | | Identities = 51/216 (23%), Positives = 181/216 (83%), Gaps = 9/216 (4%) | |
| 17 | 23987 | 23127 | − | gi\|14591289\|ref\|NP_143367.1\| hypothetical protein [Pyrococc . . . 46 3e−04 | Hypothetical protein |
| | | | | gi\|322598\|pir\|\|S28604 St12p protein - Arabidopsis thaliana 42 0.006 | |
| | | | | gi\|14591289\|ref\|NP_143367.1\| hypothetical protein [Pyrococcus horikoshii] | |
| | | | | Length = 248 | |
| | | | | Score = 46.2 bits (108), Expect = 3e−04 | |
| | | | | Identities = 31/119 (26%), Positives = 62/119 (52%), Gaps = 2/119 (1%) | |
| 18 | 24966 | 23953 | − | gi\|543960 CYSTATHIONINE BETA-SYNTHASE (SERINE SULF 162 4.3e−11 | Hypothetical protein |
| | | | | gi\|2493892 CYSTEINE SYNTHASE (O-ACETYLSERINE SULFHY . . . 147 2.4e−09 | |
| | | | | gi\|543960\|sp\|P32232\|CBS_RAT CYSTATHIONINE BETA-SYNTHASE (SERINE | |
| | | | | SULFHYDRASE) (BETA-THIONASE) (HEMOPROTEIN H-450) | |
| | | | | Length = 561 | |
| | | | | Score = 162 (67.5 bits), Expect = 4.3e−11, P = 4.3e−11 | |
| | | | | Identities = 76/290 (26%), Positives = 243/290 (83%), Gaps = 17/290 (5%) | |
| 19 | 25228 | 26127 | + | gi\|8928195 MEVALONATE KINASE (MK) 99 0.00096 | Hypothetical protein |
| | | | | gi\|8928178 MEVALONATE KINASE (MK) 90 0.011 | |
| | | | | gi\|8928195\|sp\|Q9V187\|KIME_PYRAB MEVALONATE KINASE (MK) | |
| | | | | Length = 335 | |
| | | | | Score = 99 (43.0 bits), Expect = 0.00096, P = 0.00096 | |
| | | | | Identities = 25/61 (40%), Positives = 49/61 (80%) | |
| 20 | 26445 | 27212 | + | gi\|731172 SKIN SECRETORY PROTEIN XP2 PRECURSOR (AP . . . 87 0.019 | Hypothetical protein |
| | | | | gi\|127749 MYOSIN IC HEAVY CHAIN 86 0.025 | |
| | | | | gi\|731172\|sp\|P17437\|XP2_XENLA SKIN | |
| | | | | SECRETORY PROTEIN XP2 PRECURSOR (APEG PROTEIN) | |
| | | | | Length = 439 | |
| | | | | Score = 87 (38.3 bits), Expect = 0.019, P = 0.019 | |
| | | | | Identities = 20/54 (37%), Positives = 39/54 (72%) | |
| 21 | 28124 | 27381 | − | emb\|CAB56736.1\| (AL121600) ABC transport protein, ATP-bindi . . . 351 4e−96 | ABC Transporter (Mn |
| | | | | pir\|\|H75293 probable manganese ABC transporter, ATP-binding . . . 154 1e−36 | transporter) |
| | | | | emb\|CAB56736.1\| (AL121600) ABC transport protein, ATP-binding subunit | |
| | | | | [Streptomyces coelicolor A3(2)] | |
| | | | | Length = 252 | |
| | | | | Score = 351 bits (892), Expect = 4e−96 | |
| | | | | Identities = 181/247 (73%), Positives = 193/247 (77%) | |
| 22 | 28139 | 29098 | + | emb\|CAB56735.1\| (AL121600) ABC transporter protein, integra . . . 462 e−129 | ABC transporter (integral |
| | | | | pir\|\|G75293 probable manganese ABC transporter, permease pr . . . 208 1e−52 | membrane protein) |
| | | | | emb\|CAB56735.1\| (AL121600) ABC transporter protein, integral membrane subunit | Role in Mn or Fe transport |
| | | | | [Streptomyces coelicolor A3(2)] | |
| | | | | Length = 283 | |
| | | | | Score = 462 bits (1177), Expect = e−129 | |
| | | | | Identities = 241/272 (88%), Positives = 252/272 (92%) | |
| 23 | 29095 | 30285 | + | gi\|6002369\|emb\|CAB56734.1\| (AL121600) hypothetical protein . . . 484 e−136 | Hypothetical protein |
| | | | | gi\|13592175\|gb\|AAK31375.1\|AC084329_1 (AC084329) ppg3 [Leish . . . 61 2e−08 | |
| | | | | gi\|6002369\|emb\|CAB56734.1\| (AL121600) hypothetical protein SCF76.14c | |
| | | | | [Streptomyces coelicolor A3(2)] | |
| | | | | Length = 415 | |
| | | | | Score = 484 bits (1247), Expect = e−136 | |
| | | | | Identities = 245/395 (62%), Positives = 287/395 (72%), Gaps = 1/395 (0%) | |
| 24 | 30282 | 31244 | + | gi\|6002368\|emb\|CAB56733.1\| (AL121600) putative solute-bindi . . . 439 e−122 | ABC transporter protein |
| | | | | gi\|15807666\|ref\|NP_296243.1\|adhesin B [Deinococcus radiodu . . . 123 2e−27 | Translationally coupled to |
| | | | | gi\|6002368\|emb\|CAB56733.1\| (AL121600) putative solute-binding lipoprotein | orf 23 |
| | | | | [Streptomyces coelicolor A3(2)] | |
| | | | | Length = 329 | |
| | | | | Score = 439 bits (1128), Expect = e−122 | |
| | | | | Identities = 222/315 (70%), Positives = 253/315 (79%) | |
| 25 | 31332 | 32537 | + | emb\|CAB56732.1\| (AL121600) putative secreted protein [(Strep . . . 620 e−176 | Hypothetical Protein |
| | | | | gb\|AAA59875.1\| (M74027) mucin [Homo sapiens] 130 3e−29 | |
| | | | | emb\|CAB56732.1\| (AL121600) putative secreted protein [Streptomyces coelicolor A3(2)] | |
| | | | | Length = 402 | |
| | | | | Score = 620 bits (1581), Expect = e−176 | |
| | | | | Identities = 299/402 (74%), Positives = 341/402 (84%), Gaps = 1/402 (0%) | |
| 26a | 32816 | 33427 | − | gi\|8039818 HYPOTHETICAL 23.1 KDA PROTEIN MLCL581.27 159 5.3e−11 | Hypothetical protein |
| | | | | gi\|2829591 HYPOTHETICAL 23.0 KDA PROTEIN RV2637 143 4e−09 | |
| | | | | gi\|8039818\|sp\|Q49642\|YQ37_MYCLE | |
| | | | | HYPOTHETICAL 23.1 KDA PROTEIN MLCL581.27 | |
| | | | | Length = 214 | |
| | | | | Score = 159 (66.3 bits), Expect = 5.3e−11, P = 5.3e−11 | |
| | | | | Identities = 57/197 (28%), Positives = 166/197 (84%), Gaps = 14/197 (7%) | |
| 26b | 32686 | 32868 | + | gi\|15805506\|ref\|NP_294202.1\|penicillin-binding protein 1 [ . . . 33 0.72 | Hypothetical Protein |
| | | | | gi\|7248459\|gb\|AAF43497.1\|AF134579_1 (AF134579) arabinogalac . . . 32 0.95 | |
| | | | | gi\|15805506\|ref\|NP_294202.1\|penicillin-binding protein 1 [Deinococcus radiodurans] | |
| | | | | gi\|7473266\|pir\|\|B75514 penicillin-binding protein 1 - Deinococcus radiodurans (strain R1) | |
| | | | | gi\|6458167\|gb\|AAF10059.1\|AE001907_5 (AE001907) penicillin-binding protein | |
| | | | | 1 [Deinococcus radiodurans] | |
| | | | | Length = 873 | |
| | | | | Score = 32.7 bits (73), Expect = 0.72 | |
| | | | | Identities = 24/55 (43%), Positives = 28/55 (50%) | |
| 27 | 34195 | 35154 | + | pir\|\|T36741 probable ABC-type transport system ATP-binding . . . 291 6e−78 | Type I ABC transporter |
| | | | | gb\|AAD44229.1\|AF143772_35 (AF143772) DrrA [Mycobacterium av . . . 290 2e−77 | similar to daunorubicin |
| | | | | pir\|\|T36741 probable ABC-type transport system ATP-binding protein - | resistance gene, DrrA, in |
| | | | | Streptomyces coelicolor | Streptomyces antibioticus; |
| | | | | emb\|CAB50934.1\| (AL096849) putative ABC-transporter ATP-binding protein | has Walker A, B motifs. |
| | | | | [Streptomyces coelicolor A3(2)] | |
| | | | | Length = 332 | |
| | | | | Score = 291 bits (738), Expect = 6e−78 | |
| | | | | Identities = 168/303 (55%), Positives = 204/303 (66%), Gaps = 2/303 (0%) | |
| 28 | 35148 | 36017 | + | pir\|\|S32909 hypothetical protein 5 - Streptomyces antibioti . . . 120 2e−26 | ABC transporter (integral |
| | | | | pir\|\|T50567 probable ABC-type transport protein, transmembr . . . 115 6e−25 | membrane protein) similar |
| | | | | pir\|\|S32909 hypothetical protein 5 - Streptomyces antibioticus | to daunorubicin resistance |
| | | | | gb\|AAA26794.1\| (L06249) membrane protein [Streptomyces antibioticus] | gene, DrrB, in |
| | | | | Length = 273 | Streptomyces antibioticus; |
| | | | | Score = 120 bits (299), Expect = 2e−26 | has Walker A, B motifs. |
| | | | | Identities = 72/226 (31%), Positives = 113/226 (49%) | |
| 35 | 85270 | 85497 | + | pir\|\|T36310 probable small conserved hypothetical protein S . . . 111 9e−25 | Hypothetical Protein |
| | | | | gb\|AAG29779.1\|AF235050_2 (AF235050) CumB [Streptomyces rish . . . 101 1e−21 | |
| | | | | pir\|\|T36310 probable small conserved hypothetical protein SCE8.11c - | |
| | | | | Streptomyces coelicolor | |
| | | | | gb\|AAD18046.1\| (AF124138) Cda-orfX [Streptomyces coelicolor A3(2)] | |
| | | | | emb\|CAB38589.1\| (AL035654) putative small conserved hypothetical protein | |
| | | | | [Streptomyces coelicolor A3(2)] | |
| | | | | Length = 71 | |
| | | | | Score = 111 bits (276), Expect = 9e−25 | |
| | | | | Identities = 46/67 (68%), Positives = 56/67 (82%) | |
| 37 | 86434 | 87420 | + | pir\|\|T36307 hypothetical protein SCE8.08c - Streptomyces co . . . 175 7e−43 | Hypothetical Protein |
| | | | | gb\|AAA59875.1\| (M74027) mucin [Homo sapiens] 94 3e−18 | Translationally coupled to |
| | | | | pir\|\|T36307 hypothetical protein SCE8.08c - Streptomyces coelicolor | orf 38 |
| | | | | emb\|CAB38586.1\| (AL035654) hypothetical protein [Streptomyces coelicolor A3(2)] | |
| | | | | Length = 338 | |
| | | | | Score = 175 bits (439), Expect = 7e−43 | |
| | | | | Identities = 120/330 (36%), Positives = 164/330 (49%), Gaps = 13/330 (3%) | |
| 38 | 87417 | 88154 | + | pir\|\|E83323 hypothetical protein PA2579 [imported] - Pseudo . . . 102 3e−21 | Hypothetical Protein |
| | | | | pir\|\|G75588 probable tryptophan 2,3-dioxygenase - Deinococc . . . 87 2e−16 | Translationally coupled to |
| | | | | pir\|\|G75588 probable tryptophan 2,3-dioxygenase - Deinococcus radiodurans (strain R1) | orf37 |
| | | | | gb\|AAF12443.1\|AE001863_68 (AE001863) tryptophan 2,3-dioxygenase, | |
| | | | | putative [Deinococcus radiodurans] | |
| | | | | Length = 287 | |
| | | | | Score = 87.4 bits (213), Expect = 2e−16 | |
| | | | | Identities = 73/259 (28%), Positives = 107/259 (41%), Gaps = 37/259 (14%) | |
| 41 | 89910 | 90563 | + | gi\|7480757\|pir\|\|T36281 probable hydrolase - Streptomyces co . . . 114 2e−24 | |
| | | | | gi\|7480757\|pir\|\|T36281 probable hydrolase - Streptomyces coelicolor | |
| | | | | gi\|5123678\|emb\|CAB45367.1\|(AL079345) | |
| | | | | putative hydrolase [Streptomyces coelicolor A3(2)] | |
| | | | | Length = 215 Score = 114 bits (285), Expect = 2e−24 | |
| | | | | Identities = 72/170 (42%), Positives = 96/170 (56%), Gaps = 3/170 (1%) | |

Str refers to whether the gene is encoded on the DNA molecule (relative to SEQ ID NO: 1) from left to right (+) or from right to left on the complementary strand.
The BlastX box contains the two top BlastX scores for each ORF (top two lines) and details regarding the database protein entry and the alignment of the ORF to the database entry.

TABLE 7
BlastX Results for ORFs in SP6 Fragment

| ORF | start | stop | Str | BLASTX (accession numbers, entry title, P-value, E-value) | Polypeptide |
|---|---|---|---|---|---|
| 1 | 965 | 1 | − | pir\|\|T34645 hypothetical protein SC10H5.07 SC10H5.07 - Stre . . . 352 2e−96 | Hypothetical Protein |
| | | | | pir\|\|T36710 hypothetical protein SCH69.11c - Streptomyces c . . . 206 2e−52 | |
| | | | | pir\|\|T34645 hypothetical protein SC10H5.07 SC10H5.07 - Streptomyces coelicolor | |
| | | | | emb\|CAA20279.1\| (AL031232) hypothetical protein SC10H5.07 [Streptomyces coelicolor A3(2)] | |
| | | | | Length = 469 | |
| | | | | Score = 352 bits (904), Expect = 2e−96 | |
| | | | | Identities = 179/305 (58%), Positives = 216/305 (70%) | |
| 2 | 989 | 1948 | − | pir\|\|T35566 probable integral membrane protein - Streptomyc . . . 206 3e−52 | Hypothetical Protein |
| | | | | gb\|AAA53486.1\| (U03114) unknown [Streptomyces albus] 139 3e−32 | |
| | | | | pir\|\|T35566 probable integral membrane protein - Streptomyces coelicolor | |
| | | | | emb\|CAA20393.1\| (AL031317) putative integral membrane protein [Streptomyces | |
| | | | | coelicolor] | |
| | | | | Length = 315 | |
| | | | | Score = 206 bits (523), Expect = 3e−52 | |
| | | | | Identities = 114/311 (36%), Positives = 180/311 (57%), Gaps = 2/311 (0%) | |
| 3 | 2099 | 2392 | + | | Hypothetical Protein |
| 4 | 3277 | 2405 | − | emb\|CAB88937.1\| (AL353863) acyl-coA thioesterase [Streptomy . . . 535 e−151 | Acyl CoA |
| | | | | emb\|CAB87210.1\| (AL163641) acyl CoA thioesterase II [Strept . . . 293 1e−78 | thioesterase; |
| | | | | emb\|CAB88937.1\| (AL353863) acyl-coA thioesterase [Streptomyces coelicolor A3(2)] | enzyme involved |
| | | | | Length = 288 | in short chain |
| | | | | Score = 535 bits (1379), Expect = e−151 | fatty acid |
| | | | | Identities = 258/288 (89%), Positives = 273/288 (94%) | biosynthesis |
| 5 | 5885 | 3312 | − | emb\|CAB88936.1\| (AL353863) putative helicase [Streptomyces . . . 548 e−155 | DNA helicase |
| | | | | gb\|AAG45420.1\|AF309494_1 (AF309494) vegetative cell wall pr . . . 121 1e−26 | |
| | | | | emb\|CAB88936.1\| (AL353863) putative helicase [Streptomyces coelicolor A3(2)] | |
| | | | | Length = 854 | |
| | | | | Score = 548 bits (1413), Expect = e−155 | |
| | | | | Identities = 266/323 (82%), Positives = 291/323 (89%) | |
| 6 | 5963 | 6754 | + | emb\|CAB88935.1\| (AL353863) putative integral membrane prote . . . 491 e−138 | Hypothetical Protein |
| | | | | gb\|AAK31375.1\|AC084329_1 (AC084329) ppg3 [Leishmania major] 106 2e−22 | |
| | | | | emb\|CAB88935.1\| (AL353863) putative integral membrane protein [Streptomyces | |
| | | | | coelicolor A3(2)] | |
| | | | | Length = 264 | |
| | | | | Score = 491 bits (1265), Expect = e−138 | |
| | | | | Identities 235/264 (89%), Positives = 246/264 (93%), Gaps = 1/264 (0%) | |
| 7 | 6850 | 8403 | + | sp\|Q9FCB1\|DNLI_STRCO PROBABLE DNA LIGASE (POLYDEOXYRIBONUCL . . . 461 e−141 | DNA Ligase |
| | | | | ref\|NP_337667.1\| DNA ligase [Mycobacterium tuberculosis CDC . . . 294 4e−85 | |
| | | | | sp\|Q9FCB1\|DNLI_STRCO PROBABLE DNA LIGASE | |
| | | | | (POLYDEOXYRIBONUCLEOTIDE SYNTHASE [ATP]) | |
| | | | | emb\|CAC01484.1\| (AL391017) putative DNA ligase [Streptomyces coelicolor A3(2)] | |
| | | | | Length = 512 | |
| | | | | Score = 461 bits (1186), Expect(2) = e−141 | |
| | | | | Identities = 252/341 (73%), Positives = 267/341 (77%) | |
| 8 | 9860 | 8433 | − | emb\|CAB93757.1\| (AL357613) putative oxidoreductase. [Strept . . . 299 8e−81 | Oxidoreductase |
| | | | | pir\|\|T34726 probable dehydrogenase - Streptomyces coelicolo . . . 130 9e−30 | |
| | | | | emb\|CAB93757.1\| (AL357613) putative oxidoreductase. [Streptomyces coelicolor A3(2)] | |
| | | | | Length = 481 | |
| | | | | Score = 299 bits (766), Expect = 8e−81 | |
| | | | | Identities = 147/185 (79%), Positives = 165/185 (88%), Gaps = 1/185(0%) | |
| 9 | 10784 | 9921 | − | emb\|CAB57411.1\| (AL121746) hypothetical protein SCF73.06c [ . . . 311 3e−84 | Hypothetical Protein |
| | | | | gb\|AAK61383.1\| (AY035849) basic proline-rich protein [Sus s . . . 115 6e−25 | |
| | | | | emb\|CAB57411.1\| (AL121746) hypothetical protein SCF73.06c [Streptomyces coelicolor A3(2)] | |
| | | | | Length = 333 | |
| | | | | Score = 311 bits (798), Expect = 3e−84 | |
| | | | | Identities = 166/264 (62%), Positives = 182/264 (68%) | |

Str refers to whether the gene is encoded on the DNA molecule (relative to SEQ ID NO: 1) from left to right (+) or from right to left on the complementary strand.
The BlastX box contains the two top BlastX scores for each ORF (top two lines) and details regarding the database protein entry and the alignment of the ORF to the database entry.

TABLE 8
BlastX Results for ORFs in GTC Fragment

| ORF | start | stop | frame | DNA | Polypeptide |
|---|---|---|---|---|---|
| 2 | 2941 | 74 | −3 | >gi\|7435848\|pir\|\|S72176 thermolysin (EC 3.4.24.27) precursor - Bacillus caldolyticus (strain YP-T) | Thermostable |
| | | | | gi\|995782\|gb\|AAB18652.1\| (U25629) neutral proteinase [Bacillus caldolyticus] Length = 546 | neutral protein. |
| | | | | Score = 180 bits (457), Expect = 7e−44 | |
| | | | | Identities = 159/550 (28%), Positives = 251/550 (44%), Gaps = 20/550 (3%) | |
| 3 | 3078 | 4103 | 3 | >gi\|8977943\|emb\|CAB95810.1\| (AL359949) putative transcriptional regulator [Streptomyces | Positive |
| | | | | coelicolor A3(2)] Length = 343 | regulatory gene |
| | | | | Score = 89.0 bits (219), Expect = 7e−17 | for daptomycin |
| | | | | Identities = 93/335 (27%), Positives = 132/335 (38%), Gaps = 11/335 (3%) | synthesis |
| 4 | 5246 | 4131 | −2 | >gi\|6434729\|emb\|CAB61176.1\| (AL132973) putative DeoR-family transcriptional regulator | Negative |
| | | | | [Streptomyces coelicolor A3(2)] Length = 368 | regulatory gene |
| | | | | Score = 179 bits (454), Expect = 4e−44 | for daptomycin |
| | | | | Identities = 133/361 (36%), Positives = 171/361 (46%), Gaps = 7/361 (1%) | synthesis |
| 5 | 5536 | 6888 | 1 | >gi\|6434730\|emb\|CAB61177.1\| (AL132973) probable solute-binding lipoprotein. [Streptomyces | ABC Transporter |
| | | | | coelicolor A3(2)] Length = 443 | |
| | | | | Score = 226 bits (575), Expect = 7e−58 | |
| | | | | Identities = 129/358 (36%), Positives = 184/358 (51%), Gaps = 5/358 (1%) | |
| 6 | 7017 | 7814 | 3 | >gi\|6434731\|emb\|CAB61178.1\| (AL132973) putative binding protein dependent transport protein. | ABC Transporter |
| | | | | [Streptomyces coelicolor A3(2)] Length = 328 | |
| | | | | Score = 243 bits (619), Expect = 3e−63 | |
| | | | | Identities = 124/239 (51%), Positives = 163/239 (67%), Gaps = 5/239 (2%) | |
| 7 | 7943 | 8743 | 2 | >gi\|6434732\|emb\|CAB61179.1\| (AL132973) putative binding protein dependent transport protein. | ABC Transporter |
| | | | | [Streptomyces coelicolor A3(2)] Length = 287 | |
| | | | | Score = 265 bits (677), Expect = 5e−70 | |
| | | | | Identities = 131/252 (51%), Positives = 169/252 (66%) | |
| 8 | 8815 | 9795 | 1 | >gi\|6434733\|emb\|CAB61180.1\| (AL132973) putative 2-hydroxyacid-family dehydrogenase. | Dehydrogenase? |
| | | | | [Streptomyces coelicolor A3(2)] Length = 343 | |
| | | | | Score = 190 bits (482), Expect = 3e−47 | |
| | | | | Identities = 120/330 (36%), Positives = 166/330 (49%), Gaps = 1/330 (0%) | |
| 9 | 9843 | 11852 | 3 | | Hypothetical |
| | | | | | protein |
| 10 | 11860 | 12738 | 1 | | Hypothetical |
| | | | | | protein |
| 11 | 13799 | 12783 | −2 | | Hypothetical |
| | | | | | protein |
| 12 | 14051 | 14674 | 2 | | Hypothetical |
| | | | | | protein |
| 13 | 14671 | 15846 | 1 | >gi\|7480768\|pir\|\|T35943 probable hydrolytic protein - Streptomyces coelicolor | Hydrolase |
| | | | | gi\|4158202\|emb\|CAA22765.1\| (AL035206) putative hydrolytic protein [Streptomyces coelicolor | |
| | | | | A3(2)] | |
| | | | | Length = 464 | |
| | | | | Score = 145 bits (366), Expect = 8e−34 | |
| | | | | Identities = 78/198 (39%), Positives = 105/198 (52%), Gaps = 12/198 (6%) | |
| 14 | 15954 | 19265 | 3 | >gi\|8894813\|emb\|CAB96009.1\| (AL360055) hypothetical protein [Streptomyces coelicolor A3(2)] | SpoVK-like |
| | | | | Length = 833 | protein |
| | | | | Score = 369 bits (948), Expect = e−101 | |
| | | | | Identities = 271/831 (32%), Positives = 378/831 (44%), Gaps = 33/831 (3%) | |
| 15 | 19262 | 20530 | 2 | >gi\|7481390\|pir\|\|T42024 probable serine proteinase - Streptomyces coelicolor | Serine protease |
| | | | | gi\|1151075\|gb\|AAA85224.1\| (U33176) serine protease [Streptomyces coelicolor] | |
| | | | | Length = 390 | |
| | | | | Score = 68.2 bits (165), Expect = 2e−10 | |
| | | | | Identities = 63/209 (30%), Positives = 88/209 (41%), Gaps = 21/209 (10%) | |
| 16 | 23947 | 20585 | −2 | >gi\|3413388\|emb\|AL031231.1\|SC3C3 Streptomyces coelicolor cosmid 3C3 Length = 31382 | FtsK/SpoIIIE |
| | | | | Score = 421 bits (914), Expect(6) = 0.0 | homologue |
| | | | | Identities = 180/291 (61%), Positives = 222/291 (75%) | |

### EXAMPLE 15

### Heterologous Production of Daptomycin in Streptomyces lividans in the Absence of Actinorhodin

Both genetic and medium effects were modified to improve the expression of A21978C lipopeptides in an heterologous host. Various strains containing the dpt gene cluster BAC, along with control strains without the gene cluster, were grown in shake-flask fermentation and clarified broths analyzed for the presence of the A21978C lipopeptide series by HPLC. The dpt cluster on the BAC clone B1203A05 was introduced into S. lividans by protoplast generation using standard techniques (Keiser, T., et al., Practical Streptomycete genetics. John Innes Foundation, Norwich, 2000). Strains examined included both S. lividans TK23 and TK64 strains containing the dpt gene cluster and a genetically altered version of S. lividans TK23 with a partially deleted actinorhodin pathway. Other comparable and suitable act knockout strains are known to those in the art. TK64 differs from TK23 in possessing an rpsL (str-6) mutation conferring resistance to streptomycin, which has also been implicated in enhancement in the production of actinorhodin (Shima et al., J. Bacteriol, 178 (24), 7276-7284 (1996)). The actinorhodin family are colored polyketides produced in copious quantities by S. lividans under many fermentation conditions and which interfere with the detection and purification of other secondary metabolites from the fermentation.

To eliminate actinorhodin production from S. lividans, a cassette was constructed to delete part of the pathway. An 8 kb fragment containing the actinorhodin polyketide synthase pathway (Malpartida and Hopwood, Mol. Gen. Genet., 205, 66-73 (1986)) was cloned into a pUC19; 1.4 kb of DNA was removed from the center of this fragment, thus deleting the 3′ end of actIorfI and almost all of actIorfII. This fragment was then replaced by the resistance marker ermE (Bibb et al., Gene, 38 (1-3), 15-26 (1985)). This deletion cassette was then transferred to the temperature sensitive plasmid pGM160 and introduced into S. lividans TK23. These recombinant strains were then fermented for 40 hr before plating on to selective media, from this screening several colonies were isolated with the appropriate phenotype. The genotype of these strains was then confirmed by Southern blots. S. lividans strains of both TK64 and TK23 containing the BAC vector alone were also examined as control strains. See Table 9 for strain notation.

TABLE 9

| CBUK strain | | Presence | rpsL | |
|---|---|---|---|---|
| number | Lineage | of act | status | Transforming DNA |
| 136736 | TK64 | + | str-6 | BAC vector only |
| 136742 | TK64 | + | str-6 | B12: 03A05 dpt gene cluster |
| 137028 | TK23 | + | + | BAC vector only |
| 137027 | TK23 | + | + | B12: 03A05 dpt gene cluster |
| 137026 | TK23 | − | + | BAC vector only |
| | (521) | | | |
| 137024 | TK23 | − | + | B12: 03A05 dpt gene cluster |
| | (521) | | | |

+ in rpsL column indicates wild type status

Although a number of different media were initially explored, two different media were examined in more detail for their ability to support production of the A21978C lipopeptides in S. lividans. Both of these media also support good production of the A21978C lipopeptides in S. roseosporus. Medium A was a complex medium consisting of 1% glucose (BDH), 2% soluble starch (Sigma), 0.5% yeast extract (Difco), 0.5% casein (Sigma). 4.6% MOPS (Sigma), adjusted to pH 7 and autoclaved. Medium B was a defined medium consisting of 2% glycerol, 0.25% sucrose, 1.2% proline 1.5% MOPS, 0.056% K₂HPO₄, 0.05% NaCl, together with trace elements and vitamins, adjusted to pH 7 and filter sterilized.

Fermentations were initiated by inoculation of an enriched oatmeal slope containing 100 mg/L apramycin with approximately 0.25 ml material from a cryovial stored at −135° C. After 7-10 days incubation at 28° C., a mixed mycelial and spore suspension was generated by the addition of 4 ml 0.1% Tween 80 and 2 ml inoculated into 40 ml of seed medium containing 25 mg/L apramycin in a baffled flask to initiate the seed stage. Seed flasks were shaken at 240 rpm and 30° C. for 24-28 hours before a 5% transfer to production flasks containing 50 ml of medium A or B. Replicate flasks were sampled from day 2 until day 6 of the production fermentation period by aseptically removing approximately 1 ml broth, centrifuging for 10 min. at 10,000 rpm and analyzing the supernatant by HPLC. Analysis was performed at ambient temperature using a Waters Alliance 2690 HPLC system and a 996 PDA detector with a 4.6×50 mm Symmetry C8 3.5 μM column and a Phenomenex Security Guard C8 cartridge. The gradient initially holds at 90% water and 10% acetonitrile for 2.5 min., followed by a linear gradient over 6 minutes to 100% acetonitrile. The flow rate was 1.5 ml min⁻¹ and the gradient was buffered with 0.01% trifluoroacetic acid. Up to 50 microliters of the supernatant was injected to monitor for production of the native A21987C lipopeptides.

Confirmation of expected molecular weights was obtained by LC-MS analysis using a Finnigan SSQ710c system using electrospray ionization in positive ion mode, with a scan range of 200-2000 Daltons and 2 second scans. The LC method was run on a Waters Symmetry C8 column (2.1×50 mm 3.5 μm particle size). The method held at the initial conditions of 90% water, 10% acetonitrile and 0.01% formic acid for 0.5 minutes, followed by a linear gradient to 100% acetonitrile and 0.01% formic acid over 6 min. The method then held for 3.5 min. before re-equilibration. The method was run at ambient temperature.

The heterologous expression of the A21978C lipopeptide series in S. lividans TK64 (136742) in medium A was analyzed by HPLC. Production of three of the A21978C lipopeptides with characteristic UV/visible spectra was evident, with retention times of 5.61, 5.77 and 5.89 minutes (λmax 223.8, 261.5 and 364.5 nm) under the analytical conditions stated above. On LC-MS analysis, these three A21978C lipopeptides yielded molecular ions (M−H)⁺ at m/z of 1634.7, 1648.7 and 1662.7, which is in agreement with the masses reported for the major A21978C lipopeptide metabolites C₁, C₂ and C₃ respectively produced by Streptomyces roseosporus (Debono et al., J. Antibiotics, XL (6), 761-777 (1987)). A similar product profile was also obtained for heterologous expression of the dpt gene cluster in S. lividans TK23 (137027) under the same conditions. Similarly high production levels of actinorhodin were observed in this strain despite the absence of the rpsL mutation that is reported to enhance actinorhodin production. A21978C lipopeptides were not detected in fermentations of the TK64 control strain (136736) or the TK23 control strain (137028) with the BAC vector only integrated.

The amount of A21978C lipopeptides produced in crude broth by these strains could not be accurately quantitated due to co-chromotography with host peaks, including members of the CDA complex; however, a total maximum yield of the three main lipopeptides was estimated at approximately 20 mg/L. The A21978C lipopeptides were produced early on in the fermentation along with numerous other host metabolites.

The profile of production of the A21978C lipopeptides was also observed from fermentations of the S. lividans TK23 (137024) act knockout strain in medium A. Absence of an intact act pathway in this strain allowed application of the defined medium B, in which normally high levels of act are supported. Variations of the defined medium were evaluated and a 2 to 4 g/L level of K₂HPO₄ was found advantageous for both production of the A21978C lipopeptides and suppression of some of the host metabolites. HPLC analysis revealed a much cleaner HPLC profile obtained from crude broths of 137024 grown in a higher phosphate supplemented medium at 50 hrs, as compared to production of the A21978C lipopeptides in medium B without the phosphate supplementation. As the fermentation progressed and phosphate derepression occurred, the level and diversity of host metabolites increased, although never to the level previously observed in medium A. Although, early in the fermentation, the production of many host metabolites was suppressed, the production of the CDA series of lipopeptides was not. CDA can exist in both non-phosphorylated and phosphorylated forms. Under the chromatography conditions used, the non-phosphorylated forms of CDA co-chromatographed in the same region as the A21978C lipopeptides and complicated detection and quantification. Incorporation of phosphate into the fermentation medium biased production, at least initially, to the phosphorylated forms of CDA, which were well resolved from the three A21978C lipopeptides by HPLC. This effect on CDA production was also clearly evident from fermentation of the control strain of the S. lividans TK23 act knockout strain with an integrated BAC plasmid not containing the dpt gene cluster (137026) in high phosphate supplemented medium B.

1. An isolated nucleic acid molecule comprising a biosynthetic gene cluster, wherein the nucleic acid molecule comprises:
a) a nucleic acid sequence having the nucleic acid sequence SEQ ID NO: 1;
b) a nucleic acid sequence encoding the amino acid sequences SEQ ID NOs: 7, 8, 9, 11, 15 and 17;
c) a nucleic acid sequence with least 80% sequence identity to the nucleic acid sequence SEQ ID NO: 1;
d) a nucleic acid sequence with least 90% sequence identity to the nucleic acid sequence SEQ ID NO: 1;
e) a nucleic acid sequence with least 95% sequence identity to the nucleic acid sequence SEQ ID NO: 1;
f) a nucleic acid sequence encoding at least one adenylation domain, one condensation domain, one thiolation domain, and one epimerase domain;
g) a nucleic acid sequence encoding at least modules 1 to 13 of a daptomycin gene cluster;
h) a polynucleotide that hybridizes to a reference nucleic acid molecule comprising the nucleic acid sequence SEQ ID NO: 1 under stringent hybridization conditions having 6×SCC and 0% to 50% formamide at 42° C. to 68° C. for at least 10 hours;
i) a polynucleotide that hybridizes to a reference nucleic acid molecule comprising the nucleic acid sequence SEQ ID NO: 1 under stringent hybridization conditions having 6×SCC and 50% formamide at least 42° C. for at least 10 hours; or
j) a polynucleotide that hybridizes to a reference nucleic acid molecule comprising the nucleic acid sequence SEQ ID NO: 1 under stringent hybridization conditions having 6×SCC and 0% formamide at no more than 68° C. for at least 10 hours;
wherein the nucleic acid molecule encodes polypeptides capable of synthesizing a lipopeptide.

2. The nucleic acid molecule of claim 1, wherein the nucleic acid molecule comprises the nucleic acid sequence SEQ ID NO: 1.

3. The nucleic acid molecule of claim 1, wherein the nucleic acid molecule comprises a nucleic acid sequence encoding the amino acid sequences SEQ ID NOs: 7, 8, 9, 11, 15 and 17.

4. The nucleic acid molecule of claim 1, wherein the nucleic acid molecule comprises a nucleic acid sequence with least 80% sequence identity to the nucleic acid sequence SEQ ID NO: 1.

5. The nucleic acid molecule of claim 1, wherein the nucleic acid molecule comprises a nucleic acid sequence with least 90% sequence identity to the nucleic acid sequence SEQ ID NO: 1.

6. The nucleic acid molecule of claim 1, wherein the nucleic acid molecule comprises a nucleic acid sequence with least 95% sequence identity to the nucleic acid sequence SEQ ID NO: 1.

7. The nucleic acid molecule of claim 1, wherein the nucleic acid molecule hybridizes to a reference nucleic acid molecule comprising the nucleic acid sequence SEQ ID NO: 1 under stringent hybridization conditions having 6×SCC and 0% to 50% formamide at 42° C. to 68° C. for at least 10 hours.

8. The nucleic acid molecule of claim 1, wherein the nucleic acid molecule hybridizes to a reference nucleic acid molecule comprising the nucleic acid sequence SEQ ID NO: 1 under stringent hybridization conditions having 6×SCC and 50% formamide at least 42° C. for at least 10 hours.

9. The nucleic acid molecule of claim 1, wherein the nucleic acid molecule hybridizes to a reference nucleic acid molecule comprising the nucleic acid sequence SEQ ID NO: 1 under stringent hybridization conditions having 6×SCC and 0% formamide at no more than 68° C. for at least 10 hours.

10. The nucleic acid molecule of claim 1, wherein the nucleic acid molecule comprises a nucleic acid sequence encoding at least an adenylation domain, a condensation domain, a thiolation domain, and an epimerase domain.

11. The nucleic acid molecule of claim 1, wherein the nucleic acid molecule comprises a nucleic acid sequence encoding at least modules 1 to 13 of a daptomycin gene cluster.

12. The nucleic acid molecule of claim 1, wherein the nucleic acid molecule encodes polypeptides capable of synthesizing the lipopeptide A21978C1, A21978C2, or A21978C3.

13. A host cell comprising the nucleic acid molecule of claim 1.

14. A vector comprising the nucleic acid molecule of claim 1.

15. The vector of claim 12, wherein the vector comprises at least one expression control sequence controlling the transcription of the nucleic acid molecule.

16. The vector of claim 15, wherein the expression control sequence controls the expression of the nucleic acid molecule in a prokaryotic cell.

17. A host cell comprising the vector in claim 14.

18. A host cell comprising the vector in claim 15.

19. A host cell comprising the vector in claim 16.

20. A method of producing at least one polypeptide required for daptomycin synthesis comprising the step of culturing the host cell of claim 12, under conditions in which the polypeptide is produced, optionally comprising the step of isolating the polypeptide.

21. A method of producing at least one polypeptide required for daptomycin synthesis comprising the step of culturing the host cell of claim 17, under conditions in which the polypeptide is produced, optionally comprising the step of isolating the polypeptide.
